(19) United States
(12) Patent Application Publication
(10) Pub. No.: US 2026/0309671 A1
(43) Pub. Date: Oct. 8, 2026

(54) ELECTROCHEMICAL BIOSENSORS, SYSTEMS, AND METHODS OF USE

(71) Applicant: The Board of Trustees of the University of Illinois, Urbana, IL (US)

(72) Inventor: MATHEW THOPPIL MATHEW, ROCKFORD, IL (US)

(21) Appl. No.: 19/641,952

(22) Filed: Apr. 8, 2026

Related U.S. Application Data
(60) Provisional application No. 63/785,366, filed on Apr. 8, 2025.
(60) Provisional application No. 64/031,979, filed on Apr. 7, 2026.

(51) Int. Cl. G01N 27/327; G01N 33/543; G01N 33/551; G01N 33/57515; G01N 33/57525; G01N 33/68; G16B 40/20; G16B 40/30

(52) U.S. Cl. CPC G01N 27/3276; G01N 27/3278; G01N 33/54346; G01N 33/5438; G01N 33/551; G01N 33/57515; G01N 33/57525; G01N 33/6893; G01N 33/6896; G16B 40/20; G16B 40/30; G01N 2333/4737; G01N 2333/5412; G01N 2333/575; G01N 2333/96486; G01N 2410/00; G01N 2800/044; G01N 2800/105; G01N 2800/18; G01N 2800/26; G01N 2800/2871; G01N 2800/50

(57) ABSTRACT

Various examples are provided related to electrochemical biosensors, systems, and methods of use. A biomarker associated with a disease in a biological fluid sample can be detected by first contacting the biological fluid sample with a working electrode of an electrochemical biosensor. Next, one or more changes in an electronic property of the working electrode resulting from binding of the biomarker to the capture agent can be detected. Then, electrochemical data can be determined based at least in part on the one or more changes in the electronic property of the working electrode detected by the electrochemical biosensor. The electrochemical data can then be input into a machine-learning (ML) model trained to analyze the electrochemical data to determine a risk level associated with the disease. Finally, the risk level associated with the disease can be determined based at least in part on the ML model.

### CROSS REFERENCE TO RELATED APPLICATIONS

This application claims priority to, and the benefit of, U.S. provisional application entitled “Electrochemical Biosensors, Systems, and Methods of Use” having Ser. No. 64/031,979, filed Apr. 7, 2026, and U.S. provisional application entitled “Electrochemical Biosensors, Systems, and Methods of Use” having Ser. No. 63/785,366, filed Apr. 8, 2025, each of which is hereby incorporated by reference in its entirety.

### STATEMENT REGARDING FEDERALLY SPONSORED RESEARCH OR DEVELOPMENT

This invention was made with government support under Grant No. R01 DE031832 awarded by the National Institutes of Health. The government has certain rights in the invention.

### BACKGROUND

Biomarkers are biomolecules present in the body that can indicate normal or abnormal functioning, thus leading to the detection and monitoring of various diseases. For example, cancer-specific biomarkers can be produced by tumor-forming cells or other non-tumor cells in response to a tumor. In these examples, the presence of biomarkers can act as a sign of cancer and detecting them can serve as a prognosis for cancer. These biomarkers can include protein, RNA, DNA, and other metabolomics released into bodily fluids such as serum, blood, urine, and saliva. Proteins can be reliable biomarkers for various types of cancer as they directly reflect the effects and phenotype of cancerous cells. Further, proteins have many instrumentation options for their analysis.

Biosensors are analytical devices that detect a biorecognition event (e.g., antigen-antibody interaction, RNA-RNA or RNA-DNA hybridization, enzyme-substrate interaction, biomolecule-aptamer interaction, etc.) and convert it to a quantifiable output. There are various types of biosensors based at least in part on detection methods such as electrochemical, optical, mass, and calorimetric. For example, an electrochemical biosensor converts the biorecognition event (electrochemical reactions with the target molecule) into a quantifiable electrical output. However, current diagnostics are often costly and provide the results only at later stages of a disease. While biomarkers can aid early-stage diagnosis, conventional methods of detecting them can be limited in sensitivity and selectivity. In addition, current methods of detecting biomarkers are often time-consuming and dependent on well-trained professionals. Therefore, developing a diagnostic tool that can detect a disease at its early stages can benefit society by leading to improved patient outcomes.

### SUMMARY

Aspects of the present disclosure are related to electrochemical biosensors, systems, and methods of use.

The present disclosure presents examples of devices, systems, and methods, which will be described subsequently in greater detail, that can detect biomarkers in fluid samples.

The foregoing has thus outlined, rather broadly, features of the present disclosure in order that the present contribution may be better appreciated. There are additional features of present disclosure that will be described hereinafter and that will form the subject matter of the claims appended hereto.

In this respect, before explaining aspects of the present disclosure in detail, it is understood that the aspects of present disclosure are not limited in its application to the details of construction and to the arrangements of the components set forth in the following description or illustrated in the following drawings.

The aspects of present disclosure are capable of other embodiments and of being practiced and carried out in various ways. Also, it is to be understood that the phraseology and terminology employed herein are for the purpose of the description and should not be regarded as limiting.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments, are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a drawing of a network environment according to various embodiments of the present disclosure.

FIG. 2 is a flowchart illustrating one example of functionality implemented as a portion of a software application executed in a computing environment in the network environment of FIG. 1 according to various embodiments of the present disclosure.

FIGS. 3A-3E illustrate MMP9 protein calibration according to various embodiments of the present disclosure. FIG. 3A shows a bode plot and FIG. 3B shows a Nyquist plot. FIG. 3C shows EIS modelled change in impedance, while FIG. 3D shows percentage change in capacitance. FIG. 3E shows CV area.

FIGS. 4A-4E illustrate GSH protein calibration according to various embodiments of the present disclosure. FIG. 4A shows a bode plot and FIG. 4B shows a Nyquist plot. FIG. 4C shows EIS modelled change in impedance, while FIG. 4D shows percentage change in capacitance. FIG. 4E shows CV area.

FIGS. 5A and 5B illustrate EDS spectrum analysis elemental weight percentage comparison of different cases of antibody and protein of (FIG. 5C) MMP9 and (FIG. 5D) GSH in accordance with various embodiments of the present disclosure.

FIGS. 6A and 6B illustrate analysis of confocal images of antibody and protein interaction at 20× image for MMP9 (FIG. 6A) and GSH (FIG. 6B) using ImageJ software, in accordance with various embodiments of the present disclosure.

FIGS. 7A-7D illustrate (FIG. 7A) an ELISA analysis of MMP9, (FIG. 7B) an ELISA analysis of GSH, and SVM model confusion matrix of MMP9 (FIG. 7C) and GSH (FIG. 7D), in accordance with various embodiments of the present disclosure.

FIGS. 8A and 8B show biorecognition of an electrochemical biosensor to an electrical sensor for MMP9 (FIG. 8A) and GSH (FIG. 8B), in accordance with various embodiments of the present disclosure.

FIG. 9 shows an overview of data automation and ML prediction from electrochemical data measured by an electrochemical biosensor in accordance with various embodiments of the present disclosure.

FIGS. 10A and 10B are schematic representations of (FIG. 10A) an overview of the experimental setup and (FIG. 10B) the preparation of the biosensor in accordance with various embodiments of the present disclosure.

FIG. 11 is a schematic representation of the transduction system of the biosensor in accordance with various embodiments of the present disclosure. As shown in FIG. 11, 1) the analyte attracts to the receptor, 2) the receptor could be an enzyme, protein, nucleic acid, or antibody, electrical energy is generated by the chemical interaction between the analyte and the receptor, and 3) the signal is being transduced by the transducer (electrochemical, optical, or piezoelectric transducer) to the processor.

FIG. 12 shows the structure of an electrochemical biosensor in accordance with various embodiments of the present disclosure.

FIG. 13 is a schematic of a surface plasmon resonance-based biosensor in accordance with various embodiments of the present disclosure. The gold film can be coated with biorecognition molecules which are antibodies. The biorecognition molecules can interact with the biomarkers and the interaction between the antibodies and antigens result in changes in the refractive index that leads to a shift in the angle of SPR.

FIG. 14 is an illustration of the principle of the colorimetric based biosensor in accordance with various embodiments of the present disclosure.

FIG. 15 is an illustration of a chemiluminescence-based biosensor in accordance with various embodiments of the present disclosure. The interaction between recognition elements such as antibody and the biomarkers can result in light emission.

FIG. 16 is a schematic of the structure and principle of the piezoelectric biosensor in accordance with various embodiments of the present disclosure.

FIG. 17 is a schematic diagram that explains the principle of EIS in accordance with various embodiments of the present disclosure. An AC voltage of 10 mV is applied across the working electrode of the biosensor (gold) and the current variation is measured.

FIGS. 18A-18D show EIS analysis in accordance with various embodiments of the present disclosure. FIG. 18A shows the electrochemical double layer with protein molecules, while FIG. 18B shows the typical EIS data as Nyquist plot and Bode plot. FIG. 18C shows the equivalent circuit and FIG. 18D shows the Randles circuit with EDL.

FIG. 19 shows an experimental protocol for the electrochemical experiments in accordance with various embodiments of the present disclosure.

FIGS. 20A-20D show the electrochemical experimental results obtained for the optimization of the CA 19-9 antibody using a gold electrode, including (FIG. 20A) a bode plot, (FIG. 20B) a Nyquist plot, (FIG. 20C) a % change in capacitance, and (FIG. 20D) a change in impedance in accordance with various embodiments of the present disclosure.

FIG. 21 shows the concept of the affinity between the antibody and the antigen in accordance with various embodiments of the present disclosure.

FIG. 22 shows a bode plot for the control which is urine and antibody, 10 pg/mL, 50 pg/mL ECM1 in accordance with various embodiments of the present disclosure. The bode plot shows that the order of the impedance is as follows 10 pg/mL>control>50 pg/mL. The bode plot shows that the data are not in order for increasing or decreasing.

FIG. 23 shows a bode plot for the control which is urine and antibody, 10 pg/mL, 50 pg/mL, 100 pg/mL ECM1 in accordance with various embodiments of the present disclosure. The bode plot shows that the order of the impedance is as follows 50 pg/mL>10 pg/mL>control>100 pg/mL. The data are not in order and not consistent.

FIG. 24 shows change in impedance for 10, 50, 100 pg/mL ECM1 in different frequencies which are 24.9, 7.8, 0.0125 Hz in accordance with various embodiments of the present disclosure. The graph shows that 0.0125 Hz has a maximum change in impedance in all ECM1 concentrations. Thus, 0.0125 Hz is the optimum frequency for ECM1 detection comparing with 24.9 Hz and 7.8 Hz. ΔZ increased from 10 pg/mL to 100 pg/mL ECM1.

FIG. 25 shows the change in capacitance for 10, 50, 100 pg/mL ECM1 in accordance with various embodiments of the present disclosure. The change in capacitance increases for 10 and 50 pg/mL ECM1, but it decreases for 100 pg/mL ECM1. For 10 pg/mL, the change in capacitance is around 1%. For 50 pg/mL, the change in capacitance is around 6%. For 100 pg/mL, the change in capacitance is between 3 and 4%.

FIG. 26 shows a bode plot for the control which is urine and antibody, 10 pg/mL, 50 pg/mL, 200 pg/mL of ECM1 in accordance with various embodiments of the present disclosure. The bode plot shows that the order of the impedance is as follows control>200 pg/mL>50 pg/mL>100 pg/mL ECM1.

FIG. 27 shows the change in impedance for 10, 50, 200 pg/mL ECM1 in different frequencies which are 24.9, 7.8, 0.0125 Hz in accordance with various embodiments of the present disclosure. The graph shows that 0.0125 Hz has a maximum change in impedance in all ECM1 concentrations. Thus, 0.0125 Hz is the optimum frequency for ECM1 detection comparing with 24.9 Hz and 7.8 Hz. ΔZ decreased from 10 pg/mL to 100 pg/mL ECM1.

FIG. 28 shows the change in capacitance for 10, 50, 200 pg/mL ECM1 in accordance with various embodiments of the present disclosure. The change in capacitance decreased from 10 to 200 pg/mL ECM1. For 10 pg/mL, the change in capacitance is around 22%. For 50 pg/mL, the change in capacitance is around 15%. For 200 pg/mL, the change in capacitance is near 8%.

FIG. 29 shows a bode plot for the antibody concentrations, which are 15 μg/mL, 10 μg/mL, 7 μg/mL, 5 μg/mL, 4 μg/mL, 1 μg/mL in accordance with various embodiments of the present disclosure. The bode plot shows that the order of the impedance is as follows 10 μg/mL>15 μg/mL>7 μg/mL>5 g/mL>4 μg/mL>1 μg/mL hECM1 antibody.

FIG. 30 shows the % change in impedance at 0.0125 Hz in accordance with various embodiments of the present disclosure. The order of the % change in impedance is as follows 10 μg/mL>15 μg/mL>7 μg/mL>5 μg/mL>4 μg/mL>2 μg/mL>1 μg/mL>0.5 g/mL hECM1 antibody in accordance with various embodiments of the present disclosure. 10 μg/mL is the optimal concentration of the antibody on the sensor. Values represented as mean±standard deviation (SD) using student t-test, n=3, *p<0.05.

FIG. 31 shows the % change of the capacitance at 0.0125 Hz in accordance with various embodiments of the present disclosure. The graphs show that the percentage changes in capacitance increases as a function of the increase in the concentration from 0.5 μg/mL of anti-ECM antibody to 10 μg/mL. Then, when the concentration reached to 15 μg/mL, the % change in capacitance decreases. Hence, the optimal concentration of the antibody on the sensor is considered as 10 μg/mL (see the circle). Data expressed as ± standard deviation (SD) using student t-test, n=3, *p<0.05, ** p<0.01.

FIG. 32 shows a bode plot for the 1 pg/mL ECM1, 5 pg/mL ECM1, 10 pg/mL ECM1, 20 pg/mL ECM1, 50 pg/mL ECM1, 100 pg/mL ECM1 in accordance with various embodiments of the present disclosure. The bode plot shows that the order of the impedance is as follows 5 pg/mL>20 pg/mL>10 pg/mL>50 pg/mL>1 pg/mL>100 pg/mL ECM1.

FIG. 33 shows a change in impedance for 1, 5, 10, 20, 50, and 100 pg/mL at 0.0125 Hz in accordance with various embodiments of the present disclosure. The graph shows that 100 pg/mL has a maximum change in impedance in all ECM1 concentrations. However, 20 pg/mL has a minimum change in impedance in all ECM1 concentrations. The graph indicates that the impedance of the ECM1 concentrations is not consistent.

FIG. 34 shows the change in capacitance for 1, 5, 10, 20, 50, and 100 pg/mL at 0.0125 Hz in accordance with various embodiments of the present disclosure. The graph shows that 100 pg/mL has a maximum change in capacitance in all ECM1 concentrations. The graph shows that the impedance of the ECM1 concentrations are not consistent because the % change increases and decreases as a function of ECM1 concentration.

FIG. 35 shows open circuit potential where each sensor has different OCP, in accordance with various embodiments of the present disclosure.

FIG. 36 shows a bode plot for the control which is urine and antibody, 10 pg/ml of ECM1, 100 pg/mL of ECM1, and 500 pg/mL of ECM1 in accordance with various embodiments of the present disclosure. The bode plot shows that the order of the impedance is as follows 500 pg/mL>100 pg/mL>10 pg/mL>control. The impedance is in the order of increasing value.

FIG. 37 shows the change in impedance for 10, 100, and 500 pg/mL at 0.0125 Hz in accordance with various embodiments of the present disclosure. The graph shows that ΔZ sharply increases from 10 pg/mL to 500 pg/mL ECM1. ΔZ for 10 pg/mL is around 100000 ohms, ΔZ for 100 pg/mL is around 130000 ohms, and ΔZ for 500 pg/mL is around 180000 ohms. Values represented as mean±standard deviation (SD) using student t-test, n=3, *p<0.05, ** p<0.01.

FIG. 38 shows the % change of the capacitance at 0.0125 Hz in accordance with various embodiments of the present disclosure. The graphs show that the percentage changes in capacitance increases as a function of the increase in the concentration from 10 pg/mL to 500 pg/mL. Values represented as mean±standard deviation (SD) using student t-test, n=3, *p<0.05.

FIGS. 39A-39E show the electrochemical experimental results obtained for the calibration of the CA 19-9 antigen or the detection of CA 19-9 protein using a gold electrode with an optimized CA 19-9 antibody obtained from previous experiments, including (FIG. 39A) a bode plot, (FIG. 39B) a Nyquist plot, (FIG. 39C) % change in capacitance, (FIG. 39D) change in impedance, and (FIG. 39E) cyclic voltammetry (CV) area graphs in accordance with various embodiments of the present disclosure.

FIG. 40 shows the fluorescent intensity image of ECM1 by using image J software in accordance with various embodiments of the present disclosure. There is a significant increase in the fluorescent intensity in the case of 5 ng/ml compared to 1 pg/mL concentration of ECM1.

FIG. 41 shows a bode plot for the control which is urine and antibody, 1 pg/ml of ECM1, 10 pg/mL of ECM1, 500 pg/mL of ECM1, 1 ng/ml of ECM1, 500 ng/ML of ECM1, 1 pg/mL of ECM1 in accordance with various embodiments of the present disclosure. The bode plot shows that the order of the impedance is as follows control<1 pg/mL<10 pg/mL<500 pg/mL<1 ng/ml<500 ng/ml<1 μg/mL ECM1.

FIG. 42 shows the change in impedance for 1 pg/mL, 10 pg/mL, 100 pg/mL, 500 pg/mL, 1 ng/ml, 500 ng/mL, 1 pg/mL at 0.0125 Hz in accordance with various embodiments of the present disclosure. The graph shows that ΔZ increases from 10 pg/mL to 1 μg/mL ECM1. Values represented as mean±standard deviation (SD) using student t-test, n=3, *p<0.05.

FIG. 43 shows the % change of the capacitance at 0.0125 Hz in accordance with various embodiments of the present disclosure. The graphs show a clear increase in percentage changes in capacitance and in impedance from 1 pg/mL to 1 μg/mL. There is a linear increase in impedance from 1 pg/mL to 1 μg/mL of ECM1 protein in PBS. Values represented as mean±standard deviation (SD) using student t-test, n=3, *p<0.05, ** p<0.01.

FIG. 44 shows a bode plot for the control which is urine and antibody, 1 pg/ml of ECM1, 10 pg/mL of ECM1, 100 pg/mL, 500 pg/mL of ECM1, 1 ng/ml of ECM1, 500 ng/ml of ECM1, 1 μg/mL of ECM1 in accordance with various embodiments of the present disclosure. The bode plot shows that the order of the impedance is as follows control<1 pg/mL<10 pg/mL<100 pg/mL<1 μg/mL<500 ng/ml<500 pg/mL<1 ng/mL ECM1.

FIG. 45 shows the change in impedance for 1 pg/mL, 10 pg/mL, 100 pg/mL, 500 pg/mL, 1 ng/ml, 500 ng/mL, 1 μg/mL at 0.0125 Hz in accordance with various embodiments of the present disclosure. The graph shows that ΔZ sharply increases from 1 pg/mL to 1 ng/ml ECM1, the ΔZ decreases from 500 ng/mL to 1 pg/mL ECM1. Values represented as mean±standard deviation (SD) using student t-test, n=3, *p<0.05, ** p<0.01.

FIG. 46 shows the % change of the capacitance at 0.0125 Hz in accordance with various embodiments of the present disclosure. The graphs show a clear increase in percentage changes in capacitance from 1 pg/mL to 1 ng/ml. There is a linear increase in capacitance lower than 500 ng/ml of ECM1 protein in the synthetic urine. Values represented as mean±standard deviation (SD) using student t-test, n=3, *p<0.05, ** p<0.01.

FIG. 47 shows ELISA serial dilution of ECM1 protein in accordance with various embodiments of the present disclosure. The graph shows that the protein is steady from 0.12 ng to 1.95, then the protein increases at 4 ng/ml and above. Values represented as mean±standard deviation (SD) using student t-test, n=3, *p<0.05, ** p<0.01.

FIG. 48 shows a standard curve of the ECM1 from the urine in accordance with various embodiments of the present disclosure.

FIG. 49 shows a standard curve of the ECM1 from the urine in accordance with various embodiments of the present disclosure. The estimated concentration from the blind test 1 was 7.4 pg/mL ECM1.

FIG. 50 shows a standard curve of the ECM1 from the urine in accordance with various embodiments of the present disclosure. The estimated concentration from the blind test 2 was 48.6 pg/mL ECM1.

FIG. 51 shows a standard curve of the ECM1 from the urine in accordance with various embodiments of the present disclosure. The estimated concentration from the blind test 3 was 110 pg/mL ECM1.

FIG. 52 shows the overall mechanism of the electrochemical biosensor in accordance with various embodiments of the present disclosure.

FIG. 53 shows a breast cancer detection kit in accordance with various embodiments of the present disclosure.

FIG. 54 shows protein calibration for artificial serum and artificial saliva detected by an electrochemical biosensor configured to detect adiponectin in accordance with various embodiments of the present disclosure.

FIG. 55 shows SVM models for artificial serum, artificial saliva, and SVM with RBF kernel in artificial saliva in accordance with various embodiments of the present disclosure.

FIG. 56 shows a structure of an electrochemical biosensor configured to detect CA 19-9 in accordance with various embodiments of the present disclosure.

FIGS. 57A-57C show detection of CA 19-9 protein using (FIG. 57A) electrochemical analysis, (FIG. 57B) ELISA, n=3, p<0.0001, and (FIG. 57C) machine learning in predicting risk levels of CA 19-9 protein in accordance with various embodiments of the present disclosure.

FIGS. 58A-58D show an overview of development of an electrochemical biosensor for detection of risk level of pancreatic cancer in accordance with various embodiments of the present disclosure. FIG. 58A shows various biomarkers associated with stages of pancreatic cancer and the improvements over current methods of detection. FIG. 58B shows the fabrication of the nanoengineered biosensor. FIG. 58C shows the calibration and validation of protein detection using the biosensor. FIG. 58D shows machine learning model development in predicting risk levels.

FIGS. 59A-59D show the electrochemical results obtained for the optimization of the CA 19-9 antibody using a graphene oxide coated gold electrode, including (FIG. 59A) a bode plot, (FIG. 59B) a Nyquist plot, (FIG. 59C) % change in capacitance, and (FIG. 59D) change in impedance in accordance with various embodiments of the present disclosure.

FIGS. 60A-60E show the electrochemical experimental results obtained for the calibration of the CA 19-9 antigen or the detection of CA 19-9 protein using a graphene-coated gold electrode with an optimized CA 19-9 antibody obtained from the previous experiments, including (FIG. 60A) a bode plot, (FIG. 60B) a Nyquist plot, (FIG. 60C) % change in capacitance, (FIG. 60D) change in impedance, and (FIG. 60E) cyclic voltammetry (CV) area graphs in accordance with various embodiments of the present disclosure.

FIGS. 61A-61D show the electrochemical results obtained for the optimization of the CA 19-9 antibody using a graphene oxide electrode, including (FIG. 61A) a bode plot, (FIG. 61B) a Nyquist plot, (FIG. 61C) % change in capacitance, and (FIG. 61D) change in impedance in accordance with various embodiments of the present disclosure.

FIGS. 62A-62E show the electrochemical experimental results obtained for the calibration of the CA 19-9 antigen for the detection of CA 19-9 protein using a graphene oxide electrode with an optimized CA 19-9 antibody obtained from the previous experiments, including (FIG. 62A) a bode plot, (FIG. 62B) a Nyquist plot, (FIG. 62C) % change in capacitance, (FIG. 62D) change in impedance, and (FIG. 62E) CV area graphs in accordance with various embodiments of the present disclosure.

FIG. 63 shows in vitro biological analysis—ELISA test—to compare the sensitivity of the developed electrochemical biosensor with the conventional biological assay in accordance with various embodiments of the present disclosure.

FIGS. 64A and 64B show electrochemical results compared with the automation model results in accordance with various embodiments of the present disclosure. The scatter plot (FIG. 64A) and the confusion matrix (FIG. 64B) were drawn from the results of the automation (SVM) model and 95.45% accuracy was achieved.

FIG. 65 shows a mechanism in accordance with various embodiments of the present disclosure.

FIG. 66 shows protein calibration in accordance with various embodiments of the present disclosure.

FIG. 67 shows an automation model in accordance with various embodiments of the present disclosure.

FIG. 68 shows a machine learning model in accordance with various embodiments of the present disclosure.

FIGS. 69A and 69B show antibody saturation in accordance with various embodiments of the present disclosure. FIG. 69A shows a change in impedance for IL6, while FIG. 69B shows a change in impedance for CRP. The change in impedance is maximized at maximum efficacy. Minimal change in capacitance affirms trends in data. 1-way-ANOVA; * p<0.05, ** p<0.01.

FIGS. 70A-70H show protein calibration curves in accordance with various embodiments of the present disclosure. FIG. 70A shows a change in impedance for IL6, while FIG. 70B shows a % change in capacitance for IL6. FIG. 70C shows a Nyquist plot for IL6, while FIG. 70D shows a Bode plot for IL6. FIG. 70E shows a change in impedance for CRP, while FIG. 70F shows a % change in capacitance for CRP. FIG. 70G shows a Nyquist plot for CRP, while FIG. 70H shows a Bode plot for CRP. 1-way-ANOVA; * p<0.05, ** p<0.01, *** p<0.001.

FIGS. 71A and 71B show confusion matrices in accordance with various embodiments of the present disclosure. FIG. 71A shows the trained SVM model yielded a 70.77% accuracy for IL6 and FIG. 71B shows the model yielded a 72.41% accuracy for CRP.

FIGS. 72A-72E show results of the simulated infection model validation and blind test validation in accordance with various embodiments of the present disclosure. FIG. 72A shows the fluorescent intensity measurement using ImageJ software, while FIG. 72B shows ELISA analysis of HFLS supernatant concentration. FIG. 72C shows biosensor validation results. The biosensor successfully validated an initial test and identified the supernatant as high-risk for PJI. FIG. 72D and FIG. 72E show the biosensor measured change in impedance values that correlate with the actual concentrations for both IL6 (FIG. 72D) and CRP (FIG. 72E).

FIGS. 73A and 73B show antibody saturation in accordance with various embodiments of the present disclosure. FIG. 73A shows change in impedance, while FIG. 73B shows % change in capacitance. The optimal antibody was found to be 1 μg/mL.

FIGS. 74A-74D show protein calibrations in serum in accordance with various embodiments of the present disclosure. FIG. 74A shows change in impedance, FIG. 74B shows % change in capacitance, FIG. 74C shows change in resistance, and FIG. 74D shows CV area. Ordinary one-way Anova *P<0.05.

FIGS. 75A-75D show protein calibrations in saliva in accordance with various embodiments of the present disclosure. FIG. 75A shows change in impedance, FIG. 75B shows % change in capacitance, FIG. 75C shows change in resistance, and FIG. 75D shows CV area. Ordinary one-way Anova P<0.05 *; P<0.01 **; P<0.001 ***; P<0.0001 ****.

FIG. 76 shows concentration of HFLS treated with 100 μg/mL of LPS and HFLS treated with 500 ng/ml of LTA with ELISA in accordance with various embodiments of the present disclosure.

FIGS. 77A-77C show biosensor validation results in accordance with various embodiments of the present disclosure. FIG. 77A shows change in impedance, FIG. 77B shows % change in capacitance, FIG. 77C shows change in resistance for HFLS treated with 100 μg/mL of LPS and HFLS treated with 500 ng/ml of LTA.

FIGS. 78A and 78B show SVM ML model validation in accordance with various embodiments of the present disclosure. FIG. 78A shows SVM model of data from different COMP concentrations in artificial serum with the accuracy of 68.18%. FIG. 78B shows SVM model of data from different COMP concentrations in artificial saliva with an accuracy of 72.73%.

### DETAILED DESCRIPTION

Disclosed herein are various examples related to electrochemical biosensors, systems, and methods of use. Reference will now be made in detail to the description of the embodiments as illustrated in the drawings, wherein like reference numbers indicate like parts throughout the several views.

The present disclosure relates to electrochemical biosensor systems configured for detecting biomarkers in biological fluids. An electrochemical biosensor for detecting a biomarker in a biological fluid can include a working electrode, a reference electrode, and a counter electrode arranged to measure changes in electronic properties when a target biomarker binds to a capture agent disposed on the working electrode. The electrochemical biosensor can be configured to analyze various biological fluids to identify the presence and concentration of biomarkers associated with various disease states.

A method of detecting a biomarker associated with a disease in a biological fluid sample can involve contacting the biological fluid sample with the working electrode of the electrochemical biosensor, detecting changes in electronic properties resulting from biomarker binding, and processing the resulting electrochemical data to determine disease-related information. The method can combine electrochemical detection techniques with computational analysis to provide risk assessment information based on biomarker concentrations detected in the biological fluid sample.

A disease risk prediction system can include an electrochemical biosensor, a computing device comprising a processor and a memory, and machine-readable instructions stored in the memory. The computing device can be configured to receive electrochemical data from the electrochemical biosensor and process the electrochemical data using a machine-learning model trained to analyze the electrochemical data. The machine-learning model can be configured to determine a risk level associated with a disease based on the electrochemical data. The disease risk prediction system can provide a non-invasive approach for early detection and monitoring of various disease states by analyzing biomarkers present in biological fluids such as saliva, urine, serum, or blood.

The electrochemical biosensor systems described herein can utilize antibody-antigen interactions to selectively capture target biomarkers from biological fluid samples. When a target biomarker binds to a capture agent on the working electrode surface, the binding event can cause measurable changes in electronic properties of the working electrode. These changes can be detected using electrochemical techniques such as electrochemical impedance spectroscopy or cyclic voltammetry. The resulting electrochemical data can then be processed by the computing device and analyzed using the machine-learning model to determine a risk level associated with a particular disease.

When an electrode comprising nanostructures on its surface is exposed to liquid containing biomolecules, a potential difference can be created at the electrode/electrolyte interface due to unequal distribution of charges. As a result of biomolecular binding events at the nanostructured electrode surface, redistribution of charges in the electrode and ions in the electrolyte can result in formation of a space-charge region within the nanostructures and at an electrical double layer at the electrode/electrolyte interface. Biomarker binding can be evaluated and quantified by measuring changes in electrode impedance and/or capacitance at selected frequencies. In some embodiments, changes to the space-charge capacitance and overall impedance at the electrode/electrolyte interface can be measured using both Mott-Schottky technique and a modified electrochemical impedance spectroscopy (EIS) technique which are described in detail herein. A correlation in output signal response with concentration can be determined between (and using) both detection techniques, which provide a combinatorial approach for the accurate and sensitive detection of protein biomarkers.

The electrochemical biosensor can be configured as a three-electrode system comprising a working electrode, a reference electrode, and a counter electrode. The three-electrode system configuration can provide a stable electrochemical measurement environment for detecting biomarkers in biological fluid samples.

The electrodes can be exposed to a liquid sample suspected to contain one or more target biomarkers. A working electrode as described herein can be referred to interchangeably as a sensing electrode, a sensing working electrode, detection electrode, or the like. The working electrode further comprises one or more capture reagents as described herein conjugated to its surface. The reference electrode and counter electrode need not comprise capture reagents on their surfaces. The counter electrode and reference electrode may be electrochemically inert/stable, and collectively form an electrochemical cell with the working electrode when the electrodes come into contact with the fluid sample.

The fluid sample can comprise any suitable fluid including liquids, gels, colloids, gases and/or combination thereof. Examples of fluids include body fluids, such as sweat, blood, tears, serum, saliva, urine, etc.; and non-body fluids such as vapors (from fruits, milk, and other foods), aqueous and non-aqueous solutions, etc. The biomarker can correspond to the various biomolecules being tested.

The working electrode can comprise a gold electrode. In some cases, the working electrode has a diameter of approximately 4 mm. The gold working electrode can serve as the sensing surface where capture agents are disposed for selective binding of target biomarkers. The gold material of the working electrode can provide a conductive surface suitable for electrochemical measurements and for conjugation of capture agents.

The reference electrode can comprise a platinum electrode. The platinum reference electrode can provide a stable reference potential against which measurements at the working electrode are made. The reference electrode can maintain a constant potential during electrochemical measurements, allowing for accurate detection of changes in electronic properties at the working electrode surface.

The counter electrode can comprise a carbon electrode. The carbon counter electrode can complete the electrical circuit of the three-electrode system. The counter electrode can allow current to flow through the electrochemical cell during measurements without affecting the reference electrode potential.

The three-electrode system can be arranged on a substrate to form a screen-printed electrode configuration. The arrangement of the working electrode, the reference electrode, and the counter electrode on the substrate can provide a compact and reproducible sensor platform. The screen-printed electrode configuration can allow for consistent electrode spacing and geometry across multiple biosensor units. In various aspects, the substrate can be rigid or flexible. The substrate can include silicon, glass, printed circuit boards, polyurethane, polycarbonate, polyamide, polyimide, and the like.

The electrochemical biosensor includes a working electrode configured for detecting biomarkers in biological fluid samples. As described above, the working electrode can comprise a gold electrode that serves as the sensing surface for biomarker detection. The working electrode can be configured in various embodiments to provide different surface characteristics for capture agent immobilization and biomarker binding.

In some cases, the working electrode comprises a gold screen-printed electrode without a graphene oxide coating. In such embodiments, the gold surface of the working electrode can be used directly for antibody immobilization. The gold surface can provide suitable binding sites for conjugation of capture agents through various chemical linkage mechanisms.

In some cases, the working electrode comprises a gold electrode coated with graphene oxide nanoparticles. The graphene oxide nanoparticles can be disposed on the gold electrode surface to provide an enhanced surface area for capture agent conjugation. The working electrode comprising a surface coated with graphene oxide nanoparticles can provide increased binding capacity for capture agents compared to an uncoated gold surface. Graphene oxide is a nanomaterial with a two-dimensional structure that originates from graphene, which includes a single sheet of carbon atoms organized in a hexagonal lattice. The unique suitability of graphene oxide for biosensors arises from its remarkable surface area, mechanical strength, and outstanding electrical conductivity. These characteristics allow for its interaction with biomolecules and the facilitation of detection systems that are exceedingly sensitive. The extensive surface area of the material offers many binding sites for the selective biomarkers (the selected diseases). The enhanced binding capacity of the biosensor enables the detection of extremely low quantities of these biomarkers, hence significantly advancing the limits of sensitivity beyond what was previously achievable.

The graphene oxide nanoparticles can be applied to the gold electrode surface using a graphene oxide nanocolloidal solution. In some cases, the graphene oxide nanocolloidal solution has a concentration of approximately 2 mg/mL dispersed in water. The graphene oxide nanocolloidal solution can be deposited onto the gold electrode surface and allowed to dry to form a coating of graphene oxide nanoparticles.

The graphene oxide nanoparticles can be crosslinked to the gold electrode surface by exposure to ultraviolet radiation. In some cases, the ultraviolet crosslinking process is performed overnight to achieve stable attachment of the graphene oxide nanoparticles to the gold electrode surface. The ultraviolet crosslinking process can create covalent bonds between the graphene oxide nanoparticles and the gold electrode surface, providing a stable coating that remains attached during subsequent processing steps and during contact with biological fluid samples.

In some cases, the working electrode comprises a graphene oxide screen-printed electrode as an alternative to gold electrodes. The graphene oxide screen-printed electrode can provide a working electrode surface composed primarily of graphene oxide material rather than a gold electrode coated with graphene oxide nanoparticles. The graphene oxide screen-printed electrode can offer different electrochemical characteristics compared to gold-based working electrodes.

When contacting the biological fluid sample with a working electrode of an electrochemical biosensor, the working electrode surface interacts with the biological fluid sample to allow target biomarkers to bind to capture agents disposed on the working electrode. The working electrode comprising a surface coated with graphene oxide nanoparticles and a capture agent conjugated to the graphene oxide nanoparticles can provide selective binding of target biomarkers from the biological fluid sample. The capture agent conjugated to the graphene oxide nanoparticles can selectively bind to target biomarkers present in the biological fluid sample, resulting in detectable changes in electronic properties of the working electrode.

The electrochemical biosensor includes a capture agent conjugated to the graphene oxide nanoparticles. The capture agent is configured to selectively bind to a target biomarker in the biological fluid. The capture agent can be disposed on a surface of the working electrode and configured to selectively bind to a target biomarker associated with a disease. The selective binding of the capture agent to the target biomarker can enable detection of specific biomarkers present in biological fluid samples.

In some cases, the capture agent comprises an antibody or an antibody fragment. Antibodies can provide high specificity for target biomarkers due to the complementary binding sites between the antibody and the target biomarker antigen. Antibody fragments, such as Fab fragments or single-chain variable fragments, can also be used as capture agents and can provide similar binding specificity to full antibodies while having a smaller molecular size.

In some aspects, the capture agent can include an antibody or antibody fragment, an antigen, an aptamer, a peptide, a small molecule, a ligand, a molecular complex or any combination thereof. The capture agent can be any reagent that has specific binding affinity for different target biomarkers. In certain aspects, antibodies and antibody fragments can be derived from a human, rodent (e.g., mouse and rat), donkey, sheep, rabbit, goat, guinea pig, camelid, horse, or chicken. Various antibodies and antibody fragments can be designed to selectively bind a desired analyte. Representative biomarkers and capture agents for use in the systems and methods of the present disclosure are shown in Table 1 below.

TABLE 1
Electrochemical biosensor for the early prediction of disease.

| | | | | Electrochemical |
|---|---|---|---|---|
| Disease | Fluid | Biomarker | Antibodies | Methods |
| Breast cancer | Urine | MMP-9 | anti-MMP-9 | EIS, CV |
| | | ALP | anti-ALP | EIS, CV |
| | | BCA-225/BRST | anti-BCA-225 | EIS, CV |
| | | Haptoglobin | anti-Haptoglobin | EIS, CV |
| Periodontitis | Saliva | MMP9 | MMP9 ab | EIS, CV |
| Diabetes | Saliva | MMP9 | MMP9 ab | EIS, CV |
| | Saliva | IL6 | IL6 ab | EIS, CV |
| Oral Cancer | Saliva | MMP9 | MMP9 ab | EIS, CV |
| | Saliva | CYFRA21-1 | CYFRA21-1 ab | EIS, CV |
| Stroke | Saliva | MMP9 | MMP9 ab | EIS, CV |
| | Saliva | GSH | GSH ab | EIS, CV |
| | Serum | MMP 12 | MMP 12 ab | EIS, CV |
| Pancreatic | Serum | CA242 | CA242 ab | EIS, CV |
| Cancer | | | | |
| | | CA19-9 | CA19-9 ab | EIS, CV |
| | | CEA | CEA ab | EIS, CV |
| | | CA-125 | CA-125 ab | EIS, CV |
| Obesity | Serum | Adiponectin | Adiponectin Ab | EIS, CV |
| | Saliva | Adiponectin | Adiponectin Ab | EIS, CV |
| Cardiovascular- | Serum/ | BNP | Anti-BNP | EIS, CV |
| Atrial fibrillation | Saliva | | | |
| | | NT-proBNP | Anti-NT-proBNP | EIS, CV |
| Atherosclerosis | Serum/ | CRP | Anti-C Reactive | EIS, CV |
| | Saliva | | Protein | |
| | Serum | Cardiac troponin T | Anti-Cardiac | EIS, CV |
| | | | troponin T | |
| | | Myeloperoxidase | Anti- | EIS, CV |
| | | | myeloperoxidase | |
| | | Ox-LDL | Anti-Ox-LDL | EIS, CV |
| Alzheimer's | Saliva | Aβ₁₋₄₂ | anti-amyloid-β 1- | EIS, CV |
| | | | 42 antibody | |
| | | p-TAU | p-TAU Ab | EIS, CV |
| | | t-TAU | p-TAU Ab | EIS, CV |
| | | lactoferrin | Anti-Lactoferrin | EIS, CV |
| | | Acetylcholinesterase | Acetylcholinesterase | EIS, CV |
| | | | antibody | |
| Osteoarthritis | Serum/ | Cartilage | Anti- | EIS, CV |
| | Saliva | oligomeric matrix | COMP/Cartilage | |
| | | protein | oligomeric matrix | |
| | | | protein antibody | |
| Lung cancer | Serum | CYFRA21-1 | Anti-CYFRA21-1 | EIS, CV |
| | | Squamous cell | Anti-SCC-Ag | EIS, CV |
| | | carcinoma antigen | | |
| | | (SCC-Ag) | | |
| | | CA 125 | CA 125 Ab | EIS, CV |
| | | CEA | CEA ab | EIS, CV |
| | | Human Survivin | SURVIVIN Ab | EIS, CV |
| | | Recombinant | | |
| | | Protein | | |
| Oral cancer- | Serum | S100A7-Psoriasin | S100A7 Ab | EIS, CV |
| leukoplakia | | protein | | |
| Mental | Serum | BDNF Protein | BDNF | EIS, CV |
| disorders | | | Monoclonal | |
| | | | Antibody | |
| Periprosthetic | Serum | IL-6 | IL6 Ab | EIS, CV |
| Joint Infection | | | | |
| (PJI) | | | | |
| | | CRP | Anti-C Reactive | EIS, CV |
| | | | Protein | |
| Autism | Serum | BDNF Protein | BDNF | EIS, CV |
| | | | Monoclonal Ab | |
| | | IL-1β | IL-1β Ab | EIS, CV |
| Osteoporosis | Saliva | Osteocalcin | Anti-osteocalcin | EIS, CV |
| | | C-terminal | Anti-CTX | EIS, CV |
| | | telopeptide (CTX) | | |
| Peri Implantitis | Saliva | IL-1β | IL-1β Ab | EIS, CV |
| (dental) | | | | |
| | | IL-6 | IL6 Ab | EIS, CV |
| | | TNF-α | Anti TNF-α | EIS, CV |
| | | MMP-8 | Anti MMP-8 | EIS, CV |

The capture agent can be conjugated to the graphene oxide nanoparticles through various chemical linkage mechanisms. The conjugation of the capture agent to the graphene oxide nanoparticles can involve covalent bonding between functional groups on the capture agent and functional groups on the graphene oxide nanoparticles. The conjugation can provide stable attachment of the capture agent to the working electrode surface, allowing the capture agent to remain bound during contact with biological fluid samples.

When the capture agent is disposed on a surface of the working electrode, the capture agent can be oriented to expose binding sites for interaction with target biomarkers in the biological fluid. The orientation of the capture agent on the working electrode surface can affect the binding efficiency between the capture agent and the target biomarker. The capture agent disposed on the working electrode surface can retain binding activity for the target biomarker after conjugation to the graphene oxide nanoparticles.

The capture agent configured to selectively bind to a target biomarker associated with a disease can enable detection of disease-specific biomarkers in biological fluid samples. The selective binding of the capture agent to the target biomarker can result from the specific molecular recognition between the capture agent and the target biomarker. The selectivity of the capture agent can allow the electrochemical biosensor to distinguish between the target biomarker and other molecules present in the biological fluid sample.

In some cases, the antibody or antibody fragment used as the capture agent can be a monoclonal antibody. Monoclonal antibodies can provide consistent binding characteristics across multiple biosensor units due to the uniform binding site structure of monoclonal antibodies. In some cases, the antibody or antibody fragment used as the capture agent can be a polyclonal antibody. Polyclonal antibodies can recognize multiple epitopes on the target biomarker, which can provide binding to the target biomarker even if certain epitopes are blocked or modified.

The capture agent conjugated to the graphene oxide nanoparticles can be immobilized at a concentration selected to provide adequate binding capacity for target biomarkers in the biological fluid sample. The concentration of the capture agent on the working electrode surface can affect the sensitivity of the electrochemical biosensor for detecting target biomarkers. In some cases, the capture agent concentration can be optimized through antibody saturation experiments to determine the concentration that provides the highest change in electrochemical properties upon biomarker binding.

The electrochemical biosensor can further comprise an organic linker molecule conjugated to the graphene oxide nanoparticles. The organic linker molecule can be conjugated to the capture agent. The organic linker molecule can serve as a crosslinker to immobilize the capture agent on the working electrode surface. The organic linker molecule conjugated to the graphene oxide nanoparticles and to the capture agent can provide a stable chemical linkage between the graphene oxide nanoparticles and the capture agent. In some embodiments, an organic linker molecule can comprise thiol, carboxylic, etc. terminal ends.

The selection of linker molecules can be influenced by several factors including bond-stability, position of functional groups, pH, presence/absence of amine groups for interaction with antibody, surface charge etc. For example, the availability of different functional groups in linker molecules can enable the immobilization of antibody through stable covalent linkage, and the antibody-antigen interactions provide specificity for detection of target biomarkers. As described herein, binding of capture reagents and subsequent biomarkers to the working electrode leads to changes in the ion diffusion profile near the nanostructures and hence changes in electrical properties (capacitance, resistance, etc.). The electrochemical detection methods described herein include means to directly characterize the capture reagent-target biomarker interactions based on charge perturbations at the electrode-electrolyte interface.

In some cases, the organic linker molecule comprises dithiobis succinimidyl propionate. Dithiobis succinimidyl propionate (DSP) can be used as a crosslinker to immobilize antibodies on the electrode surface. DSP contains reactive N-hydroxysuccinimide ester groups that can react with primary amine groups present on antibodies and other proteins. The reaction between the N-hydroxysuccinimide ester groups of DSP and the primary amine groups of the capture agent can form stable amide bonds, resulting in covalent attachment of the capture agent to the working electrode surface.

The DSP can be dissolved in dimethyl sulfoxide (DMSO) to form a DSP solution for application to the working electrode surface. DMSO can serve as a solvent for DSP due to the solubility characteristics of DSP in organic solvents. The DSP solution can be applied to the working electrode surface prior to application of the capture agent to provide reactive sites for capture agent immobilization.

In some cases, the DSP solution concentration is 4.041 mg DSP per 1 mL DMSO. In some cases, the DSP solution concentration is 10 mM DSP dissolved in 1 mL DMSO. The DSP solution at these concentrations can provide an adequate number of reactive N-hydroxysuccinimide ester groups for immobilization of the capture agent on the working electrode surface. The concentration of DSP in the DSP solution can affect the density of capture agent immobilization on the working electrode surface.

The DSP solution can be applied to the working electrode surface after cleaning the working electrode and prior to application of the capture agent. The working electrode can be coated with the DSP solution to allow the DSP molecules to bind to the graphene oxide nanoparticles or to the gold electrode surface. After application of the DSP solution, the capture agent can be applied to the working electrode surface, where the capture agent can react with the N-hydroxysuccinimide ester groups of the DSP molecules to form covalent bonds between the capture agent and the working electrode surface.

The electrochemical biosensor can include a blocked surface to prevent nonspecific binding of molecules other than the target biomarker. After immobilization of the capture agent on the working electrode surface, the biosensor surface can be blocked with a blocking agent to occupy remaining binding sites on the working electrode surface that are not occupied by the capture agent. The blocking of the biosensor surface can reduce background signal caused by nonspecific adsorption of proteins and other molecules from the biological fluid sample onto the working electrode surface.

In some cases, the biosensor surface is blocked with TBS Superblock blocking buffer to prevent nonspecific binding after antibody immobilization. TBS Superblock blocking buffer can contain proprietary blocking agents that bind to unoccupied sites on the working electrode surface. The TBS Superblock blocking buffer can be applied to the working electrode surface after immobilization of the capture agent and allowed to incubate for a period of time sufficient to allow the blocking agents to bind to the working electrode surface. The TBS Superblock blocking buffer can provide effective blocking of nonspecific binding sites while maintaining the binding activity of the immobilized capture agent.

In some cases, the biosensor surface is blocked with bovine serum albumin (BSA) to prevent nonspecific binding. BSA can be used as an alternative blocking agent to TBS Superblock blocking buffer. BSA can adsorb to unoccupied sites on the working electrode surface and prevent nonspecific binding of molecules from the biological fluid sample. The BSA blocking agent can be applied to the working electrode surface as a solution in phosphate buffered saline (PBS) or another suitable buffer.

The biosensor electrode can be cleaned prior to use to remove contaminants from the electrode surface. In some cases, the biosensor electrode is cleaned with 70% isopropyl alcohol and deionized water before use. The cleaning protocol can involve applying 70% isopropyl alcohol to the electrode surface to remove organic contaminants, followed by rinsing with deionized water to remove residual isopropyl alcohol and water-soluble contaminants. The cleaning of the biosensor electrode with 70% isopropyl alcohol and deionized water can provide a clean electrode surface suitable for subsequent coating steps.

The biosensor can be washed with PBS between coating steps to remove unbound materials. After application of the DSP solution to the working electrode surface, the biosensor can be washed with PBS to remove unbound DSP molecules before application of the capture agent. After application of the capture agent to the working electrode surface, the biosensor can be washed with PBS to remove unbound capture agent molecules before application of the blocking agent. After application of the blocking agent to the working electrode surface, the biosensor can be washed with PBS to remove unbound blocking agent before contact with the biological fluid sample. The washing steps with PBS can remove materials that are not covalently bound or stably adsorbed to the working electrode surface, reducing background signal and improving the specificity of biomarker detection.

The electrochemical biosensor can be configured with a capture agent at a concentration determined through an antibody saturation process. The antibody saturation process can involve testing multiple antibody concentrations to identify the concentration that provides optimal binding characteristics for a particular target biomarker. The antibody saturation process can be performed by coating the working electrode with different concentrations of the capture agent and measuring the resulting electrochemical properties to determine which concentration provides the highest change in impedance or capacitance upon biomarker binding.

The antibody concentration for optimal saturation can vary depending on the target biomarker to be detected. Different antibodies can have different binding affinities and molecular sizes, which can affect the concentration at which the working electrode surface becomes saturated with capture agent molecules. The antibody saturation process can be performed for each type of capture agent to determine the concentration that provides adequate surface coverage without excess antibody that can reduce binding efficiency.

In some cases, the antibody concentration for optimal saturation is determined to be 2 micrograms per milliliter for CA19-9 detection. The anti-CA19-9 antibody at a concentration of 2 micrograms per milliliter can provide sufficient surface coverage on the working electrode for detection of CA19-9 biomarker in biological fluid samples. The 2 microgram per milliliter concentration of anti-CA19-9 antibody can be determined through antibody saturation experiments that measure changes in electrochemical properties at different antibody concentrations.

In some cases, the antibody concentration for optimal saturation is determined to be 1 microgram per milliliter for MMP-9 detection. The anti-MMP-9 antibody at a concentration of 1 microgram per milliliter can provide sufficient surface coverage on the working electrode for detection of MMP-9 biomarker in biological fluid samples. The 1 microgram per milliliter concentration of anti-MMP-9 antibody can result in the highest change in impedance and capacitance values during antibody saturation experiments, indicating full saturation of the electrode surface.

In some cases, the antibody concentration for optimal saturation is determined to be 7.5 micrograms per milliliter for GSH detection. The anti-GSH antibody at a concentration of 7.5 micrograms per milliliter can provide sufficient surface coverage on the working electrode for detection of GSH biomarker in biological fluid samples. The 7.5 microgram per milliliter concentration of anti-GSH antibody can be determined through electrochemical impedance spectroscopy modelling of change in impedance and percentage change in capacitance at different antibody concentrations.

In some cases, the antibody concentration for optimal saturation is determined to be 0.5 micrograms per milliliter for adiponectin detection. The anti-adiponectin antibody at a concentration of 0.5 micrograms per milliliter can provide sufficient surface coverage on the working electrode for detection of adiponectin biomarker in biological fluid samples. The 0.5 microgram per milliliter concentration of anti-adiponectin antibody can be determined through antibody saturation experiments that identify the concentration providing the highest impedance and capacitance values.

The biosensor can be incubated with an antibody solution for a period of time sufficient to allow the capture agent to bind to the working electrode surface. In some cases, the biosensor incubation time with antibody solution is 30 minutes at room temperature. The 30-minute incubation period can allow the antibody molecules to react with the organic linker molecules on the working electrode surface and form stable covalent bonds. The incubation at room temperature can provide suitable conditions for the reaction between the N-hydroxysuccinimide ester groups of the DSP molecules and the primary amine groups of the antibody molecules.

After immobilization of the capture agent and blocking of the biosensor surface, the biosensor can be contacted with a biological fluid sample containing the target biomarker. The biosensor can be incubated with the biological fluid sample for a period of time sufficient to allow the target biomarker to bind to the capture agent on the working electrode surface. In some cases, the biosensor incubation time with protein sample is 15-20 minutes. The 15-20 minute incubation period can allow the target biomarker molecules in the biological fluid sample to diffuse to the working electrode surface and bind to the capture agent molecules. The incubation time with the protein sample can be shorter than the incubation time with the antibody solution because the biomarker binding to the capture agent can occur more rapidly than the covalent attachment of the capture agent to the working electrode surface.

The electrochemical biosensor can include various antibodies as capture agents for detection of cancer-related biomarkers. As described above, the capture agent can comprise an antibody or an antibody fragment configured to selectively bind to a target biomarker associated with a disease. The antibody can be selected from the group consisting of anti-MMP-9, anti-CA19-9, anti-CEA, anti-CA242, anti-adiponectin, anti-ECM1, and anti-GSH. These antibodies can be used individually or in combination to detect biomarkers associated with various cancer types.

For breast cancer detection, the electrochemical biosensor can be configured with capture agents that selectively bind to breast cancer-associated biomarkers. In some cases, the capture agent comprises anti-ECM1 antibody for breast cancer detection. Extracellular matrix protein 1 (ECM1) can be overexpressed and present in the urine of breast cancer patients, making anti-ECM1 antibody a suitable capture agent for urine-based breast cancer detection. In some cases, the capture agent comprises anti-MMP-9 antibody for breast cancer detection. Matrix metalloproteinase 9 (MMP-9) can serve as a biomarker for breast cancer detection using urine samples.

In some cases, the capture agent comprises anti-BCA-225 antibody for breast cancer detection. The anti-BCA-225 antibody can selectively bind to BCA-225 antigen, which can be associated with breast cancer. In some cases, the capture agent comprises anti-haptoglobin antibody for breast cancer detection. The anti-haptoglobin antibody can selectively bind to haptoglobin protein, which can be present at altered levels in breast cancer patients. In some cases, the capture agent comprises anti-ALP antibody for breast cancer detection. The anti-ALP antibody can selectively bind to alkaline phosphatase (ALP), which can serve as a biomarker for breast cancer.

For pancreatic cancer detection, the electrochemical biosensor can be configured with capture agents that selectively bind to pancreatic cancer-associated biomarkers. In some cases, the capture agent comprises anti-CA19-9 antibody for pancreatic cancer detection. Carbohydrate antigen 19-9 (CA19-9) can be elevated in pancreatic cancer patients, with levels greater than 37 U/mL potentially indicating pancreatic cancer. In some cases, the capture agent comprises anti-CEA antibody for pancreatic cancer detection. Carcinoembryonic antigen (CEA) can be increased in 30% to 60% of patients with pancreatic cancer. In some cases, the capture agent comprises anti-CA242 antibody for pancreatic cancer detection. CA242 can provide a high level of sensitivity for pancreatic cancer detection compared to other biomarkers.

The biosensor can detect multiple biomarkers simultaneously including CA19-9, CA242, and CEA for pancreatic cancer detection. The simultaneous detection of multiple biomarkers can provide improved diagnostic accuracy compared to detection of a single biomarker. The electrochemical biosensor can include multiple working electrodes, each configured with a different capture agent for detection of a different biomarker. Alternatively, the electrochemical biosensor can be used sequentially with different capture agents to detect multiple biomarkers from the same biological fluid sample.

In some cases, the capture agent comprises anti-CA-125 antibody for pancreatic cancer detection. The anti-CA-125 antibody can selectively bind to cancer antigen 125 (CA-125), which can be associated with pancreatic cancer in addition to other cancer types.

For oral cancer detection, the electrochemical biosensor can be configured with capture agents that selectively bind to oral cancer-associated biomarkers. The disease detection can include oral cancer using salivary biomarkers. In some cases, the capture agent comprises anti-CYFRA21-1 antibody for oral cancer detection. The anti-CYFRA21-1 antibody can selectively bind to cytokeratin 19 fragment (CYFRA21-1), which can be present in saliva of oral cancer patients. In some cases, the capture agent comprises anti-S100A7 antibody for oral cancer-leukoplakia detection. The anti-S100A7 antibody can selectively bind to S100A7 protein, which can be associated with oral cancer and leukoplakia conditions.

For lung cancer detection, the electrochemical biosensor can be configured with capture agents that selectively bind to lung cancer-associated biomarkers. The disease detection can include lung cancer using serum biomarkers. In some cases, the capture agent comprises anti-pro-SFTPB antibody for lung cancer detection. The anti-pro-SFTPB antibody can selectively bind to pro-surfactant protein B (pro-SFTPB), which can be present at altered levels in serum of lung cancer patients. In some cases, the capture agent comprises anti-SCC-Ag antibody for lung cancer detection. The anti-SCC-Ag antibody can selectively bind to squamous cell carcinoma antigen (SCC-Ag), which can serve as a serum biomarker for lung cancer.

The selection of the antibody as the capture agent can depend on the target disease to be detected and the biological fluid sample to be analyzed. The antibody selected from the group consisting of anti-MMP-9, anti-CA19-9, anti-CEA, anti-CA242, anti-adiponectin, anti-ECM1, and anti-GSH can provide detection capabilities for various cancer types and other disease states. The additional antibodies including anti-BCA-225, anti-haptoglobin, anti-ALP, anti-CA-125, anti-CYFRA21-1, anti-S100A7, anti-pro-SFTPB, and anti-SCC-Ag can expand the range of detectable cancer types to include breast cancer, pancreatic cancer, oral cancer, and lung cancer.

In various aspects, the biological sample can be from a cancer patient, a patient suspected of having cancer, or a patient at risk of having cancer. The cancer can be, e.g., acute lymphoblastic leukemia (ALL), acute myeloid leukemia (AML), adrenocortical carcinoma, Kaposi Sarcoma, anal cancer, basal cell carcinoma, bile duct cancer, bladder cancer, bone cancer, osteosarcoma, malignant fibrous histiocytoma, brain stem glioma, brain cancer, craniopharyngioma, ependymoblastoma, ependymoma, medulloblastoma, medulloeptithelioma, pineal parenchymal tumor, breast cancer, bronchial tumor, Burkitt lymphoma, Non-Hodgkin lymphoma, carcinoid tumor, cervical cancer, chordoma, chronic lymphocytic leukemia (CLL), chronic myelogenous leukemia (CML), colon cancer, colorectal cancer, cutaneous T-cell lymphoma, ductal carcinoma in situ, endometrial cancer, esophageal cancer, Ewing Sarcoma, eye cancer, intraocular melanoma, retinoblastoma, fibrous histiocytoma, gallbladder cancer, gastric cancer, glioma, hairy cell leukemia, head and neck cancer, heart cancer, hepatocellular (liver) cancer, Hodgkin lymphoma, hypopharyngeal cancer, kidney cancer, laryngeal cancer, lip cancer, oral cavity cancer, lung cancer, non-small cell carcinoma, small cell carcinoma, melanoma, mouth cancer, myelodysplastic syndromes, multiple myeloma, medulloblastoma, nasal cavity cancer, paranasal sinus cancer, neuroblastoma, nasopharyngeal cancer, oral cancer, oropharyngeal cancer, osteosarcoma, ovarian cancer, pancreatic cancer, papillomatosis, paraganglioma, parathyroid cancer, penile cancer, pharyngeal cancer, pituitary tumor, plasma cell neoplasm, prostate cancer, rectal cancer, renal cell cancer, rhabdomyosarcoma, salivary gland cancer, Sezary syndrome, skin cancer, nonmelanoma, small intestine cancer, soft tissue sarcoma, squamous cell carcinoma, testicular cancer, throat cancer, thymoma, thyroid cancer, urethral cancer, uterine cancer, uterine sarcoma, vaginal cancer, vulvar cancer, Waldenstrom Macroglobulinemia, or Wilms Tumor. In an embodiment, the cancer can be pancreatic cancer. In another embodiment, the cancer can be breast cancer.

The electrochemical biosensor can include various antibodies as capture agents for detection of biomarkers associated with diseases other than cancer. The capture agent can comprise antibodies configured to selectively bind to biomarkers associated with obesity, stroke, diabetes, cardiovascular disease, Alzheimer's disease, and osteoarthritis.

For obesity detection, the electrochemical biosensor can be configured with capture agents that selectively bind to obesity-associated biomarkers. In some cases, the capture agent comprises anti-adiponectin antibody for obesity detection. Adiponectin can be secreted by mature adipocytes and can be present at altered levels in individuals with obesity. The anti-adiponectin antibody can selectively bind to adiponectin protein in biological fluid samples such as saliva or serum for detection of childhood obesity and other obesity-related conditions.

For stroke risk prediction, the electrochemical biosensor can be configured with capture agents that selectively bind to stroke-associated biomarkers. The biosensor can detect MMP-9 and glutathione (GSH) biomarkers together for stroke risk prediction. In some cases, the capture agent comprises anti-MMP-9 antibody for stroke detection. Matrix metalloproteinase 9 (MMP-9) can be implicated in blood brain barrier disruption and hemorrhagic transformation following ischemic stroke. In some cases, the capture agent comprises anti-GSH antibody for stroke detection. Glutathione (GSH) can inhibit oxidative stress and reduce MMP-9 levels to avoid pathological angiogenesis in ischemic stroke. The electrochemical properties of MMP-9 and GSH can have opposite effects in the artificial saliva system, which can relate to the negative effects of MMP-9 and positive effects of GSH on the cellular system. The combined detection of MMP-9 and GSH biomarkers can provide improved stroke risk prediction compared to detection of a single biomarker.

For diabetes detection, the electrochemical biosensor can be configured with capture agents that selectively bind to diabetes-associated biomarkers. In some cases, the capture agent comprises anti-IL6 antibody for diabetes detection. Interleukin-6 (IL-6) can be associated with inflammatory processes related to diabetes. The anti-IL6 antibody can selectively bind to IL-6 protein in biological fluid samples for detection of diabetes or diabetes risk.

For cardiovascular disease detection, the electrochemical biosensor can be configured with capture agents that selectively bind to cardiovascular disease-associated biomarkers. In some cases, the capture agent comprises anti-BNP antibody for cardiovascular disease detection. B-type natriuretic peptide (BNP) can be released by the heart in response to cardiac stress and can serve as a biomarker for cardiovascular disease. In some cases, the capture agent comprises anti-CRP antibody for cardiovascular disease detection. C-reactive protein (CRP) can be an inflammatory marker associated with cardiovascular disease risk. In some cases, the capture agent comprises anti-cardiac troponin 1 antibody for cardiovascular disease detection. Cardiac troponin I can be released into the bloodstream following cardiac muscle damage and can serve as a biomarker for myocardial infarction and other cardiovascular conditions. The electrochemical biosensor can include multiple capture agents for simultaneous detection of BNP, CRP, and cardiac troponin I to provide comprehensive cardiovascular disease risk assessment.

The ELISA analysis can be performed for each target biomarker to validate the biological activity of the capture agent and the detectability of the target biomarker. For MMP-9 detection, ELISA analysis can show increasing absorbance readings with increasing MMP-9 protein concentration. For GSH detection, ELISA analysis can show increasing absorbance readings with increasing GSH protein concentration. The ELISA results can confirm that the antibodies used as capture agents in the electrochemical biosensor are capable of binding to the target biomarkers with specificity.

The ELISA testing can provide a benchmark for evaluating the sensitivity of the electrochemical biosensor. The detection limit and dynamic range of the electrochemical biosensor can be compared to the detection limit and dynamic range of the ELISA method. The comparison can indicate whether the electrochemical biosensor provides comparable or improved sensitivity for biomarker detection compared to the established ELISA method.

The combination of scanning electron microscopy with energy dispersive spectroscopy, confocal microscopy imaging, and ELISA testing can provide comprehensive validation of the electrochemical biosensor. The SEM-EDS analysis can confirm surface modification and elemental composition changes associated with capture agent immobilization and biomarker binding. The confocal microscopy imaging can confirm biological activity of the antibody-antigen interaction on the electrode surface. The ELISA testing can validate the specificity and sensitivity of biomarker detection by the electrochemical biosensor. The validation methods can provide evidence that the electrochemical biosensor is capable of detecting target biomarkers through specific antibody-antigen interactions and that the electrochemical changes detected by the biosensor correlate with biomarker binding events.

The disease risk prediction system can be connected to a network for cloud-based machine learning model processing. The network can comprise a local area network, a wide area network, the Internet, or a combination of network types that allow the computing device to communicate with remote computing resources. The network connection can enable the computing device to transmit electrochemical data to cloud-based servers for processing by machine learning models hosted on the cloud-based servers.

Cloud-based machine learning model processing can involve transmitting the electrochemical data from the computing device to remote servers that execute the machine learning model. The remote servers can comprise cloud computing infrastructure that provides computational resources for executing machine learning algorithms. The cloud-based servers can host trained machine learning models including support vector machines and neural network models as described above. The computing device can transmit the electrochemical data to the cloud-based servers, and the cloud-based servers can process the electrochemical data using the machine learning model to determine the risk level associated with the disease. The cloud-based servers can transmit the risk level determination back to the computing device for presentation to a user.

Cloud-based machine learning model processing can provide advantages including access to computational resources that exceed the processing capabilities of the local computing device. The cloud-based servers can execute complex machine learning models that require substantial computational resources for inference operations. Cloud-based machine learning model processing can also allow for centralized updating of machine learning models, where updated model parameters can be deployed to the cloud-based servers without requiring updates to software on the local computing device.

The system can communicate via wireless protocols including Bluetooth, NFC, Wi-Fi, or cellular networks. The wireless communication protocols can enable data transfer between the electrochemical biosensor and the computing device, between the computing device and cloud-based servers, or between the computing device and other devices in a healthcare network.

Bluetooth communication can enable short-range wireless data transfer between the electrochemical biosensor and the computing device. Bluetooth can operate in the 2.4 GHz frequency band and can provide data transfer rates suitable for transmitting electrochemical data from the biosensor to the computing device. Bluetooth Low Energy (BLE) can be used to reduce power consumption during wireless communication, which can extend battery life in portable biosensor devices. The computing device can pair with the electrochemical biosensor using Bluetooth protocols to establish a secure wireless connection for data transfer.

Near field communication (NFC) can enable short-range wireless data transfer between the electrochemical biosensor and the computing device when the devices are placed in close proximity. NFC can operate at a frequency of 13.56 MHz and can provide data transfer over distances of a few centimeters. NFC communication can be used for initiating connections between the electrochemical biosensor and the computing device or for transferring small amounts of data such as device identification information or calibration parameters.

Wi-Fi communication can enable wireless data transfer between the computing device and network infrastructure including routers and access points that provide connectivity to the Internet and cloud-based servers. Wi-Fi can operate in the 2.4 GHz or 5 GHz frequency bands and can provide data transfer rates suitable for transmitting electrochemical data to cloud-based servers for machine learning model processing. The computing device can connect to Wi-Fi networks in clinical settings, home environments, or other locations where Wi-Fi infrastructure is available.

Cellular network communication can enable wireless data transfer between the computing device and cloud-based servers when Wi-Fi connectivity is not available. Cellular networks can include 4G LTE networks, 5G networks, or other cellular network technologies that provide mobile data connectivity. Cellular network communication can allow the disease risk prediction system to transmit electrochemical data to cloud-based servers and receive risk level determinations from locations where cellular network coverage is available. The computing device can include a cellular modem or can communicate with a smartphone or other cellular-enabled device to access cellular network connectivity.

The wireless communication protocols can be used individually or in combination to provide flexible connectivity options for the disease risk prediction system. In some cases, the computing device can use Bluetooth communication to receive electrochemical data from the electrochemical biosensor and Wi-Fi or cellular network communication to transmit the electrochemical data to cloud-based servers for machine learning model processing. The selection of wireless communication protocols can depend on the availability of network infrastructure, the data transfer requirements, and the power consumption constraints of the disease risk prediction system.

The electrochemical biosensor can be configured as a point-of-care device for home use by patients. The point-of-care device configuration can allow patients to perform biomarker detection and disease risk assessment in a home environment without requiring visits to clinical laboratories or healthcare facilities. The point-of-care device can be similar to a glucometer used by diabetic patients for blood glucose monitoring, providing a familiar form factor and user experience for patients performing self-testing.

The point-of-care device can comprise a compact housing that contains the electrochemical measurement circuitry, the computing device, and a display for presenting results to the user. The compact housing can be sized and shaped for handheld operation, allowing patients to hold the device in one hand while performing biomarker detection with the other hand. The point-of-care device can include a port or receptacle for receiving the electrochemical biosensor, where the biosensor can be inserted into the device for electrochemical measurements.

The point-of-care device can be battery-powered to allow for portable operation without connection to external power sources. The battery can be rechargeable or replaceable, and the point-of-care device can include power management circuitry to extend battery life during operation. The portable and battery-powered configuration of the point-of-care device can allow patients to perform biomarker detection at home, at work, or in other locations outside of clinical settings.

The disease risk prediction system can include a handheld hardware device with software for calculating change in impedance and matching with calibration curves. The handheld hardware device can comprise the computing device described above, including a processor and memory configured to execute machine-readable instructions for processing electrochemical data. The handheld hardware device can include electrochemical measurement circuitry, such as a potentiostat, integrated into the handheld housing for performing electrochemical impedance spectroscopy and cyclic voltammetry measurements on the electrochemical biosensor.

The electrochemical measurement circuitry can comprise hardware, software, or a combination of software and hardware. The electrochemical measurement circuitry can comprise a single or multiple microprocessors, field programmable gate arrays (FPGAs), or digital signal processors (DSPs). The electrochemical measurement circuitry can be electrically connected to an electrochemical biosensor. In some embodiments, the electrochemical measurement circuitry can be part of the electrochemical biosensor, for example the electrochemical measurement circuitry can be assembled or disposed on the substrate. Alternatively, the electrochemical measurement circuitry can be remote to the electrochemical biosensor.

In some embodiments, the electrochemical measurement circuitry can be connected to a computing device. The electrochemical measurement circuitry can be part of the computing device or can be separate. The computing device can be configured to process and/or display results obtained via the above-described electrochemical detection techniques. In some embodiments, the electrochemical signal response can be displayed on the computing device for further analysis or data manipulation by a user.

The software executed by the handheld hardware device can calculate the change in impedance resulting from biomarker binding to the capture agent on the working electrode surface. The software can receive raw impedance measurements from the electrochemical measurement circuitry and process the raw measurements to determine impedance values at specific frequencies as described above. The software can calculate the change in impedance by comparing the measured impedance values to baseline impedance values stored in the memory of the handheld hardware device.

The software can match the calculated change in impedance with calibration curves stored in the memory of the handheld hardware device. The calibration curves can represent the relationship between biomarker concentration and change in impedance established during calibration of the electrochemical biosensor with known concentrations of the target biomarker. The software can interpolate the calculated change in impedance on the calibration curve to determine the concentration of the target biomarker in the biological fluid sample. The software can use the determined biomarker concentration to assess the disease risk level for the patient.

The calibration curves can be specific to the target biomarker and the type of biological fluid sample being analyzed. The handheld hardware device can store multiple calibration curves for different biomarkers and different biological fluid types, allowing the device to be used for detection of various biomarkers associated with different diseases. The software can select the appropriate calibration curve based on user input indicating the type of test being performed or based on identification information encoded on the electrochemical biosensor.

The disease risk prediction system can include an indicator system with three colors to indicate biomarker levels and associated risk status. The indicator system can comprise a visual display element on the handheld hardware device that presents color-coded information to the patient regarding the results of the biomarker detection. The three-color indicator system can provide an intuitive and easily understood presentation of risk level information without requiring the patient to interpret numerical values or complex data.

The indicator system can include a green indicator to indicate normal biomarker levels. The green indicator can be displayed when the calculated change in impedance corresponds to a biomarker concentration within a normal range on the calibration curve. The green indicator can indicate to the patient that the detected biomarker level is within the range typically observed in healthy individuals and that the disease risk level is normal.

The indicator system can include an orange indicator to indicate pre-high biomarker levels. The orange indicator can be displayed when the calculated change in impedance corresponds to a biomarker concentration that is elevated above the normal range but below a high-risk threshold on the calibration curve. The orange indicator can indicate to the patient that the detected biomarker level is moderately elevated and that the patient can be at low risk or intermediate risk for the disease being assessed. The orange indicator can prompt the patient to consult with a healthcare provider for further evaluation or to perform follow-up testing.

The indicator system can include a red indicator to indicate high biomarker levels. The red indicator can be displayed when the calculated change in impedance corresponds to a biomarker concentration that exceeds a high-risk threshold on the calibration curve. The red indicator can indicate to the patient that the detected biomarker level is substantially elevated and that the patient can be at high risk for the disease being assessed. The red indicator can prompt the patient to seek medical attention for diagnostic evaluation and potential treatment.

The thresholds for the green, orange, and red indicators can be established based on clinical data correlating biomarker concentrations with disease states and risk levels. The thresholds can be stored in the memory of the handheld hardware device and can be specific to each target biomarker and disease type. The software can compare the determined biomarker concentration to the stored thresholds to select the appropriate color indicator for display to the patient.

The indicator system can comprise light-emitting diodes (LEDs) that illuminate in green, orange, or red colors based on the risk level determination. Alternatively, the indicator system can comprise a display screen that presents colored graphics or text indicating the risk level. The indicator system can be positioned on the handheld hardware device in a location that is visible to the patient during and after the biomarker detection process.

The point-of-care device can include additional features to guide patients through the biomarker detection process. The handheld hardware device can include a display screen that presents instructions for sample collection, biosensor insertion, and test initiation. The handheld hardware device can include audio output capability to provide spoken instructions or audible alerts to the patient. The handheld hardware device can include buttons or a touchscreen interface for patient input and navigation through device menus.

The point-of-care device configuration can allow patients to perform routine monitoring of biomarker levels associated with chronic conditions or disease risk factors. Patients can use the point-of-care device to track changes in biomarker levels over time, which can provide information regarding disease progression or response to treatment. The handheld hardware device can store historical test results in memory, allowing patients and healthcare providers to review trends in biomarker levels.

The disease risk prediction system can include a diagnostic kit configured for point-of-care testing of biological fluid samples. The diagnostic kit can comprise multiple components that together provide the materials and equipment for performing biomarker detection using the electrochemical biosensor. The diagnostic kit can be packaged as a self-contained unit that allows patients or healthcare providers to perform biomarker detection without requiring additional laboratory equipment or supplies.

The diagnostic kit can comprise a plate configured to hold biological fluid samples or to support the electrochemical biosensor during testing. The plate can comprise a multi-well plate with individual wells for containing biological fluid samples prior to application to the electrochemical biosensor. The plate can comprise a flat surface or tray that provides a stable platform for positioning the electrochemical biosensor during electrochemical measurements. The plate can be made of plastic, glass, or another material that is compatible with biological fluid samples and does not interfere with electrochemical measurements.

The diagnostic kit can comprise a pipette for transferring biological fluid samples. The pipette can be a disposable plastic pipette configured for single-use transfer of biological fluid samples from a collection container to the electrochemical biosensor or to wells in the plate. The pipette can be a calibrated pipette that dispenses a predetermined volume of biological fluid sample to ensure consistent sample volumes are applied to the electrochemical biosensor. The pipette can allow patients or healthcare providers to accurately transfer biological fluid samples without contamination or spillage.

The diagnostic kit can comprise handheld software for processing electrochemical data and determining disease risk levels. The handheld software can be provided as a software application installed on the handheld hardware device described above. The handheld software can comprise the machine-readable instructions that, when executed by the processor of the computing device, cause the computing device to determine electrochemical data, input the electrochemical data into the machine-learning model, and determine the risk level associated with the disease. The handheld software can be pre-installed on the handheld hardware device included in the diagnostic kit or can be provided as downloadable software that patients install on a smartphone or tablet computer.

The diagnostic kit can comprise artificial urine or another artificial biological fluid for calibration and testing purposes. The artificial urine can be formulated to simulate the ionic composition, pH, and osmolality of natural human urine as described above. The artificial biological fluid included in the diagnostic kit can be used to verify proper operation of the electrochemical biosensor prior to testing patient biological fluid samples. The artificial biological fluid can be used to establish baseline electrochemical measurements for comparison with measurements obtained from patient biological fluid samples. The diagnostic kit can include artificial saliva, artificial serum, or other artificial biological fluids depending on the target biomarker and disease type for which the diagnostic kit is configured.

The diagnostic kit can comprise a modified sensor configured for detection of a specific target biomarker. The modified sensor can comprise the electrochemical biosensor described above, including the working electrode with the capture agent conjugated to the graphene oxide nanoparticles. The modified sensor can be pre-coated with the capture agent specific to the target biomarker, such that the modified sensor is ready for use without requiring additional preparation steps by the patient or healthcare provider. The modified sensor can be packaged in a sealed container or pouch to maintain sterility and to protect the capture agent from degradation prior to use.

The diagnostic kit can include multiple modified sensors to allow for repeated testing or for testing of multiple biological fluid samples. The modified sensors included in the diagnostic kit can be individually packaged to maintain sterility and to prevent cross-contamination between sensors. The diagnostic kit can include modified sensors configured with different capture agents for detection of different target biomarkers, allowing the diagnostic kit to be used for assessment of multiple disease risk factors.

The diagnostic kit can include instructions for use that guide patients or healthcare providers through the biomarker detection process. The instructions for use can describe the steps for collecting biological fluid samples, preparing the electrochemical biosensor, performing electrochemical measurements, and interpreting the results. The instructions for use can include illustrations or diagrams that show the proper technique for each step of the testing process.

The diagnostic kit configuration can allow for point-of-care testing in clinical settings, home environments, or other locations outside of centralized laboratories. The self-contained nature of the diagnostic kit can reduce the need for specialized laboratory equipment and trained personnel for biomarker detection. The diagnostic kit can provide a convenient and accessible option for patients to monitor biomarker levels associated with disease risk factors.

The electrochemical biosensor, the computing device, and the machine learning model can operate together as an integrated system to detect biomarkers in biological fluid samples, process electrochemical data resulting from biomarker detection, and determine disease risk levels based on the processed electrochemical data. The integrated system can provide a workflow that begins with application of a biological fluid sample to the electrochemical biosensor and concludes with output of a risk level determination to a user.

The workflow of the integrated system can begin with preparation of the electrochemical biosensor for biomarker detection. The electrochemical biosensor can be removed from packaging and inspected to verify that the biosensor is undamaged and ready for use. The electrochemical biosensor can be inserted into a port or receptacle on the handheld hardware device or connected to a potentiostat that is communicatively coupled to the computing device. The computing device can detect the presence of the electrochemical biosensor and can initiate a startup sequence to prepare for electrochemical measurements.

The computing device can perform a baseline electrochemical measurement on the electrochemical biosensor prior to application of the biological fluid sample. The baseline electrochemical measurement can establish reference values for impedance, capacitance, and resistance of the electrochemical biosensor in the absence of the target biomarker. The baseline electrochemical measurement can be performed using electrochemical impedance spectroscopy at open circuit potential with an amplitude of 10 mV across the frequency range of 10,000 Hz to 0.01 Hz as described above. The computing device can store the baseline electrochemical data in memory for subsequent comparison with electrochemical data obtained after contact with the biological fluid sample.

Following the baseline electrochemical measurement, the biological fluid sample can be applied to the electrochemical biosensor. The biological fluid sample can be collected from a patient using appropriate collection techniques for the type of biological fluid being analyzed. Saliva samples can be collected by having the patient expectorate into a collection container or by using a swab to collect saliva from the oral cavity. Urine samples can be collected by having the patient urinate into a collection container. Serum or blood samples can be collected by venipuncture or fingerstick and can be processed to separate serum from cellular components if serum analysis is desired.

The biological fluid sample can be transferred to the electrochemical biosensor using a pipette or other sample transfer device. A predetermined volume of the biological fluid sample can be dispensed onto the working electrode of the electrochemical biosensor, where the biological fluid sample contacts the capture agent disposed on the working electrode surface. The biological fluid sample can be allowed to incubate on the electrochemical biosensor for a period of time sufficient to allow target biomarkers in the biological fluid sample to bind to the capture agent. The incubation time can be 15-20 minutes as described above, during which the target biomarker molecules diffuse to the working electrode surface and form complexes with the capture agent molecules.

After the incubation period, the computing device can initiate electrochemical measurements on the electrochemical biosensor to detect changes in electronic properties of the working electrode resulting from binding of the target biomarker to the capture agent. The computing device can control the potentiostat to perform electrochemical impedance spectroscopy measurements on the electrochemical biosensor. The electrochemical impedance spectroscopy measurements can be performed at open circuit potential with an amplitude of 10 mV across the frequency range of 10,000 Hz to 0.01 Hz. The computing device can also control the potentiostat to perform cyclic voltammetry measurements on the electrochemical biosensor to obtain additional electrochemical data.

The computing device can receive raw electrochemical measurement data from the potentiostat during the electrochemical measurements. The raw electrochemical measurement data can include voltage values, current values, and timing information from which impedance values can be calculated. The computing device can process the raw electrochemical measurement data to determine impedance magnitude and phase angle values at each measured frequency. The computing device can organize the processed impedance data into data structures suitable for subsequent analysis.

The computing device can fit the impedance data to the modified Randles circuit model to extract individual electrochemical parameters. The computing device can execute iterative optimization algorithms to adjust the circuit element values of the modified Randles circuit model to minimize the difference between the measured impedance data and the impedance predicted by the circuit model. The computing device can extract polarization resistance, solution resistance, and constant phase element values from the fitted modified Randles circuit model.

The computing device can calculate changes in electrochemical parameters by comparing the electrochemical data obtained after contact with the biological fluid sample to the baseline electrochemical data obtained prior to contact with the biological fluid sample. The computing device can calculate the change in impedance at the specific frequency of 0.0125 Hz or 0.0126 Hz by subtracting the baseline impedance value from the post-sample impedance value. The computing device can calculate the percentage change in capacitance by comparing the capacitance values extracted from the modified Randles circuit model fitting before and after contact with the biological fluid sample. The computing device can calculate changes in resistance and other electrochemical parameters using similar comparison operations.

The computing device can process cyclic voltammetry data to determine additional electrochemical parameters. The computing device can integrate the current response over the voltage sweep to calculate the cyclic voltammetry area. The computing device can identify peak current values and peak potential values from the cyclic voltammetry data. The cyclic voltammetry parameters can be combined with the impedance-derived parameters to form a comprehensive set of electrochemical data for input to the machine learning model.

The computing device can format the electrochemical data for input to the machine learning model. The electrochemical data can be organized as a feature vector comprising the change in impedance, the percentage change in capacitance, the change in resistance, the cyclic voltammetry area, and other electrochemical parameters. The feature vector can be normalized or scaled to match the format of the training data used to train the machine learning model. The computing device can apply data preprocessing operations to the feature vector to prepare the electrochemical data for analysis by the machine learning model.

The computing device can input the electrochemical data into the machine learning model for risk level determination. The machine learning model can be stored in the memory of the computing device as trained model parameters that define the decision boundaries between risk level categories. The computing device can execute the machine learning model by applying the trained model parameters to the electrochemical data to generate a risk level prediction. The support vector machine can classify the electrochemical data into one of the risk level categories of normal, low risk, or high risk based on the position of the electrochemical data relative to the decision boundaries learned during training.

The machine learning model can generate a confidence score or probability value associated with the risk level prediction. The confidence score can indicate the degree of certainty with which the machine learning model assigns the electrochemical data to a particular risk level category. Higher confidence scores can indicate that the electrochemical data is clearly associated with a particular risk level, while lower confidence scores can indicate that the electrochemical data falls near a decision boundary between risk level categories.

The computing device can determine the risk level associated with the disease based on the output of the machine learning model. The computing device can receive the risk level prediction and the associated confidence score from the machine learning model. The computing device can apply threshold criteria to the confidence score to determine whether the risk level prediction is sufficiently reliable for presentation to the user. If the confidence score exceeds a threshold value, the computing device can accept the risk level prediction as the final risk level determination. If the confidence score falls below the threshold value, the computing device can indicate to the user that the test result is inconclusive and that repeat testing can be warranted.

The computing device can output the risk level determination to the user through a display or indicator system. The computing device can activate the appropriate color indicator of the three-color indicator system based on the risk level determination. The green indicator can be activated for a normal risk level determination, the orange indicator can be activated for a low risk determination, and the red indicator can be activated for a high risk determination. The computing device can also display numerical values, text messages, or graphical representations of the risk level determination on a display screen of the handheld hardware device.

The computing device can store the risk level determination and associated electrochemical data in memory for subsequent retrieval and analysis. The stored data can include a timestamp indicating when the test was performed, the type of biological fluid sample analyzed, the target biomarker detected, the electrochemical parameters measured, and the risk level determination. The stored data can be used to track changes in biomarker levels and risk levels over time for a particular patient.

The computing device can transmit the risk level determination and associated electrochemical data to external systems through wired or wireless communication interfaces. The computing device can transmit the data to cloud-based servers for storage in a centralized database. The computing device can transmit the data to electronic health record systems used by healthcare providers. The computing device can transmit the data to smartphone applications that allow patients to view and track test results.

The integrated system can provide feedback to the user throughout the workflow to guide the user through the testing process and to indicate the status of the system at each stage. The computing device can display messages indicating when the electrochemical biosensor is detected and ready for use, when the baseline measurement is complete, when the biological fluid sample should be applied, when the incubation period is complete, when the electrochemical measurements are in progress, and when the risk level determination is available. The feedback provided by the integrated system can help users perform the testing process correctly and can reduce errors that could affect the accuracy of the risk level determination.

The integrated system can include error detection and handling capabilities to identify and respond to conditions that can affect the accuracy of the risk level determination. The computing device can monitor the electrochemical measurements for values that fall outside expected ranges, which can indicate problems with the electrochemical biosensor, the biological fluid sample, or the measurement equipment. The computing device can detect conditions such as insufficient sample volume, improper biosensor insertion, or electrical interference that can affect measurement quality. The computing device can alert the user to detected errors and can provide guidance for corrective actions.

The integrated system can support multiple testing modes for different target biomarkers and different disease types. The computing device can store multiple machine learning models trained for different biomarker-disease combinations. The user can select the appropriate testing mode based on the type of electrochemical biosensor being used and the disease risk being assessed. The computing device can load the appropriate machine learning model and calibration parameters for the selected testing mode. The integrated system can support testing for cancer biomarkers, stroke biomarkers, periodontitis biomarkers, diabetes biomarkers, cardiovascular disease biomarkers, and obesity biomarkers using the same hardware platform with different electrochemical biosensors and machine learning models.

The integrated system can operate in a standalone mode where all data processing and machine learning model execution occurs on the local computing device, or the integrated system can operate in a connected mode where electrochemical data is transmitted to cloud-based servers for processing. In standalone mode, the computing device can execute the machine learning model locally using trained model parameters stored in the memory of the computing device. In connected mode, the computing device can transmit the electrochemical data to cloud-based servers that execute the machine learning model and return the risk level determination to the computing device. The connected mode can allow for use of more complex machine learning models that require computational resources exceeding the capabilities of the local computing device.

In the following discussion, a general description of the system and its components is provided, followed by a discussion of the operation of the same. Although the following discussion provides illustrative examples of the operation of various components of the present disclosure, the use of the following illustrative examples does not exclude other implementations that are consistent with the principles disclosed by the following illustrative examples.

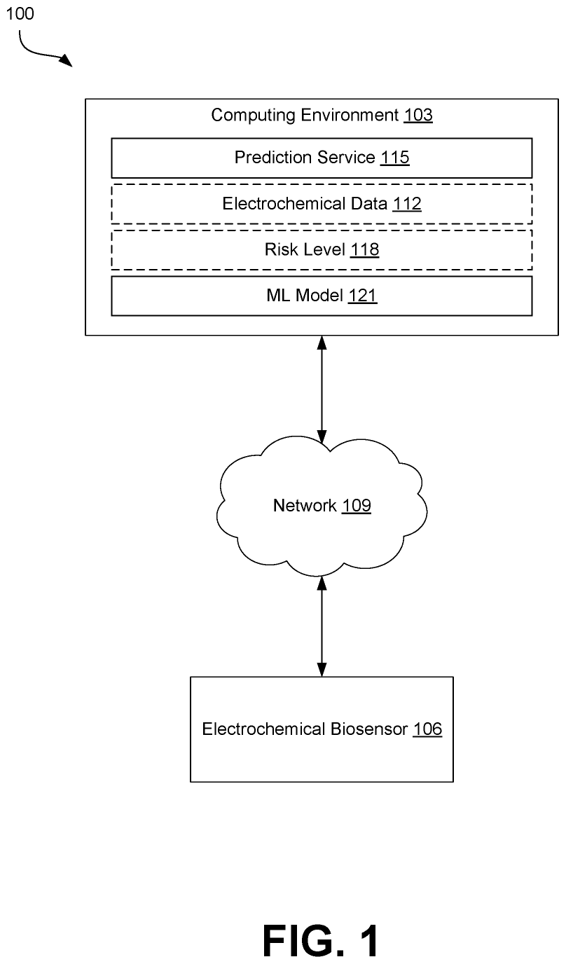

With reference to FIG. 1, shown is a network environment 100 according to various embodiments. The network environment 100 can include a computing environment 103, one or more electrochemical biosensors 106, which can be in data communication with each other via a network 109.

The computing environment 103 can include one or more computing devices that include a processor, a memory, and/or a network interface. For example, the computing devices can be configured to perform computations on behalf of other computing devices or applications. As another example, such computing devices can host and/or provide content to other computing devices in response to requests for content.

Moreover, the computing environment 103 can employ a plurality of computing devices that can be arranged in one or more server banks or computer banks or other arrangements. Such computing devices can be located in a single installation or can be distributed among many different geographical locations. For example, the computing environment 103 can include a plurality of computing devices that together can include a hosted computing resource, a grid computing resource or any other distributed computing arrangement. In some cases, the computing environment 103 can correspond to an elastic computing resource where the allotted capacity of processing, network, storage, or other computing-related resources can vary over time. In some examples, the computing environment 103 can include one or more displays, such as liquid crystal displays (LCDs), gas plasma-based flat panel displays, organic light emitting diode (OLED) displays, electrophoretic ink (“E-ink”) displays, projectors, or other types of display devices.

The electrochemical biosensor 106 can be representative of one or more of a plurality of electrochemical biosensors 106 that can be coupled to a network 109. The electrochemical biosensor 106 can include electrochemical measurement circuitry configured to detect and quantify a target analyte by converting a biologically specific interaction into an electrochemical signal. The electrochemical biosensor 106 can include a working electrode, a reference electrode, and a counter electrode, where the working electrode can comprise a capture agent on which a biorecognition element such as an enzyme, antibody, nucleic acid, or receptor is immobilized or otherwise associated. When a target analyte interacts with the capture agent, an electrochemical change occurs at the electrode surface, such as a change in current, voltage, charge, or impedance. The electrochemical measurement circuitry of the electrochemical biosensor 106 can be electronically coupled to the electrodes and controls the electrochemical conditions and measures the resulting signals. Accordingly, the electrochemical biosensor 106 can regulate an applied potential between electrodes, drive current through the system, measure resulting electrical responses, and condition the signal through amplification, filtering, and conversion into digital form. The electrochemical biosensor 106 can be further configured to interpret the measured signals to generate electrochemical data 112.

The network 109 can include wide area networks (WANs), local area networks (LANs), personal area networks (PANs), or a combination thereof. These networks can include wired or wireless components or a combination thereof. Wired networks can include Ethernet networks, cable networks, fiber optic networks, and telephone networks such as dial-up, digital subscriber line (DSL), and integrated services digital network (ISDN) networks. Wireless networks can include cellular networks, satellite networks, Institute of Electrical and Electronic Engineers (IEEE) 802.11 wireless networks (i.e., WI-FI®), BLUETOOTH® networks, microwave transmission networks, as well as other networks relying on radio broadcasts. The network 109 can also include a combination of two or more networks 109. Examples of networks 109 can include the Internet, intranets, extranets, virtual private networks (VPNs), and similar networks.

Electrochemical data 112 can include various data based at least in part on raw electrochemical signals measured by an electrochemical biosensor 106. In various examples, electrochemical data 112 can include a change in impedance, the percentage change in capacitance, the change in resistance, the cyclic voltammetry area, and other electrochemical parameters. In various examples, electrochemical data 112 can be derived from or generated by electrochemical signals measured by the electrochemical measurement circuitry of an electrochemical biosensor 106 and sent to a computing environment 103 for further processing.

Various applications or other functionality can be executed in the computing environment 103. The components executed on the computing environment 103 can include a prediction service 115, an ML model 121, and other applications, services, processes, systems, engines, or functionality not discussed in detail herein.

The prediction service 115 can be executed to receive electrochemical data 112 from one or more electrochemical biosensors 106 and output a resulting risk level 118. In some examples, the prediction service 115 can be executed to match the calculated change in impedance within electrochemical data 112 with calibration curves. The prediction service 115 can be executed to interpolate the calculated change in impedance on the calibration curve to determine the concentration of the target biomarker in the biological fluid sample. The prediction service 115 can then use the determined biomarker concentration to assess the disease risk level for the patient. For example, the prediction service 115 can be executed to input the electrochemical data 112 into a machine learning model 121 for risk level 118 determination.

A risk level 118 can represent a degree or magnitude of the likelihood that a particular event, such as the onset, progression, or complication of a disease, will occur within an individual. A risk level 118 can be quantitative or qualitative. For example, a risk level 118 can be “low”, “medium”, or “high”. In another example, a risk level 118 can be a probability score or percentage.

An ML model 121 can represent a model architecture (e.g., number and type of layers, nodes, parameters, etc.), a set of learned parameters (e.g., weights, biases, coefficients, etc.), and an inference algorithm that maps electrochemical data 112 to a risk level 118 prediction. An ML model 121 can be implemented as a neural network-based model, typically trained using self-supervised or unsupervised learning objectives (e.g., next-token prediction or masked token modeling) and optionally refined through supervised fine-tuning or reinforcement learning from human feedback (RLHF). In various examples, an ML model 121 can include architectures such as decoder-only, encoder-only, or encoder-decoder transformers, enabling tasks such as text generation, summarization, classification, reasoning, etc. In various examples, an ML model 121 can represent a large language model (LLM) or small language model (SLM), such as transformer-based architectures designed for natural language generation, reasoning, and comprehension tasks. In some examples, an ML model 121 can represent a supervised machine learning model 121 used for classification and regression tasks. For example, an ML model 121 can include a support vector machine (SVM). In some examples, the machine learning model 121 can be stored within the computing environment 103 as trained model parameters that define the decision boundaries between risk level 118 categories.

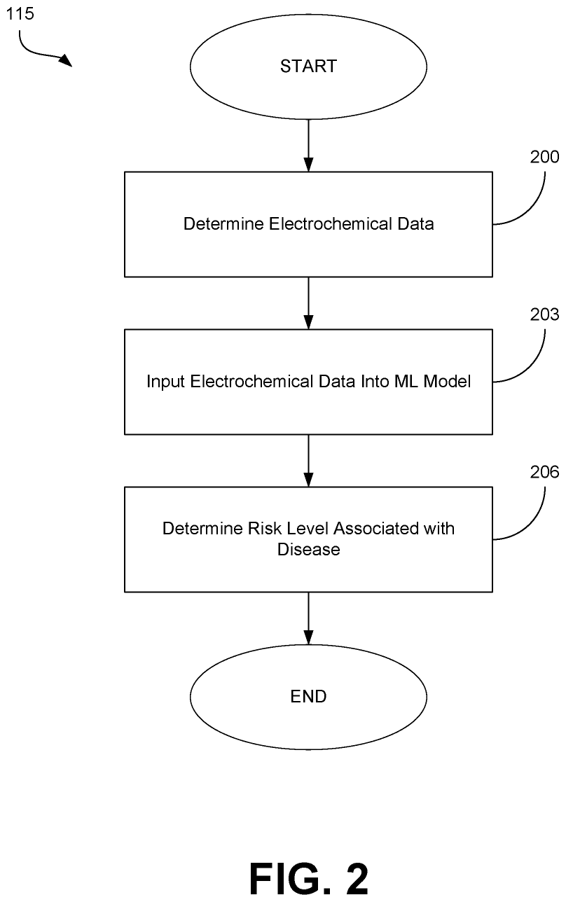

Referring next to FIG. 2, shown is a flowchart that provides one example of the operation of a portion of the prediction service 115. The flowchart of FIG. 2 provides merely an example of the many different types of functional arrangements that can be employed to implement the operation of the depicted portion of the prediction service 115. As an alternative, the flowchart of FIG. 2 can be viewed as depicting an example of elements of a method implemented within the network environment 100.

Beginning with block 200, the prediction service 115 can be executed to determine electrochemical data 112 based at least in part on one or more changes in an electronic property of a working electrode detected by the electrochemical biosensor 106. In some examples, the prediction service 115 can be executed to receive raw electrochemical measurement data from the electrochemical biosensor 106 during the electrochemical measurements. The prediction service 115 can process the raw electrochemical measurement data to calculate changes in electrochemical parameters by comparing the electrochemical data 112 obtained after contact with the biological fluid sample to the baseline electrochemical data 112 obtained prior to contact with the biological fluid sample.

At block 203, the prediction service 115 can then be executed to input the electrochemical data 112 into a machine-learning (ML) model 121 trained to analyze the electrochemical data 112 to determine a risk level 118 associated with the disease. In some examples, the prediction service 115 can be executed to apply the ML model 121 to the electrochemical data 112. In some examples, the prediction service 115 can be executed to call a function provided by a ML model 121 to input the electrochemical data 112 into the ML model 121 and provide the electrochemical data 112 as an argument to the function.

Then, at block 206, the prediction service 115 can be executed to determine the risk level 118 associated with the disease based at least in part on the ML model 121. In some examples, the prediction service 115 can be executed to receive a risk level 118 output by the ML model 121. In some examples, the prediction service 115 can be executed to determine a risk level 118 based at least in part on a risk level 118 and/or a confidence score output by the ML model 121 as described previously. After block 206, the process depicted by the flowchart of FIG. 2 can then end.

Examples: The following examples are provided solely to illustrate example implementations of the electrochemical biosensors, systems, and methods of use described herein and are not intended to limit the scope of the disclosure. In particular, any numeric values, thresholds, percentiles, time intervals, weights, scaling factors, reference values, and aggregation operators described in the examples are illustrative and can be varied. Other embodiments can use different ramp definitions, quantiles, exceedance thresholds, scenario sets, contingencies, constraint sets, normalization schemes, and combinations thereof, while remaining within the scope of the disclosure.

### Example 1—Stroke-Alert: Intelligent Salivary Biosensor Using Matrix Metallo-Proteinase 9 and Glutathione

Oral health as an indicator of systemic health is becoming increasingly relevant with many diseases like periodontitis linked to systemic diseases. One such systemic condition is stroke. According to the CDC, every 40 seconds someone in the United States has a stroke. Further, stroke is one of the leading causes of death for Americans.

Bodily fluids, such as saliva, can have molecules indicative of a disease condition, making the detection of biomarkers an up-and-coming field of early diagnosis. For example, MMP9 is a marker for periodontitis and is relevant in many systemic conditions. Further, GSH is a natural antioxidant in the body that helps to reduce oxidative stress.

In animal models of cerebral ischemia, matrix metalloproteinase (MMP) expression was significantly increased and was related to blood-brain barrier disruption, vasogenic edema formation, and hemorrhagic transformation. Matrix metalloproteinase-9 (MMP9) is a Zn2+-dependent endopeptidase that is found in many cell types throughout the body. Numerous studies have documented increases in MMPs, specifically MMP-9 levels, following stroke, with such perturbations associated with disruption of the blood-brain barrier (BBB), increased risk of hemorrhagic complications, and worsened outcomes. Glutathione (L-c-glutamyl-L-cysteinylglycine; GSH) is an intracellular thiol tripeptide present in all mammalian tissues that play a role in cellular protection against oxidant damage. GSH is reported to have a protective effect against brain vascular endothelial dysfunction, including endothelial barrier permeability, cell death, and the scavenging of ROS. Under normal physiological conditions of cellular homeostasis, glutathione remains primarily in its reduced form. However, many metabolic pathways involve oxidization of glutathione, resulting in an imbalance in cellular homeostasis. Impairment of glutathione function in the brain has been linked to loss of neurons during the aging process or as the result of neurological diseases such as Huntington's disease, Parkinson's disease, stroke, and Alzheimer's disease.

Accordingly, salivary redox biomolecules, including reduced glutathione, can be biomarkers in stroke diagnostics. This biomarker with high specificity and accuracy can distinguish stroke patients from controls but also correlate with cognitive impairment, as well as static and dynamic balance disorders. Importantly, this parameter does not depend on age, sex, or salivary gland function.

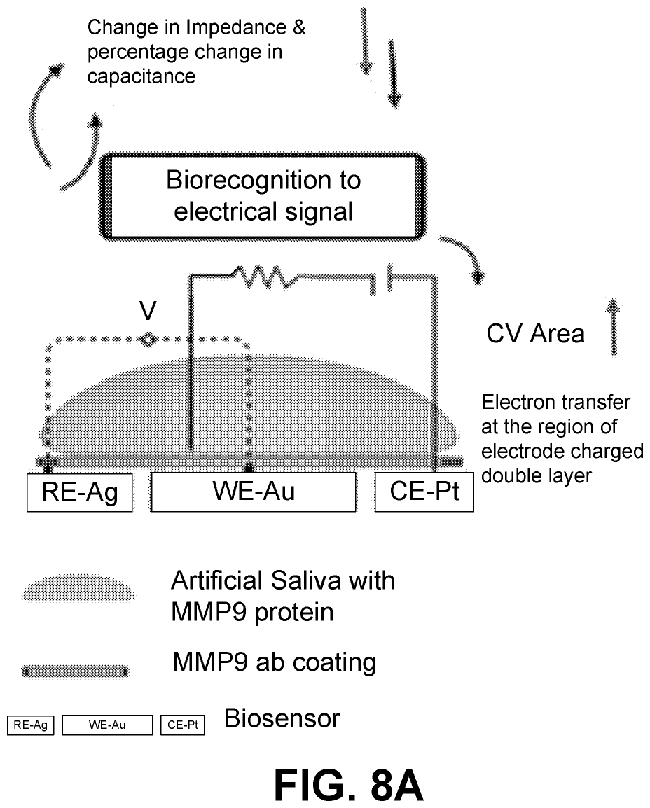

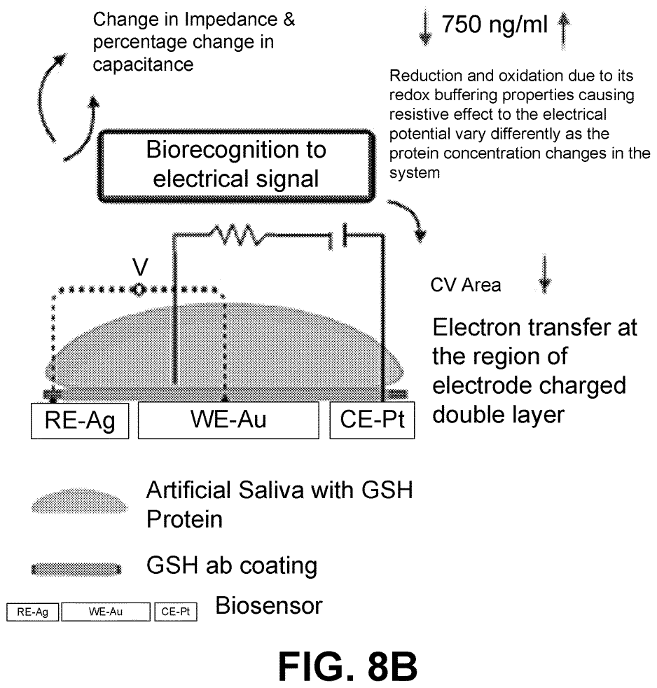

This Example considers periodontitis biomarker MMP9 and oxidative stress biomarker GSH to be used for the early risk prediction of stroke. Electrochemical analysis of artificial saliva was performed using a potentiostat with increasing concentrations of MMP9 and GSH protein independently. Electrochemical Impedance Spectroscopy (EIS) and Cyclic Voltammetry (CV) were then conducted. From the EIS data, the change in capacitance and charge transfer (CV area) was calculated for each test condition. The electrochemical properties obtained could be used together with the resistance of solution (Rsol), polarization resistance (Rp), and constant phase element (CPE) as machine learning data input. Scanning Electron Microscopy (SEM) with Energy Dispersive Spectroscopy (EDS) was employed to observe the change in the electrode surface and elemental composition data present on the sensor surface also showed the antibody is attaching to the biosensor enabling protein detection. ELISA and confocal imaging enabled biological characterization of MMP9 and GSH antibody-antigen interaction. Machine learning models using MMP9 and GSH electrochemical data were applied for the prediction of risk levels of stroke with periodontitis and salivary oxidative stress. The detection of MMP9 and GSH by electrochemical biosensors indicates the potential to use them as electrochemical biomarkers and use of the data for ML-driven prediction tool for stroke risk. For example, FIGS. 8A and 8B show biosensors for MMP9 protein (FIG. 8A) and GSH protein (FIG. 8B) in accordance with various aspects of the present disclosure.

TABLE 1
Concentrations of GSH protein (left) and MMP9 protein
(right) with associated stroke risk levels.

| | | | | Risk Level for |
|---|---|---|---|---|
| | | | | Periodontitis |
| | | Risk Level for | | with |
| Concentration | | Neurodegen- | Concentration | Neurodegen- |
| of GSH | | eration | of MMP9 | eration |
| 3 | μg/mL | Normal | Control | Normal |
| 1.5 | μg/mL | Low Risk | 500 ng/mL | Low Risk |
| 0.75 | μg/mL | Low Risk | 600 ng/mL | Low Risk |
| 0.5 | μg/mL | Low Risk | 700 ng/mL | Low Risk |
| 0.25 | μg/mL | High Risk | 750 ng/mL | High Risk |
| 0 | μg/mL | High Risk | | |

To generate a calibration study with varied concentrations of MMP9 and GSH protein, a biosensor was cleaned and DSP added. After 30 minutes, the DSP was aspirated and the antibody added. Following another 30 minutes, the antibody was aspirated and the biosensor was washed with PBS. A TBS Superblock was added and, after 30 minutes, subsequently aspirated. The biosensor was washed with PBS again, and the protein was added. After 15 minutes, the EIS and CV were run.

Once the biosensor was connected to a potentiostat, electrochemical tests, including EIS and CV, were run. A biological characterization was conducted using confocal microscopy and ELISA before scanning electron microscopy (SEM) imaging with energy dispersive x-ray spectroscopy (EDS) was performed in secondary electron mode. Various electrochemical parameters were then identified (e.g., CPE, Rp, Rsol, CV area, ΔZ) and a concentration of GSH protein was associated with a stroke risk level using ML. For example, a concentration of GSH of approximately 0.5 μg/mL can be associated with a low risk level for neurodegeneration.

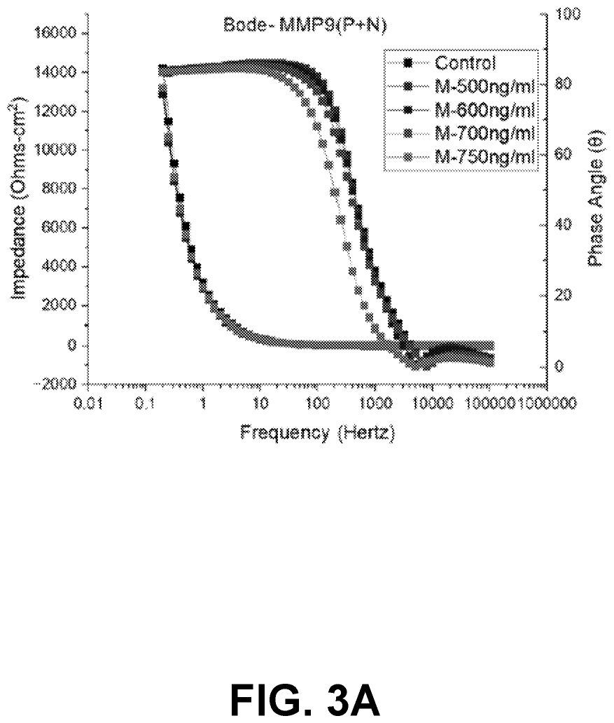

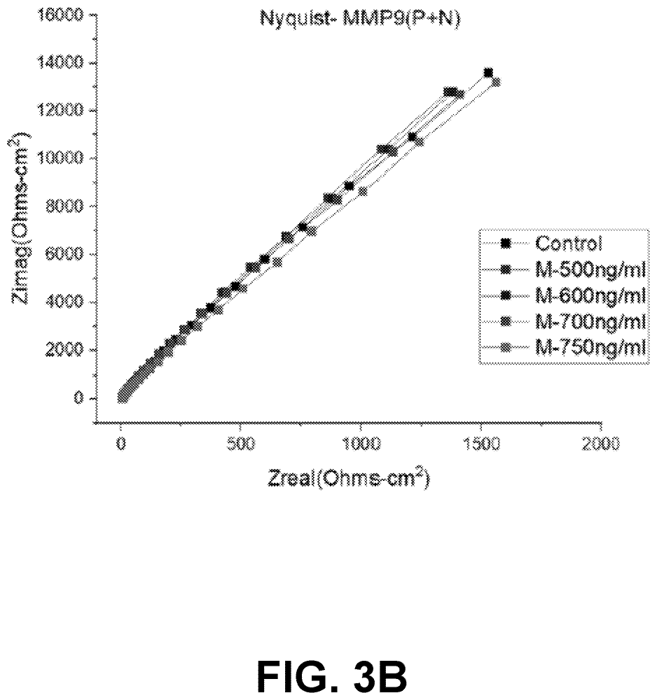

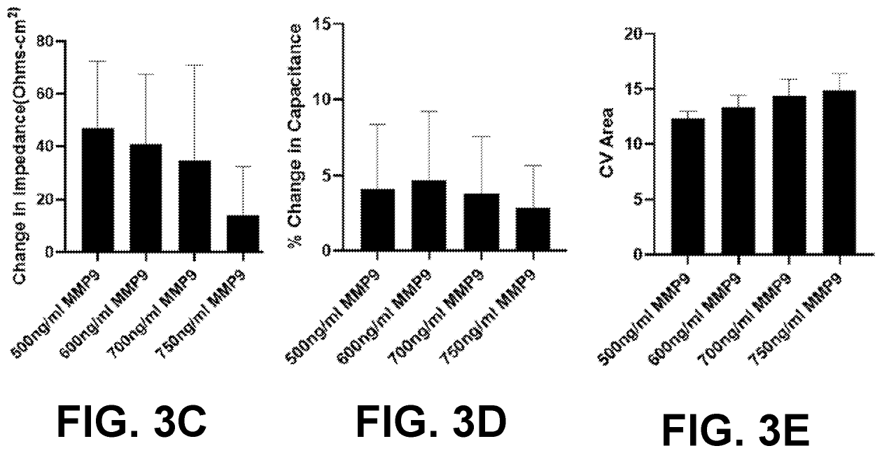

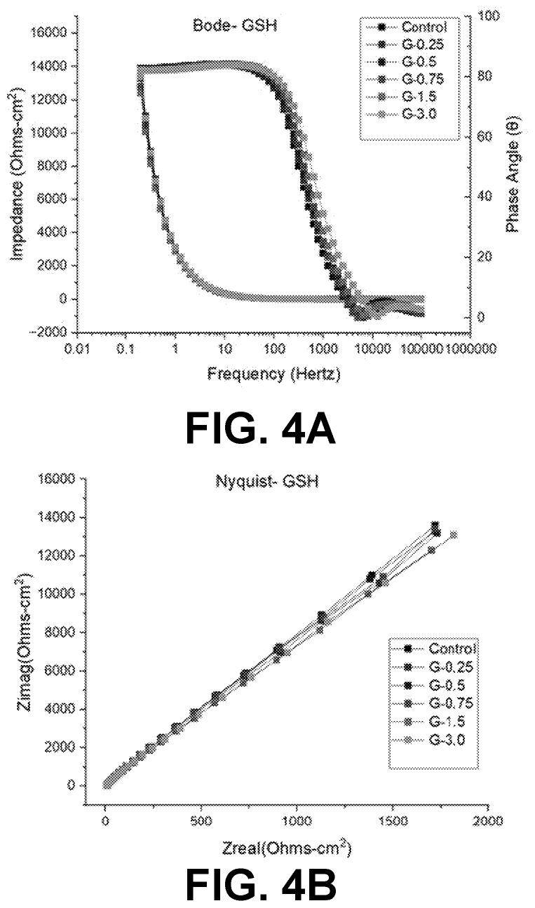

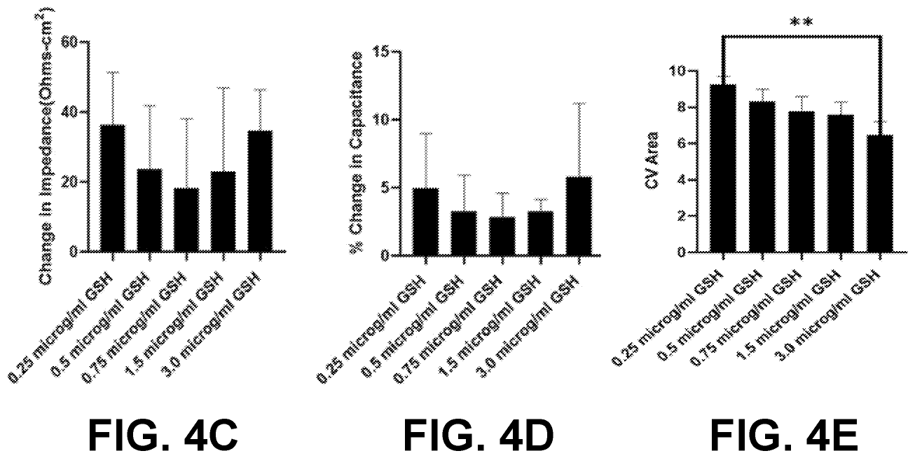

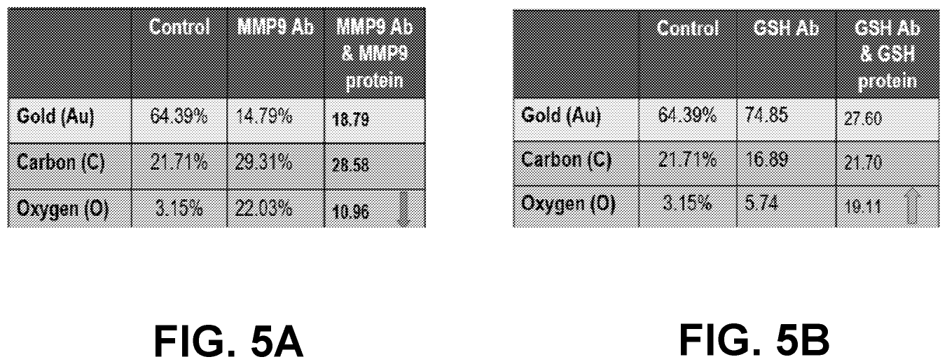

From the antibody saturation experiment data, electrochemical impedance spectroscopy modelling of change in impedance and percentage change in capacitance was obtained to be highest at 1 μg/mL for MMP9 antibody and 7.5 μg/mL for GSH antibody. From the protein calibration study, as the solution's protein concentration increased:
1. Change in impedance decreased with MMP9 concentration (FIG. 3C) and for GSH it decreased until 750 ng/mL and then increased (FIG. 4C).
2. Percentage change in capacitance decreased with MMP9 concentration (FIG. 3D) and for GSH it decreased until 750 ng/ml and then increased (FIG. 4D).
3. CV area showed an increasing trend with increasing MMP9 concentration (FIG. 3E) and a decreasing trend with increasing GSH concentration (FIG. 5E).

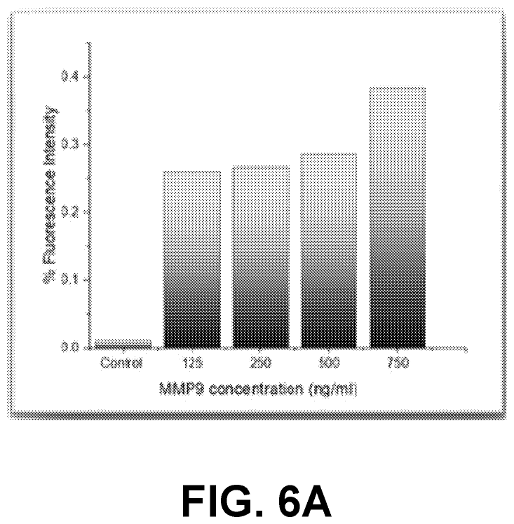

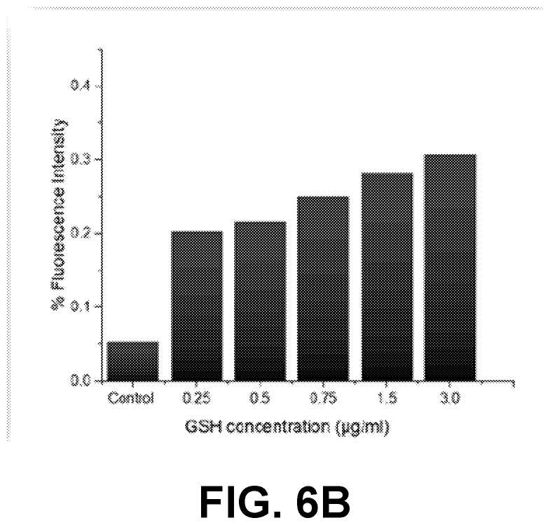

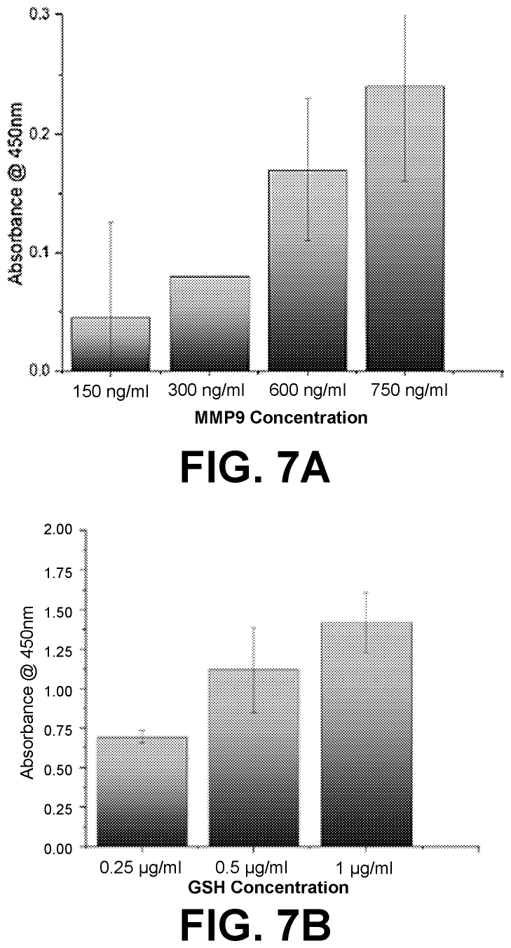

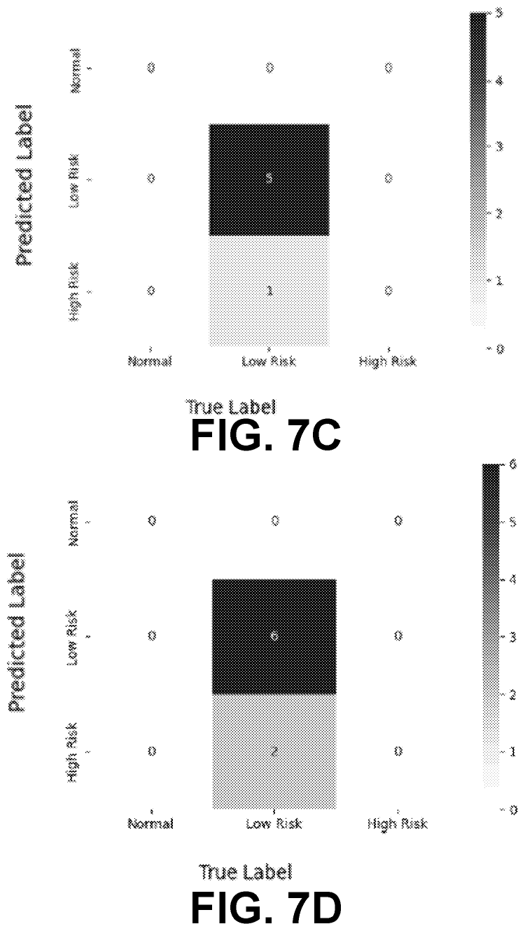

SEM EDS showed electrode surface being modified and elemental weight percentage (FIGS. 5A and 5B). Biological characterization of antibody and protein for MMP9 and GSH included (1) confocal imaging and percentage fluorescence analysis with Image J software indicated increasing % fluorescence with increasing protein concentration (FIGS. 6A and 6B), and (2) ELISA also gave the Absorbance reading increasing with increasing concentration of protein (FIGS. 7A & 7B). Electrochemical data with machine learning model (support vector machine) revealed that the SVM model gave an accuracy of 83.33% for MMP9 (FIG. 7C) and an accuracy of 75% for GSH (FIG. 7D).

The optimum concentration of antibody to be coated on the biosensors to be used for the protein electrochemical study was estimated. MMP9 protein calibration study and GSH protein calibration study showed that the electrochemical properties of the two proteins have opposite effects in the artificial saliva system which relates well to the negative effects of MMP9 and positive effects of the GSH on the cellular system. Biological characterization of antibody and protein by confocal imaging and percentage fluorescence analysis with Image J software as well as ELISA indicated that there is the antibody-antigen interaction of the protein and antibody used for biosensor study and it is relevant/useful for electrochemical testing. Scanning electron microscopy (SEM) with energy dispersive spectroscopy (EDS) employed to observe the change in the electrode surface and elemental composition data also indicates the antibody-antigen interaction is occurring on the biosensor surface enabling protein detection. Electrochemical data with a machine learning model-SVM gave the prediction possibilities and the use of machine learning for stroke risk prediction. Studies indicate GSH inhibits oxidative stress and reduces MMP9 levels in order to avoid pathological angiogenesis in ischemic stroke. The role of MMP9 and GSH in stroke and its correlation with salivary MMP9 and GSH will make them good markers for overall health and aging as well.

The detection of MMP9 and GSH protein by electrochemical biosensor indicates the potential to use MMP9 and GSH as salivary electrochemical biomarkers for stroke. The ML-driven prediction accuracy demonstrates the ability of risk prediction for the stroke using periodontitis marker MMP9 and oxidative stress marker GSH.

### Example 2—Early Prediction of Periodontitis with Saliva-Based Advanced Learning Biosensor

Periodontitis is marked by the inflammation and peeling away of the gum tissue, with bone and tooth loss in advanced stages. It affects nearly half of all Americans, including over 70 percent of the elderly. Periodontitis promotes the host immune response in the form of a complex network of molecular pathways involving proinflammatory mediators such as cytokines, growth factors, and MMPs. The enzyme MMP9 (matrix metalloproteinase 9) is a core component of the causal pathway behind periodontitis and comprises the most important pathway in tissue destruction associated with periodontal disease. MMPs participate in morphogenesis, physiological tissue turnover, and pathological tissue destruction. However, the alterations in the regulation of MMP activity are implicated in the manifestation of oral diseases. Moreover, MMPs can be considered a risk factor for periodontal disease. To that end, the measurements of MMP levels may be useful markers for early detection of periodontitis and as a tool to assess prognostic follow-ups.

Therefore, detection and inhibition of MMPs can be useful in periodontal disease prevention or be a part of periodontal disease therapy may greatly improve oral health globally. Electrochemical biosensors, combined with machine learning, can be viable for the early detection of periodontitis risk, using the biomarker MMP9.

### Example 3—Intelligent Biosensor for Urine-Based Early Diagnosis of Breast Cancer

Breast cancer is a significant health issue in the world today. As per 2020 breast cancer statistics, 2.26 million new cases and 685,000 deaths were reported worldwide. Procedures such as magnetic resonance imaging (MRI) and biopsies are commonly used to diagnose breast cancer. However, these techniques are very expensive, time-consuming, and painful for patients. A biosensor is a bioanalytical device that is used to detect analytes by converting them into an electrical signal to be analyzed in electronic form. Electrochemical biosensors can be capable of providing early and reliable diagnostic results. Early detection of cancer can greatly impact a patient's longevity. When breast cancer is diagnosed early, a patient's five year survival rate is 99%.

Intelligent biosensors for cancer can employ electrochemical techniques to identify biomarkers for the detection of breast cancer. While the concentrations of biomarkers increase in patient's urine, the detected electrochemical resistance also increases. Patients and physicians can monitor the resistance of urine as the early indicator for breast cancer. Serological diagnosis of breast cancer in the early stages can be conducted at home by patients using a biosensor of the present disclosure. Biosensors can also be used to monitor the effect of cancer treatments like chemotherapy or radiation. Accordingly, electrochemical biosensors can increase the chance of early detection of breast cancer and allow for improved clinical intervention. In order to simultaneously analyze multiple biomarkers, the obtained data can be analyzed by a machine-learning model, for which the diagnosis can be fast and accurately operated.

An intelligent biosensor for cancer can provide early breast cancer diagnosis in a point-of-care setting. It can be easier, faster, and more accurate for patients and can be utilized at home. The results can aid patients and physicians with early diagnosis. In various aspects of the present disclosure, an electrochemical biosensor can detect MMP9 and/or ECM-1, both of which are breast cancer protein biomarkers, in urine and an ML model can be utilized in early breast cancer diagnosis.

In this example, a study to detect various concentrations of MMP9 in phosphate-buffered saline (PBS) and artificial urine (pH=6.83) was first conducted. Then, a Python module to automate electrochemical impedance spectroscopy (EIS) raw data modeling and an ML algorithm for cancer risk prediction were developed. In the study, the electrodes of a sensor were cleaned with 70% isopropyl alcohol and DI water. Next, the entire sensor was coated with a DSP solution (4.041 mg DSP/1 mL DMSO), an antibody solution (5 μg antiMMP9/1 mL PBS or antiECM-1/PBS solution), and TBS Superblock. An MMP9 solution or an ECM-1 solution was then added to the sensor, which was analyzed through EIS and cyclic voltammetry (CV). Lastly, a Python script to model the sensor's equivalent circuit and a support vector machine (SVM) that classifies a patient's risk level into three categories were developed.

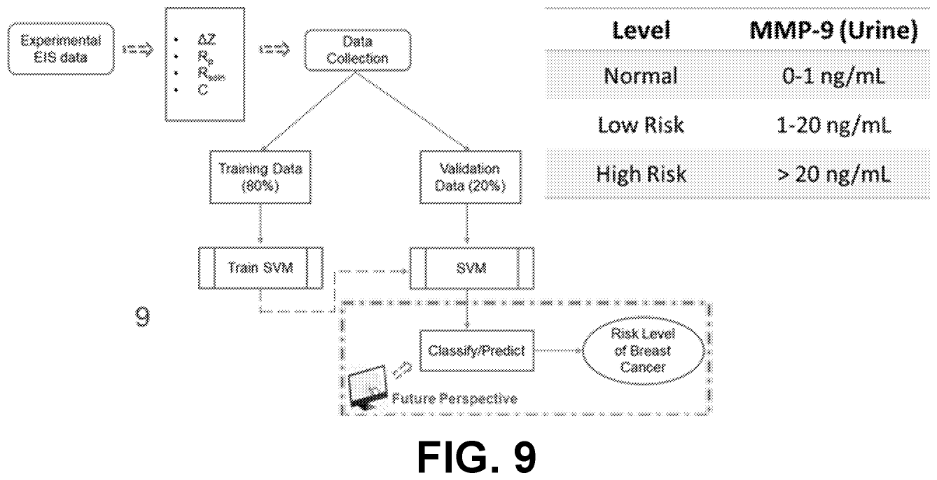

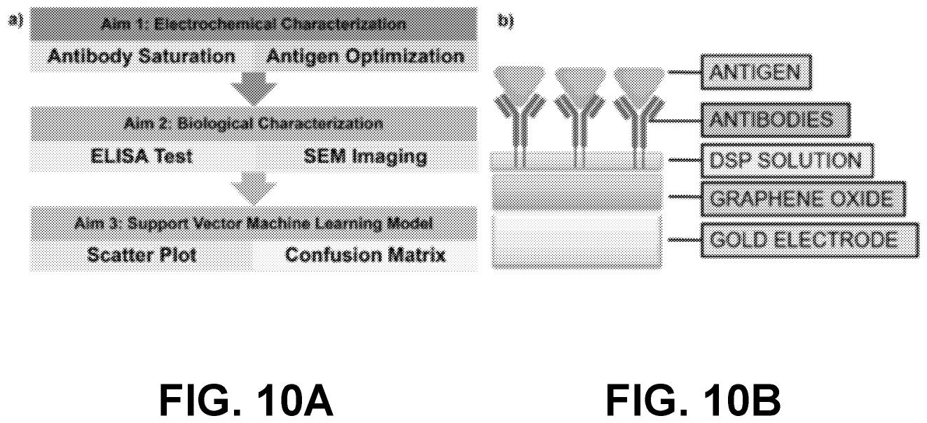

Using the Bode plot generated for each trial, the change in impedance (ΔZ) at a frequency of 0.0126 Hz was calculated. In artificial urine, an increase in ΔZ as MMP9 concentration increased was observed. Additionally, higher oxidation peaks for larger concentrations of MMP9 were found for PBS. When given the EIS and CV data for the MMP9 and extracellular matrix protein 1 (ECM-1) biomarkers, the SVM was observed to be about 84% accurate in the prediction of breast cancer risk. A linear increase in impedance is associated with an increase in ECM-1 concentration. As polymers of amino acids, both MMP9 and ECM-1 insulate electricity, contributing to a decrease in current and increase in impedance. The efficacy of SVMs in the analysis of EIS data was found in a previous study, in which a model accuracy of 98% was observed. In summary, MMP9 was successfully detected in artificial urine using biosensors, and ML was used to provide accurate risk predictions. FIG. 9 provides an overview of the risk level predictions. In some aspects, as shown in FIG. 9, electrochemical data can be split between training data and validation data to train a ML model and/or validate risk level predictions provided by the ML model.

TABLE 2
Concentrations of MMP-9 with associated risk levels.

| Level | MMP-9 (Urine) | |
|---|---|---|
| Normal | 0-1 | ng/mL |
| Low Risk | 1-20 | ng/mL |
| High Risk | >20 | ng/mL |

### Example 4—Development of Patient-Driven Diagnostic Tool for Early Breast Cancer Detection: Electrochemical Biosensor

Breast cancer is the most common cancer among females in the US and worldwide. It is the second highest cause of death among all cancers, accounting for approximately 571,000 deaths annually. Early diagnosis of breast cancer can lead to successful treatment and improve patient outcomes. Current techniques to detect breast cancers can include mammography, ultrasound, and biopsy. However, some of these methods can be expensive, painful, and unsuitable for some patients (e.g., time-consuming, low sensitivity, etc.). In addition, recently, investigators have used immunoassays techniques such as ELISA to detect biomarkers that indicate the presence of breast cancer. These techniques are often highly selective and sensitive, but such techniques can be high cost and prolonged.

Various embodiments of the present disclosure include a highly sensitive, economic, portable, and rapid method for breast cancer biomarker detection using a patient's urine. Moreover, various embodiments of the present disclosure include a biosensor as a point-of-care device that can be used at home by the patient. The ECM-1 biomarker can be used for breast cancer detection because high levels of ECM-1 expression was detected in breast cancer patients urine, as per the previous clinical reports. Thus, various aspects of the present disclosure provide an efficient alternative for clinical serological diagnosis of breast cancer and can be conducted at home by patients using an electrochemical biosensor to detect breast cancer biomarker ECM-1.

Accordingly, various aspects of the present disclosure include an electrochemical biosensor that immobilizes an antibody which binds to an antigen on the biosensor. Electrochemical testing (Electrochemical Impedance Spectroscopy-(EIS)) was then performed to test the immobilization of the antibody on the sensor and the affinity between the antibody and antigen. In addition, ELISA was performed to compare the detection range of the ECM-1 between ELISA and the biosensor.

In further aspects of the present disclosure, the ECM-1 protein can be calibrated using an electrochemical test for the electrochemical biosensor. Different concentrations of the ECM-1 protein were tested by electrochemical impedance spectroscopy to calibrate a standard curve for ECM-1 protein concentrations using the electrochemical biosensor. Accordingly, an electrochemical test and ELISA were performed.

In further aspects of the present disclosure, performance of the electrochemical biosensor was validated. Blind tests were performed to validate the electrochemical biosensor. Unknown concentrations of the ECM-1 were given to be tested by the electrochemical test. Then, the concentration was estimated from the standard curve of the ECM-1 in the urine. The results show that the detection range of ECM-1 in the urine solution from 1 pg/ml to 500 ng/ml. It had previously been reported that the serum level of ECM-1 protein in breast cancer patients is 10-12 ng/ml (normal level-0-5 ng/ml). Based on this, it was expected that the level of ECM-1 in the urine would be within the detection limit. This indicates that the sensor has the potential to identify breast cancer at an early stage of development or progressive recurrence with variation in the level of ECM-1 protein from the patient's urine.

### Introduction

### 1.1. Background

Breast cancer is a serious health concern for women. It is the most widespread cancer among females in the US and worldwide. It is the second cause of death among all cancers, accounting for 571,000 deaths annuallyr1. Each breast contains 15-20 glands called lobes, that are the milk-producing glands. These lobes are linked to the nipple by ducts. Breast cancer usually starts in the lobes. The breast also contains lymph nodes and vessels, so sometimes cancer can spread to other organs of the body through lymph nodes and vessels.

There are many causes of breast cancer; one being, genetic abnormality. 90% of breast cancers are caused by alterations in the genes or mistakes in the genetic materials. Another cause is having a previous history of breast cancer. A woman who had breast cancer before might have it again because the risk of getting breast cancer in the other breast will increase. Moreover, breast cancer is a genetic and inherited disease, and 5-10% of breast cancer because of inherited abnormalities.

The stages of breast cancer are defined by the size of the tumor and whether it has spread to the lymph nodes or other places in the body. There are various ways to stage breast cancer, but the most common way is from stage 0 to 4. Stage 0 is known as ductal carcinoma in situ (DCIS), and the cells are very few in the ducts and have not spread surrounding tissues. In stage 1, the size of the tumor is up to 2 cm, and it has not affected the lymph nodes. In stage 2, the size of the tumor is 2 cm, and it begins to spread to nearby lymph nodes. In stage 3, the size of the tumor is up to 5 cm, and it has spread to some lymph nodes. In stage 4, cancer spreads to a different part of the body, such as bones, liver, brain, or lungs.

### 1.2 Statement of Problem

Early diagnosis of breast cancer is very important because it can lead to successful treatment, and it increases the chance of a cure for cancer patients. For this aim, there are many methods available for cancer detection in the early stages, including mammography, biopsy, MRI, ultrasound, molecular breast imaging and thermography, etc.; moreover, there are biomarker-based expression techniques, such as, enzyme-linked immunosorbent assay (ELISA), radioimmunoassay (RIA) and immunohistochemistry (IHC). In addition, genomic detection methods such as polymerase chain reaction (PCR) and DNA sequencing are also used to detect cancer biomarkers. These techniques are efficient and effective because they can detect 80-90% of breast cancers in women; however, some of these methods are expensive, painful, require trained people, time-consuming and have low sensitivity.

### 1.3 Purpose

A new, highly sensitive, economic, portable and rapid technique was developed using an electrochemical biosensor for breast cancer biomarker detection from a patient's urine in the early stages as well as to monitor relapse after treatment. Moreover, a biosensor can be used as a point-of-care device, like a glucometer test for diabetic patients, at home by the patient or by normal women without clinical tests. There are several biomarkers identified for the detection of breast cancer. However, extracellular matrix 1 protein (ECM-1) biomarker was used for breast cancer detection because high levels of ECM-1 expression are detected in the urine of breast cancer patients.

### 1.4 Significance

Electrochemical biosensors can be an efficient and cost-effective alternative for clinical serological diagnosis of breast cancer in the early stages and can be conducted at home by patients. Moreover, women over 30 years old are more prone to have breast cancer, so a biosensor can be a helpful tool for them to do routine checkups for breast cancer and monitor their health frequently at home. Biosensors can also monitor the effect of cancer treatments like chemotherapy or radiation. Thus, breast cancer patients can use electrochemical biosensors after treatment of cancer. Use of electrochemical biosensors can increase the chance of early detection of breast cancer and better clinical intervention.

### Review of Current Methods

### 2.1 Methods of Breast Cancer Detection

There are many techniques available to detect breast cancer in the early stages because early breast cancer detection with suitable treatment can significantly increase the chance of curing cancer patients. Mammography is the most common method that is used to screen breast cancer. It is an x-ray of the breast, and it can detect any abnormality in the breast. However, mammography has low specificity and has less sensitivity for small tumors, which are less than 1 mm, and x-rays of the mammography can cause mutation in the tissues.

Another method to diagnose breast cancer is breast magnetic resonance imaging (MRI). It is a method that uses radio waves and magnets to take a picture of the internal breast. MRI can detect a small tumor that mammography cannot detect, but it is expensive, and it cannot detect some kinds of cancers like ductal and lobular carcinoma.

Biopsy is another method in which a small surgery is conducted to remove a sample of breast cancer to test whether the cells are cancerous or not. A biopsy can also differentiate between cancerous and benign tissues. On the other hand, the biopsy method can be expensive, painful and require trained medical personnel.

### 2.2 Biosensors

### 2.2.1 What is a Biosensor

A biosensor is a bioanalytical device that can be utilized to detect analytes by converting them into an electrical signal to be analyzed in an electronic form. In 1962, the first biosensor was introduced by Clark and Lyons and it was the first glucose biosensor to detect the level of glucose in the human body. Since then, biosensors have been improved to be an efficient detectable device for multiple applications in medical, industrial, and environmental fields.

Recently, biosensors were widely used in the medical field, especially for cancer detection in the early stages to provide better sensitivity and stability than other methods. Biosensors can be classified as point-of-care devices that can analyze a clinical sample at home, so patients can monitor their health by themselves.

### 2.2.2 The Structure of a Biosensor

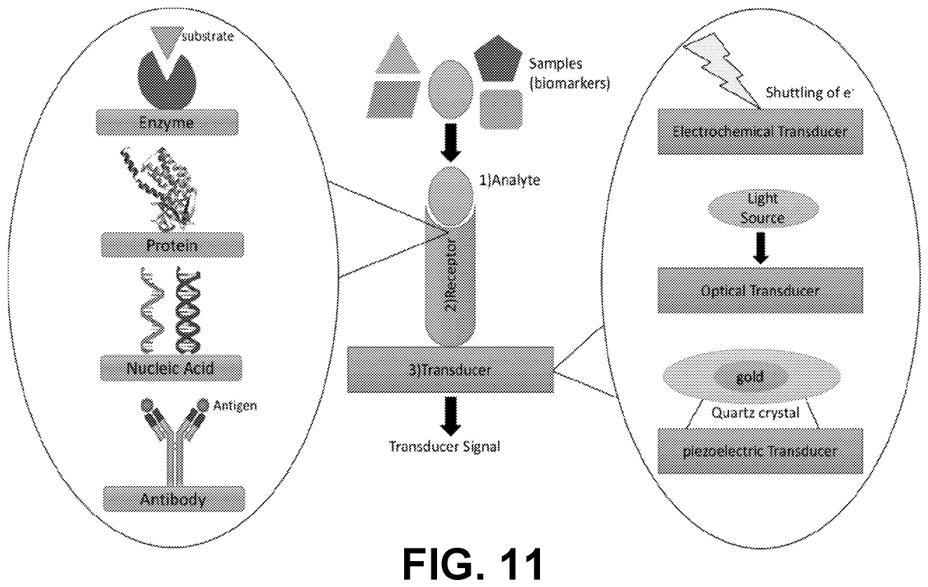

The biosensor can include two main parts which are receptor recognition elements and a transducer. Receptor recognition elements, such as nucleic acid, protein, enzyme, antibody or antigen, attract the analytes and convert them into electrical energy. The transducer is the device which is used to transform the electrical energy to a signal, such as electrochemical, acoustic, optical and thermal (calorimetric) transduction. FIG. 11 illustrates the principle of the biosensor. The electrochemical transducer makes use of potentiometric, amperometric or conductimetry/impedimentary principles. This transducer is generally used because it is easy, rapid, and cost effective. An optical transducer is based on reflectometry, and it is utilized in biosensors such as fluorescence, surface plasmon resonance (SPR) and spectroscopy of optical waveguides. A quartz crystal microbalance (QCM) is a device that measures the mass differences per unit area by measuring the frequency change of quartz crystal resonator. QCM devices are very common, especially in the medical field. Thermal transduction is a rare mechanism, and it is based on calorimetry that measures the change of temperature.

### 2.2.3 Types of Biosensors

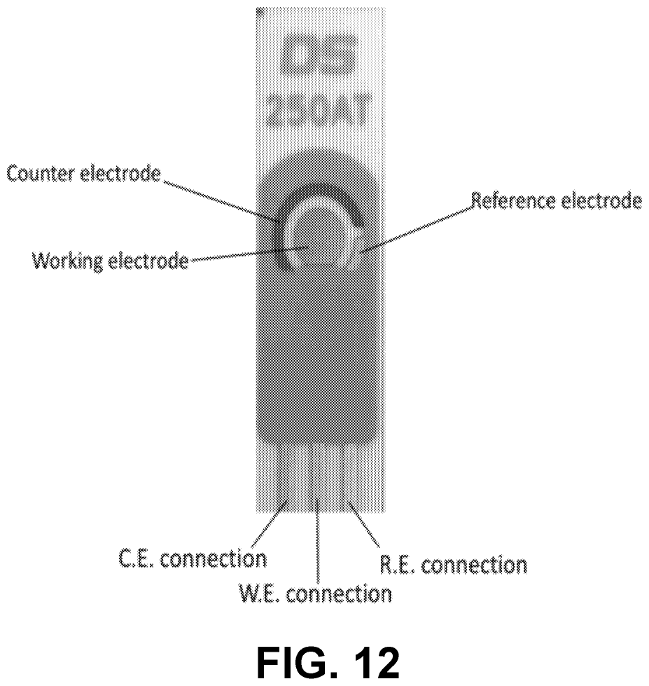

The electrochemical biosensor is widely used in the medical and bioengineering fields because it is inexpensive, rapid, easy to use and simple. Also, it is classified as a point-of-care device. Electrochemical biosensors transform biorecognition molecules and the biomarker interaction to electrochemical signals that can be measured (conductance, potential, impedance, current). The electrochemical transducer can include three electrodes, such as a working electrode (WE), a reference electrode (RE), and a counter electrode (CE). The interaction between the target molecule and bioreceptor occurs at the working electrode surface (gold area) (FIG. 12). Electrochemical biosensors use different techniques to obtain the readout, such as electrochemical impedance spectroscopy (EIS), stripping voltammetry (SWSV), cyclic voltammetry (CV), a field-effect transistor (FET), or square wave voltammograms (SWV). Currently, EIS gives awareness to bimolecular interactions out of their impact on electron transfer resistance (Ret). In addition, there are different nanomaterials recently used in electrochemical biosensors, including graphene oxide (GO), gold nanoparticle (AuNP), multi-walled carbon nanotubes (MWCNTs), GO with MWCNTs, and GO with gold nanoparticle to enhance the efficiency of the method. GO is the most common nanomaterial to use because it has a high sensitivity and affinity for biochemical material.

The optical biosensor is a light emission/absorption-based sensor which measures the alteration in the wavelength of light. The change in wavelength is measured by the optical biosensor. Many different optical biosensors have been developed for cancers detection in the early stages. There are different types of optical biosensors.

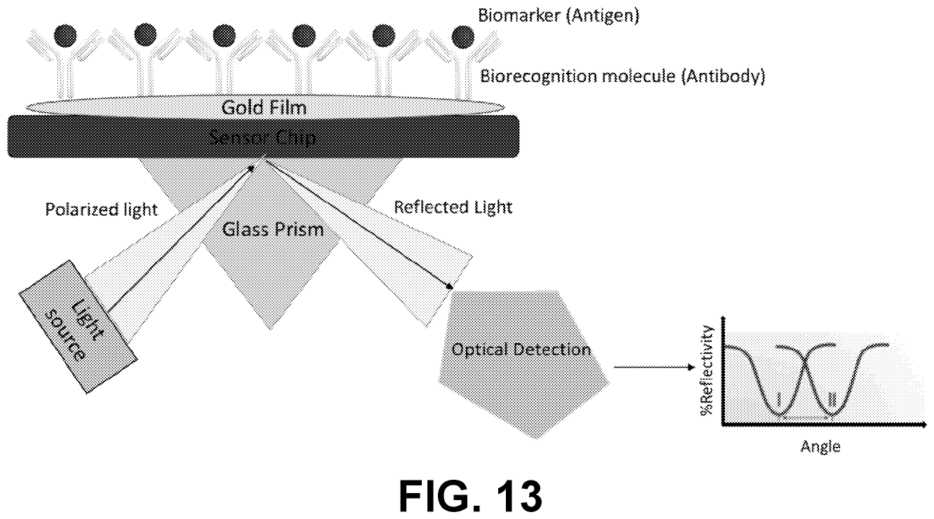

Surface plasmon resonance-based biosensor is an optical method that is based on the detection of the biomarker at the surface of the metal (typically gold). Biorecognition molecules such as an antibody are immobilized on the surface of the metal, then they will interact with the biomarker. This interaction results in a change in the refractive index and the mass of the sensing medium on the metal, and it causes a shift in the angle of SPR (FIG. 13). In addition, some optical biosensors utilize localized surface plasmon resonance (LSPR). LSPR happens when the surface plasmon gets excited by the light on the surface of the gold that causes the creation of scattering peak and spectral absorption.

Fluorescence is a kind of luminescence light that is emitted from molecules following the absorption of light. The fluorophore is a molecule that can absorb light at a shorter wavelength to emit it at a longer wavelength. One of the most significant fluorescence-based biosensors is fluorescence resonance energy transfer (FRET). It is non-radiative energy transfer between an excitation fluorophore (donor) and an absorption fluorophore (acceptor). The distance between them is very short which is 10-100 Å. FRET-based biosensor usually utilizes nanoparticles like quantum dots (QD) because it is most common for cancer detection. Moreover, it has high fluorescent for semiconducting nanoparticles, and the surface of QD forms bioconjugate with antibodies easily. Also, QD has its unique properties that can resist photo blinking and photobleaching.

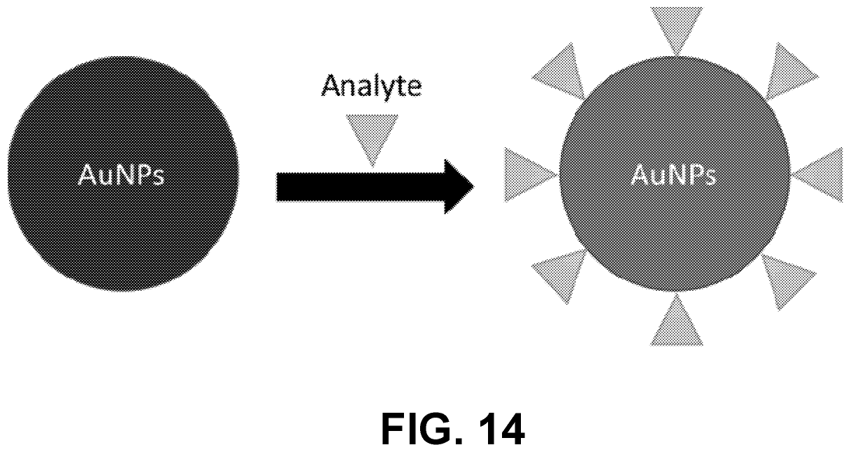

The colorimetric biosensor is based on color change on gold (AuNPs) when an analyte is present or absent. It is a simple and rapid operation for cancer detection (FIG. 14). AuNPs are most common to use in the colorimetric biosensor as a color development because of their property of rapid color changing that can give visual signal quickly.

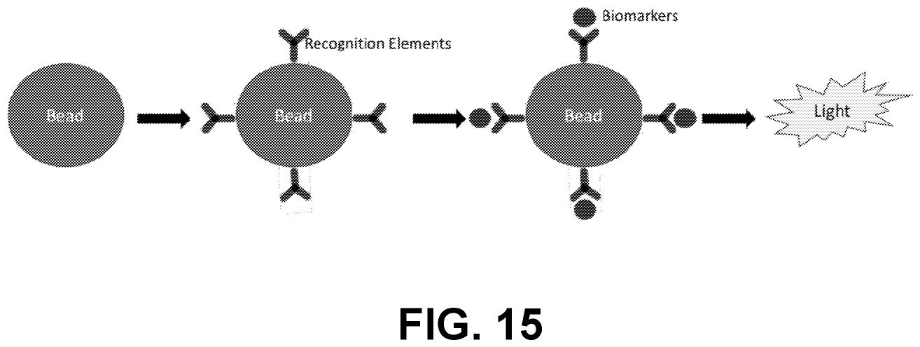

Luminescence is light emission when an excited electron of a molecule is coming back to the ground state. While an electron is in the excited state, chemiluminescence happens in the chemical reactions. Chemical luminescence-based biosensors measure the light emitted by a bio-chemiluminescence, and it is based on absorption of light. Light emission occurs when biomarkers bind to a recognition element such as antibodies (FIG. 15). The advantages of the chemiluminescence-based biosensor are high detectability, and simple measurement tool. The photons are generated by a chemical reaction, then they are measured effectively and easily without nonspecific signals.

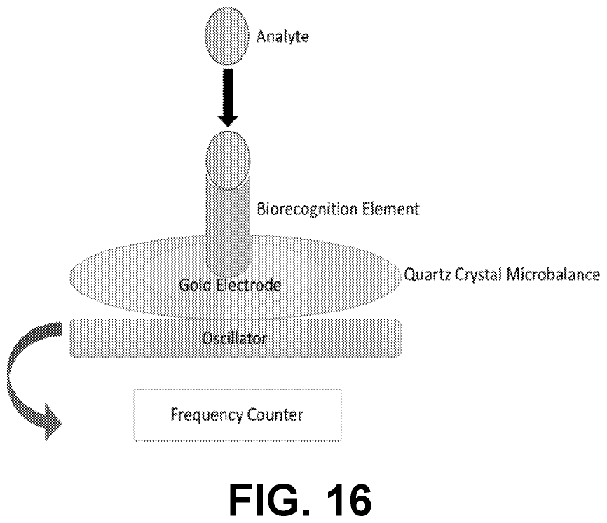

The piezoelectric biosensor uses a quartz crystal microbalance (QCM) device that measures the mass change and the frequency change of quartz crystal resonators based on adsorbate recognition (FIG. 16). Quartz is the most common and suitable crystal for analytical applications such as electrical, mechanical and chemical applications of quartz. QCM can detect small objects such as protein, nucleic acid, viruses, bacteria, and cells. The fabrication of piezoelectric biosensor is done by vapor deposition of silver or gold that serve as an electrode. The piezoelectric biosensor is popular in the medical field because it is easy to make rapid analysis, economic, and highly sensitive. Generally, piezoelectric immunosensors (sandwich immunoassay) are utilized to detect cancer biomarkers by immobilizing the antibodies on the surface of the sensor chip.

### 2.3 Electrochemical Impedance Spectroscopy (EIS)

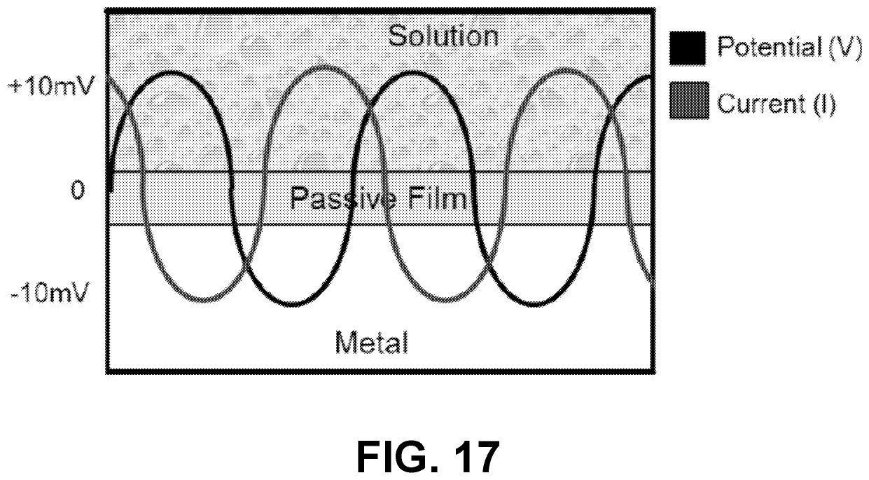

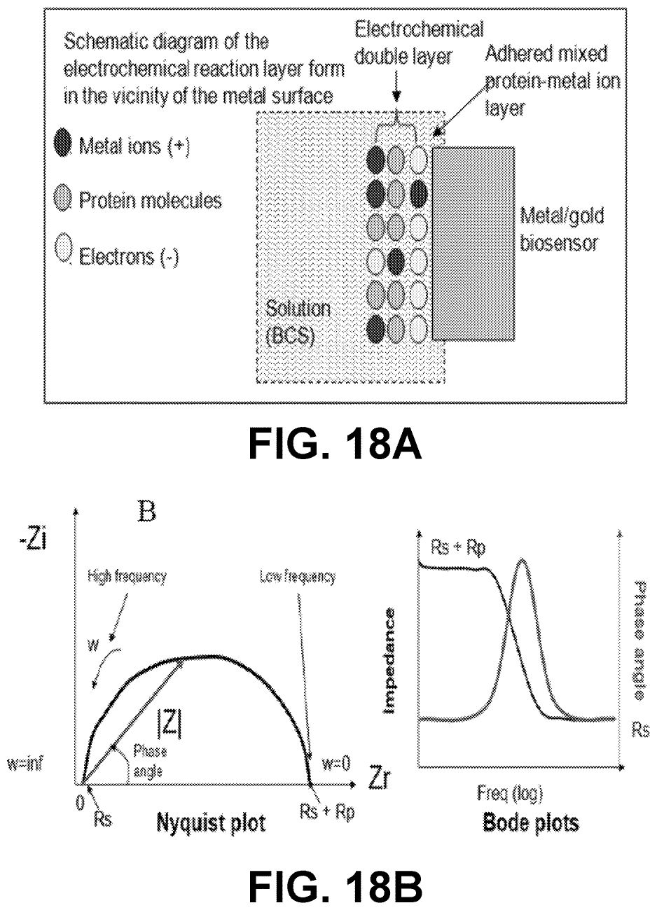

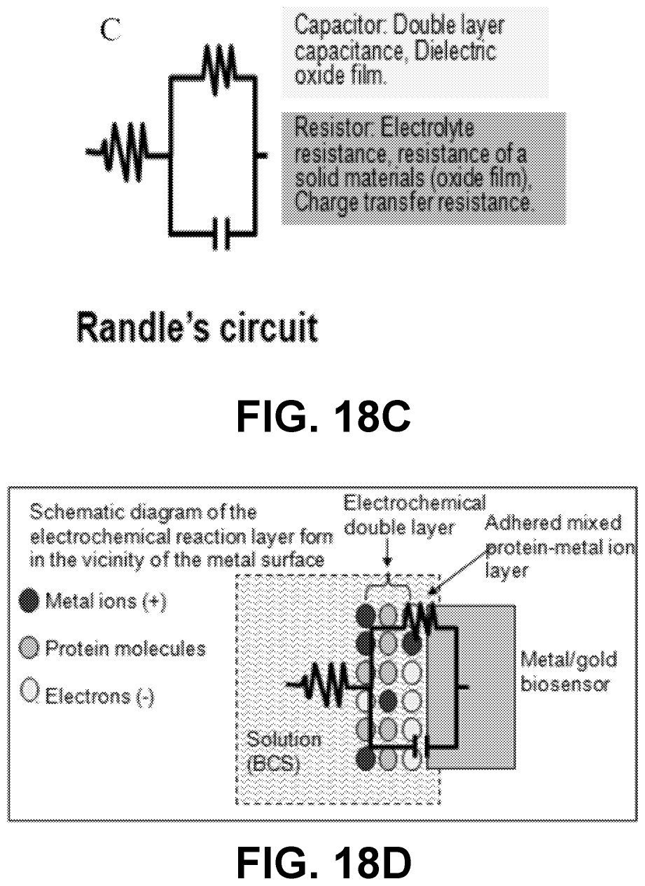

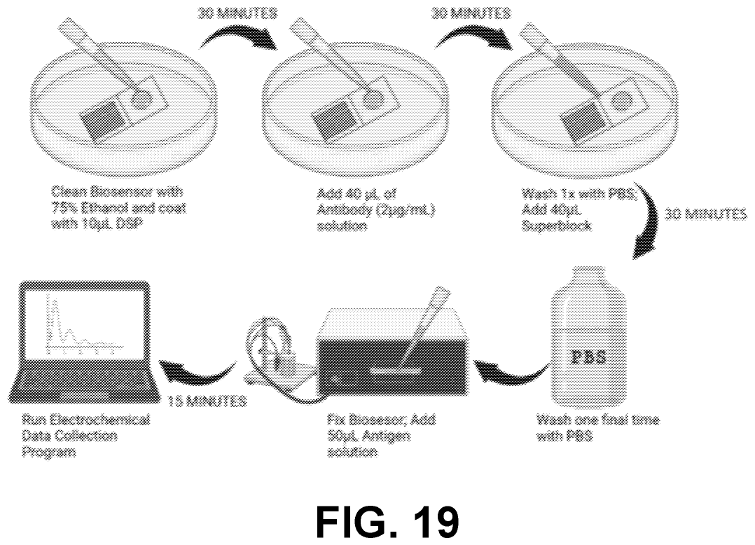

Electrochemical impedance spectroscopy (EIS) is a standard electrochemical technique, based on AC voltage. EIS is a useful experimental technique for the understanding of the corrosion kinetics of an electrochemical system. This technique is usually performed by applying a small excitation signal, such as a sinusoidal voltage (AC potential) of +10 mV across a spectrum of frequencies. Then, the current is measured in response to the applied voltage. A sinusoidal voltage will create a sinusoidal current at the same frequency but shifted in phase (FIG. 17). The impedance of the system will determine the phase shift. This impedance is frequency dependent and can elucidate the complex properties of the system such as energy storage (Capacitance) and the dissipation properties (Resistance). The obtained data can be depicted graphically by Bode or Nyquist plots (Shown in later sections). In addition, the data could be used to create an equivalent circuit model, to model the system response across the spectrum of measured frequencies. In addition, the equivalent circuit model could be used to quantify the values of the circuit elements to compare the specific system properties (FIGS. 18A-18D).

### 2.4 Cancer Biomarkers

Biomarkers are significant tools that assist in detecting cancer in the early stages. Biomarkers are biological molecules that indicate the condition and the state of the diseases, and they are found in cancerous cells, blood, urine, and other biofluids. These molecules could be DNA, RNA, proteins, and enzymes. Biomarkers are not only important for cancer detection in the early stages but also for monitoring the effect of treatment in cancers, such as chemotherapy or radiation, because the alternation in the expression of the specific protein can be indicated by biomarkers that are measured in the biofluids. There are many cancer biomarkers which can detect different kind of cancers.

### 2.4.1 Breast Cancer Biomarker

Protein biomarkers are widely used to detect breast cancers because they are prognostic markers. DNA markers are also used to detect breast cancers; they give information about the development of tumor growth, but they can't be detected in the early stage of the cancers.

The most common proteomic biomarker for breast cancer is a human epidermal growth factor receptor 2 (Her-2) which is a member of the EGFR family because it is a prognostic marker that can be detected in the breast cancer at the early stage. Her-2 promotes breast cancer growth. The normal levels of Her-2 in the blood are 2-15 ng/ml, but in breast cancer, the levels might be increased to 15-75 ng/ml. The Her-2 levels are considered to diagnose cancer stage and tumor size.

There is an additional proteomic biomarker for advanced breast cancer which is CA 15-3. It is a member of the mucins family. It is mostly used to monitor the therapy of breast cancer. The normal concentration levels of CA 15-3 in the serum are less than 30 U/ml, but in breast cancer, the level will increase in the serum from stage 1 to stage 4 of cancer.

Furthermore, BRCA1 is a DNA biomarker. BRCA1 can be destroyed by mutation that leads to breast cancer. There are other markers which are related to breast cancer such as BRCA2, CAE and CA 27.29.

### 2.4.2 Extracellular Matrix Protein 1 (ECM-1)

Extracellular matrix protein 1 is a secreted glycosylated protein, and the gene of ECM1 is mapped to chromosome 1q21 near the epidermal differentiation complex site. The molecular weight of ECM1 is 85 KDa. ECM1 is expressed in different tissues, and it has many functions such as the maintenance of the extracellular matrix, cell proliferation, angiogenesis, and the regulation of T cells migration. ECM1 promotes tumor development and proliferation, also its expression is correlated with metastatic and poor prognosis in cancer.

In addition, ECM1 are carried by the exosomes from the primary tumor to the peripheral blood. It has been reported that high expression of exosomal ECM1 is associated with Stage I-III Colon Cancer. The primary tumor (colon cancer) releases the exosomes carrying ECM1. Then, the exosomes carrying ECM1 will travel through mesenteric vein to the target organ, and they reach to peripheral veins. Finally, exosomal ECM1 will be retained in the target organ. Thus, the circulating biomarkers of cancers are performed in blood obtained from the peripheral vein. The exosomes are small vesicles (50-200 nm). The exosomes are secreted by many types of cells, and they are captured by receptor cells in target organs. The exosomes are biologically active vesicles that present in all cell types, circulation and body fluids. They are enriched in proteins. Exosomes and their cargo (proteins) are the promising biomarkers for cancers. Furthermore, ECM1 has been detected in the circulation of the exosomes of 82% of the breast cancer. Hence, ECM1 present in the circulating exosomes for the breast cancer. However, the exact role of ECM1 in breast cancer formation remain unclear.

In addition, many studies indicate that ECM1 is a breast cancer biomarker, and the level of ECM1 is overexpressed in breast cancer. It has been found that ECM1 is a significant prognostic marker for breast cancer. It has been shown that up to 83% of breast carcinomas overexpress ECM1. In addition, ECM1 is a urine biomarker that can be found in the breast cancer patients urine. It has been reported that ECM1 is one of the urine markers for breast cancer detection.

### Development of Biosensor

### 3.1 Materials

Gold screen-printed Electrodes were purchased from DropSens (USA); Extracellular matrix protein 1 (hECM1) (Conc 50 μg/500 μl) was purchased from R & D system; Anti-hECM-1 (affinity purified sheep IgG) was purchased from R & D system (USA); Superblock (TBS) blocking buffer was purchased from Thermo Scientific (USA); Dithiobis succinimidyl propionate (DSP) was purchased from Thermo Scientific (USA); 2-Propanol was purchased from Fisher Chemical (USA); Phosphate buffered saline (PBS, pH 7.4) was purchased from Gibco (USA); Dimethyl Sulfoxide (DMSO) was purchased from Fisher Chemical (USA); electrochemical impedance device was from Gamry (USA), and distilled water was used.

### 3.2 Methods

### 3.2.1 Preparing the Artificial Urine

Organic components: 6 g/L of urea, 0.5 g/L of creatinine.

Inorganic components: 2 g/L of potassium chloride, 0.5 g/L of magnesium chloride, 0.25 g/L of calcium chloride, 2 g/L of sodium sulfate, 0.85 g/L of ammonium phosphate, 0.85 g/L of ammonium diphosphate. Adjust the pH to 7.

### 3.2.2 Stage 1

### Modification of the Electrochemical Biosensor

Flush electrodes with isopropyl alcohol followed by PBS. Then, do Vacuum desiccation. Treat the sensor with 10 mM DSP dissolved in DMSO for 1 hour to immobilize the antibody. Incubate the sensor with the capture antibody solution (Anti-hECM1: 1 μg/ml) for 30 min. Treat the sensor with superblock blocking buffer solution for 30 minutes. Aspirate super blocking buffer.

### Experiment Conditions

After modifying the sensor, the chip is injected with plain urine (control), and impedance measurement is obtained. However, for the urine with ECM1 protein, after aspirating super blocking buffer step, the urine including ECM1 protein is injected into the chip for 15 min. Then, the impedance measurement is obtained. 10, 50, 100 pg/ml of ECM1 were tested in the urine. A different chip was used for each concentration.

### Electrochemical Measurement

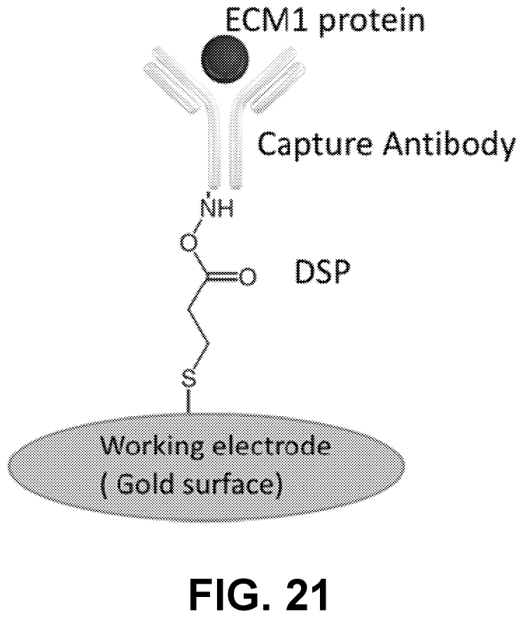

Electrochemical measurements were performed with potentiostat Advanced Electrochemical System Gamry Analyst. All experiments were carried out with a three-electrode system that is comprised of a platinum reference electrode, a counter carbon electrode and gold working electrode (4 mm). Electrochemical impedance spectroscopy is used to characterize the affinity between the antibody and the antigen on the electrode surface that could be screened as the impedance changes. The ECM1 protein is detected with the capture antibody. FIG. 21 shows an example concept of the affinity between the antibody and the antigen in accordance with various aspects of the present disclosure. The electrochemical tests include an open circuit potential (OCP) and Electrochemical impedance spectroscopy (EIS) at Eoc with an amplitude of 10 mV and frequency of range of 10,000 Hz to 0.01 Hz. The change in impedance (ΔZ) is analyzed at different frequencies to obtain the optimum frequency for breast cancer detection. Modified Randel's circuit is used to fit the impedance spectra and identify the electrochemical nature of binding leading to the classification of specific versus nonspecific interactions on the microelectrodes, and to test % change in capacitance.

### 3.2.3 Stage 2

### Modification of the Electrochemical Biosensor

Clean the chip with isopropyl alcohol followed by PBS. Then incubate the chip (only the gold surface-working electrode) with 10 μl urine for 15 min at room temperature to make the chip ready for immunoassay. Then aspirate the urine and Inject 10 μl DMSO into the chip for 15 min at room temperature. DMSO is used as the solvent for the biochemical linker for immobilization of the antibody for the small molecule of ECM1. Then, aspirate DMSO and treat the Chip with 10 mM DSP dissolved in DMSO for 30 min at room temperature. DSP is used to immobilize the capture Antibody (hECM1) on the sensing electrodes. DSP contains two disulfide functional ends that bind to the gold electrode and ester that bind with a capture antibody. Then aspirate the DSP and wash the chip one time with DMSO. Then incubate the chip with 10 μl capture antibody solution (Anti-hECM1: 2 μg/ml) for 15 min at room temperature (the dilution of the antibody was prepared from stock). Then, aspirate the capture antibody and wash the chip with PBS 3×. Then, Treat the chip with 10 μl Super blocking buffer (TBS) solution for 15 minutes at room temperature. Super blocking buffer (TBS) is used to block nonspecific binding. Then, aspirate the Super blocking buffer and wash the chip with distilled water 3×

### Experiment Conditions

After modifying the sensor, the chip is injected with plain urine (control), and impedance measurement is obtained. However, for the urine with ECM1 protein, after aspirating super blocking buffer step, the urine including ECM1 protein is injected into the chip for 15 min. Then, the impedance measurement is obtained. In this stage, two sets are tested. First set at EOC, 10, 50, 100 pg/ml ECM1 in the urine were tested. The second set at Eref, 10, 50, 200 pg/ml ECM1 in the urine were tested. Each concentration was tested in different chip.

### Electrochemical Measurement

The same protocol from stage 1 was followed. However, in this stage, electrochemical impedance spectroscopy (EIS) was used at Eoc with an amplitude of 10 mV and frequency of range of 10,000 Hz to 0.01 Hz. Also, electrochemical impedance spectroscopy (EIS) was used at Eref with an amplitude of 0.2 mV and frequency of range of 10,000 Hz to 0.01 Hz. The change in impedance (ΔZ) is analyzed at different frequencies to obtain the optimum frequency for breast cancer detection. Modified Randel's circuit is used to fit the impedance spectra and identify the electrochemical nature of binding leading to the classification of specific versus nonspecific interactions on the microelectrodes, and to test % change in capacitance.

### 3.2.4 Stage 3

### Antibody Saturation

Clean the sensor with 70% alcohol followed by the distilled water. The working electrode of the sensor is coated with DSP (only working electrode) for 30 minutes in the room temperature, then unbounded DSP was aspirated. After that, add PBS on the sensor as a control and incubate it on the sensor for 30 minutes, then EIS is obtained. After EIS, incubate the sensor with 0.5 μg/ml hECM1 antibody for 30 minutes at room temperature, then read out the result by EIS. Then, incubate the same sensor with additional 0.5 μg/ml (cumulative 1 ug/ml) hECM1 antibody for 30 minutes at room temperature, then read out the result by EIS. Repeat the same step for the rest of the cumulative concentrations which are 2, 4, 5, 7, 10, 15 μg/ml. From EIS data, CPE modal is used to test the change in capacitance. One chip was used for all concentrations.

### Electrochemical Measurement

The electrochemical tests include an open circuit potential (OCP) for 600 seconds, Electrochemical impedance spectroscopy (EIS) at Eoc with an amplitude of 10 mV and frequency of range of 10,000 Hz to 0.01 Hz, and an open circuit potential (OCP) for 100 seconds. From EIS data, the change in impedance (ΔZ) is analyzed at 0.0125 Hz, and CPE circuit is used to test the % change in capacitance.

### 3.2.5 Stage 4

### Modification of the Electrochemical Biosensor

Cleaned the sensor with 70% alcohol followed by the distilled water. The working electrode of the sensor is coated with DSP (only working electrode) for 30 minutes in the room temperature, then unbounded DSP was aspirated. Incubate the sensor with 40 μl of hECM1 antibody 7 μg/ml (all electrodes) for 30 minutes at room temperature. Aspirate the AB and wash the sensor 1× with PBS. Incubate the sensor with 40 μl of 5% BSA for 30 minutes at room temperature. Aspirate the BSA and wash the sensor 1× with PBS.

### Experiment Conditions

After BSA step, the sensor was incubated with urine (control) for 15 minutes, then EIS is obtained. Then, the urine including ECM1 protein is injected into the chip for 15 min. Then, the EIS is obtained. 1, 5, 10, 20, 50 100 pg/ml ECM1 were tested. Incubate each concentration for 15 minutes, then EIS is run. Each concentration was tested in the different sensor, and in each sensor, EIS is run two times one for the control and the other for the test with ECM1 concentration. Hence, each concentration of ECM1 has its control.

### Electrochemical Measurement

The electrochemical tests include an open circuit potential (OCP) for 600 seconds, Electrochemical impedance spectroscopy (EIS) at Eoc with an amplitude of 10 mV and frequency of range of 10,000 Hz to 0.01 Hz, and an open circuit potential (OCP) for 100 seconds. From EIS data, the change in impedance (ΔZ) is analyzed at 0.0125 Hz, and CPE circuit is used to test the % change in capacitance.

### 3.2.6 Stage 5

### Modification of the Electrochemical Biosensor

Clean the sensor with 70% alcohol followed by the distilled water. The working electrode of the sensor is coated with DSP (only working electrode) for 30 minutes in the room temperature, then unbounded DSP was aspirated. Incubate the sensor with 40 μl of hECM1 antibody 7 μg/ml (all electrodes) for 30 minutes at room temperature. Aspirate the AB and wash the sensor 1× with PBS. Incubate the sensor with 40 μl of 5% BSA (blocking) for 30 minutes at room temperature. Aspirate the BSA and wash the sensor 1× with PBS.

### Experiment Conditions

After blocking, the sensor was incubated with urine (control) for 15 minutes, then EIS is obtained. Then, the urine including ECM1 protein is injected into the chip for 15 min. Then, the EIS is obtained. 10 pg/ml, 100 pg/ml, 500 pg/ml ECM1 were tested. Incubate each concentration for 15 minutes, then EIS is obtained. One sensor was used for all concentrations, and synthetic urine alone used in the first step will be considered as control.

### Electrochemical Measurement

The electrochemical tests include an open circuit potential (OCP) for 600 seconds, Electrochemical impedance spectroscopy (EIS) at Eoc with an amplitude of 10 mV and frequency of range of 10,000 Hz to 0.01 Hz, and an open circuit potential (OCP) for 100 seconds. From EIS data, the change in impedance (ΔZ) is analyzed at 0.0125 Hz, and CPE circuit is used to test the % change in capacitance.

### 3.2.6 Confocal

Coat the sensor (gold area) with 10 μl DSP for 30 min at 37° C. Coat the gold area with 10 μl mouse anti-human ECM1 for 30 min at 37° C. Then, wash one time with PBS. Coat the gold area with 10 μl ECM1 protein for 30 min at 37° C. Coat the gold area with 10 μl sheep anti-human ECM1 for 30 min at 37° C. Then wash one time with PBS. Coat the gold area with 10 μl rabbit anti-sheep IgG FITC for 30 min at 37° C. The biosensor is mounted with fluorogel, then the images are taken

### 3.2.7 Statistical Analysis

Statistical analysis using student's t-test was performed. The data are represented as mean±standard deviation (SD) of three independent experiments in triplicate. P<0.05, is considered as significant. Student's t-test was performed to compare the change in capacitance and impedance obtained between different concentrations of ECM-1protein.

### 3.3 Results

### 3.3.1 Stage 1

### (A) Bode Plot

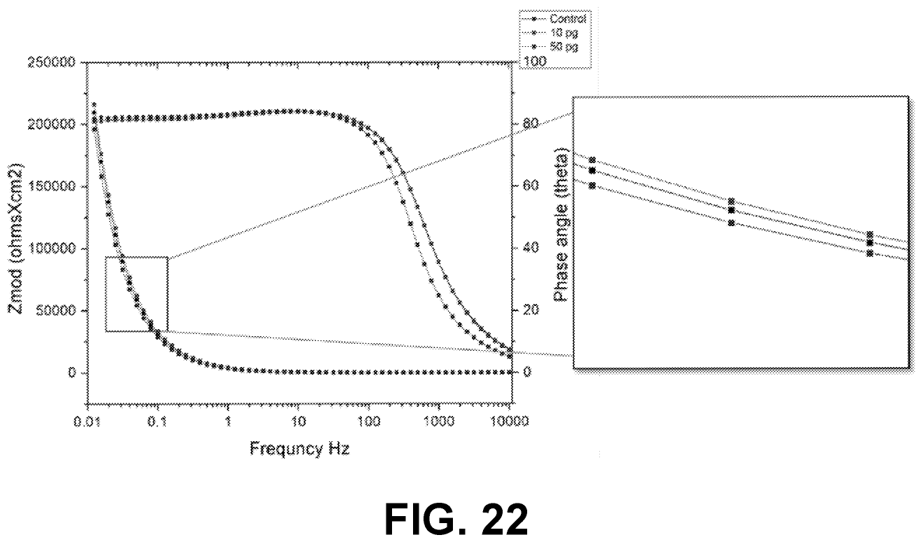

Bode plot includes frequency on the X-axis and 2 Y axes, one for impedance and one for phase angle. Bode plot is very important and useful tool to identify the optimum frequency to detect the ECM1 biomarker. The change in impedance (ΔZ), between the control and test samples, is calculated. Thus, the frequency with the highest ΔZ will be the optimum frequency that can detect the ECM1 biomarker. The result shows that (FIG. 22) the order of the impedance is as follows 10 pg/ml>Control>50 pg/ml. The results of impedance for ECM1 concentrations are not consistent.

### (B) Change in Impedance

From bode plot, different frequencies were tested (24.9, 7.8, 0.0125 Hz) to know what the best frequency is to detect ECM1. The optimum frequency should have the highest change in impedance between the control and the tests (ΔZ). It can be seen that (Table 2), ΔZ for 10 pg/ml ECM1 is higher than 50 pg/ml ECM1 in all frequencies because the data are not in order, so ΔZ 50 pg/ml should be higher than 10 pg/ml. However, in ΔZ of 10 pg/ml ECM1, 0.01259 Hz is the best and optimal frequency compared with 7.8 Hz and 24.9 because it has the highest change of impedance between the control and the test.

TABLE 2
Different Frequencies for the Changing of the Impedance in
the Control and ECM1 Test. 24.9 Hz, B 7.8 Hz, 0.0125 Hz

| | | Name | Zmod | ΔZ |
|---|---|---|---|---|
| 0.01259 | Hz | Control | 1668961 | 0 |
| | | 10 pg | 1720963 | 52002 |
| | | 50 pg | 1561944 | −107017 |
| 7.8 | Hz | Control | 4460.49 | 0 |
| | | 10 pg | 4760.882 | 300.392 |
| | | 50 pg | 3991.926 | −468.564 |
| 24.9 | Hz | Control | 1499.131 | 0 |

| | | |
|---|---|---|
| 10 pg | 1599.115 | 99.984 |
| 50 pg | 1335.79 | −163.341 |

### 3.3.2 Stage 2

### (1) An Electrochemical Test Result at EOC

### (A) Bode Plot

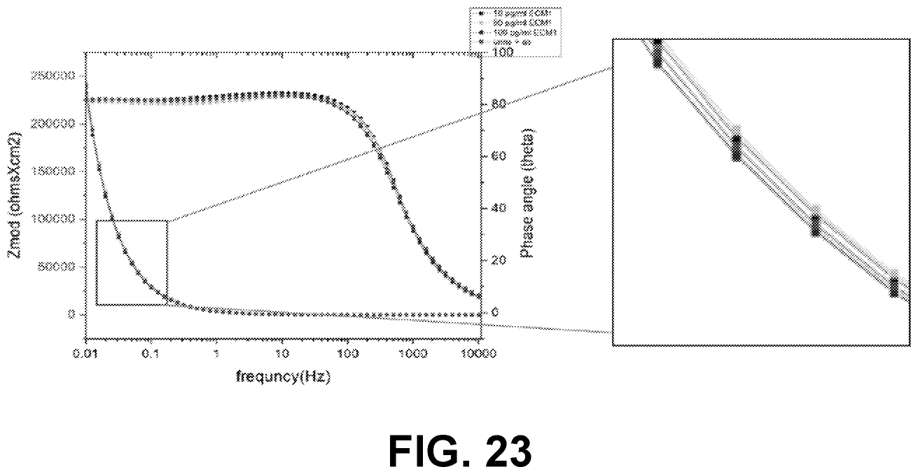

The result shows that (FIG. 23) the order of the impedance is as follows 50 pg/ml>10 pg/ml>Control>100 pg/ml. The results of impedance for ECM1 concentrations are not consistent, also there is no decreasing or increasing order of ΔZ as increasing the concentrations of ECM1 protein.

### (B) Change in Impedance

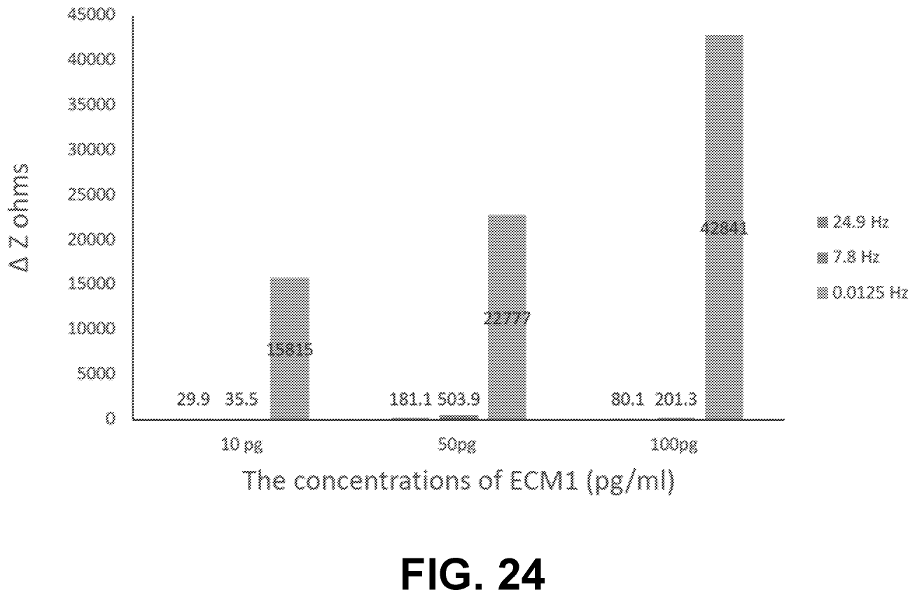

From bode plot, different frequencies were tested (24.9, 7.8, 0.0125 Hz) to see what the best frequency is to detect ECM1 that has the highest change in impedance between the control and the tests (ΔZ). The result shows that (FIG. 24) the maximum change in impedance is identified at 0.0125 Hz, when compared with the control, indicating the presence of 100, 50 and 10 pg/ml EMC1 protein in the urine solution in Eoc. ΔZ increases from 10 pg/ml to 100 pg/ml ECM1.

### (C) Change in Capacitance

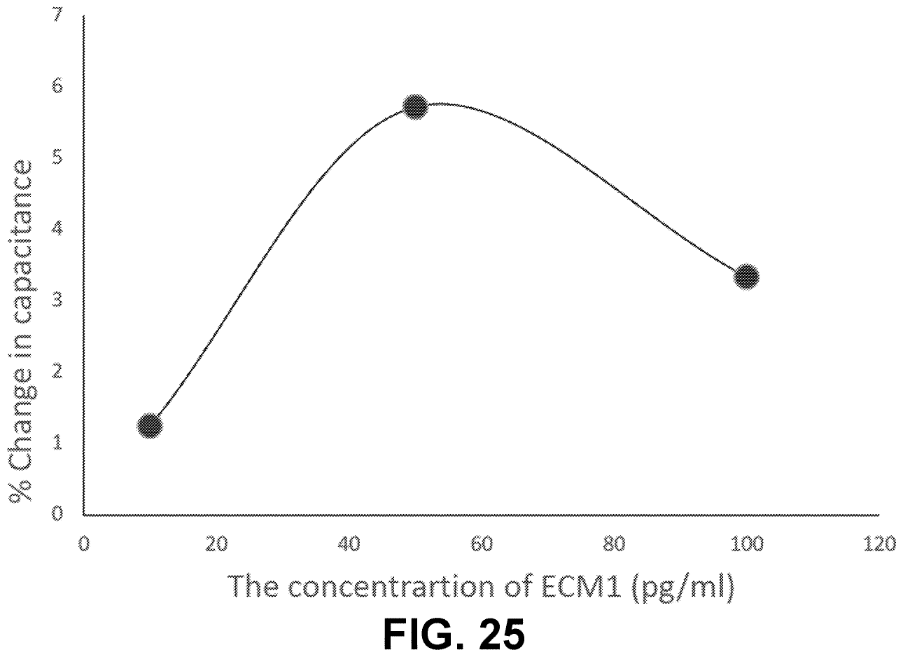

From EIS data, modified Randle's circuit is used to fit the impedance spectra and identify the electrochemical nature of binding leading to the classification of specific versus nonspecific interactions on the microelectrodes. From Randle's circuit, the capacitance was obtained and then change in capacitance from the control urine samples were calculated. The result shows that (FIG. 25) the % change of the capacitance get increased for 10 and 50 pg/ml, however, it gets decreased to 100 pg/ml. Thus, the capacitance change is not consistent because it should also increase for 100 pg/ml.

### (2) An Electrochemical Test Result at Eref

### (A) Bode Plot

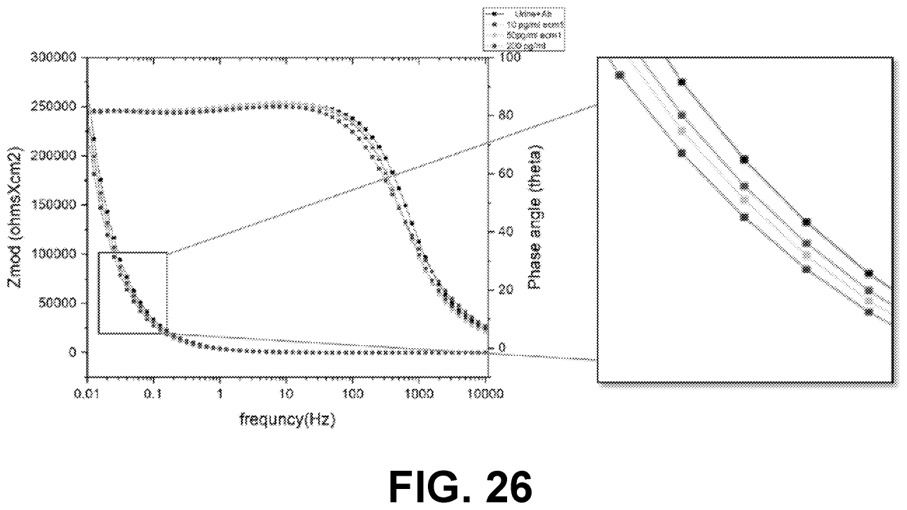

The result shows that the order of the impedance is as follows Control>200 pg/ml 50 pg/ml>10 pg/ml ECM1. The concentration of ECM1 are in the order of increasing, so they started from 10 pg/ml to 200 pg/ml (FIG. 26). The control is supposed to have the lowest impedance because it is the less resistance, but here the control has the highest impedance, which is not acceptable. In addition there is not much difference in impedance between the ECM1 concentration.

### (B) Change in Impedance

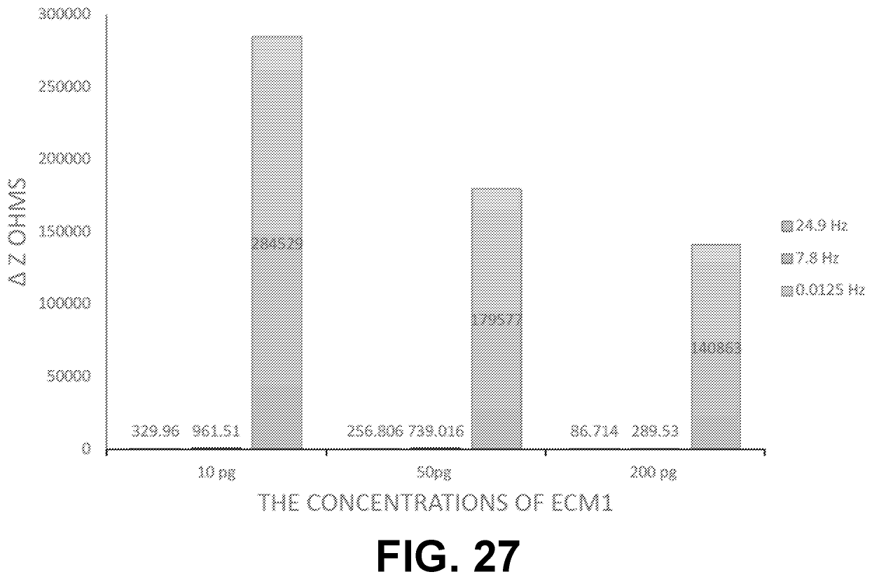

From bode plot, different frequencies are tested (24.9, 7.8, 0.0125 Hz) to know what the best frequency is to detect ECM1 that has the highest change in impedance between the control and the tests (ΔZ). The result shows that (FIG. 27) the maximum change in impedance is identified at 0.0125 Hz, when compared with the control, indicating the presence of 200, 50 and 10 pg/ml EMC-1 protein in the urine solution in Eref. However, the ΔZ decreases from 10 pg/ml to 200 pg/ml ECM1.

### (C) Change in Capacitance

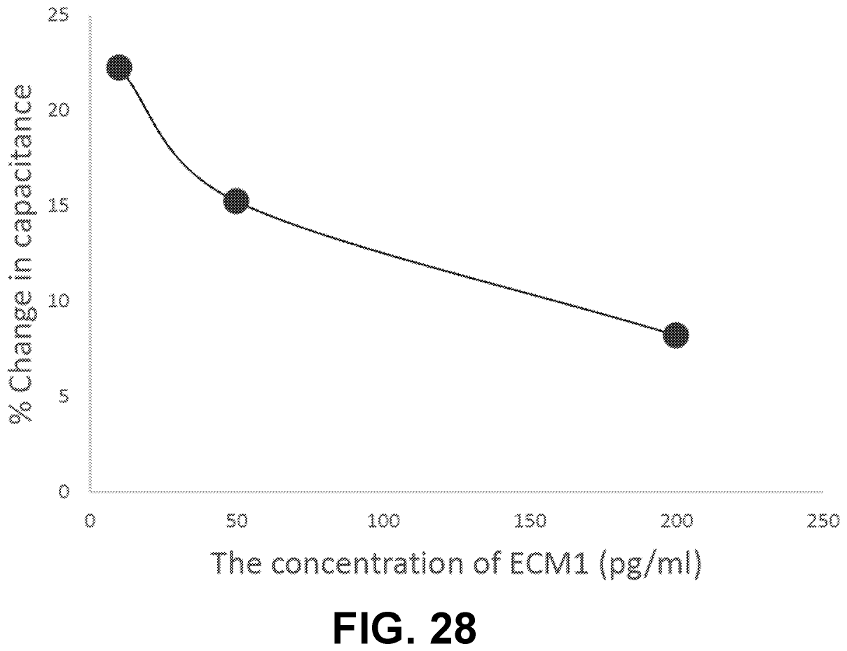

From Randle's circuit, the change in capacitance was tested. The result shows that (FIG. 28) the % change of the capacitance gets decreased from 10 to 200 pg/ml. The change in capacitance should increase from the lower concentration of the ECM1 to the higher concentration of the ECM1. However, in this result, the change in the capacitance decreases from the lower concentration of the ECM1 to the highest concentration of the ECM1, and again not acceptable according to the principles of electrochemistry.

### 3.3.3 Stage 3 (Antibody Saturation)

### (A) Bode Plot

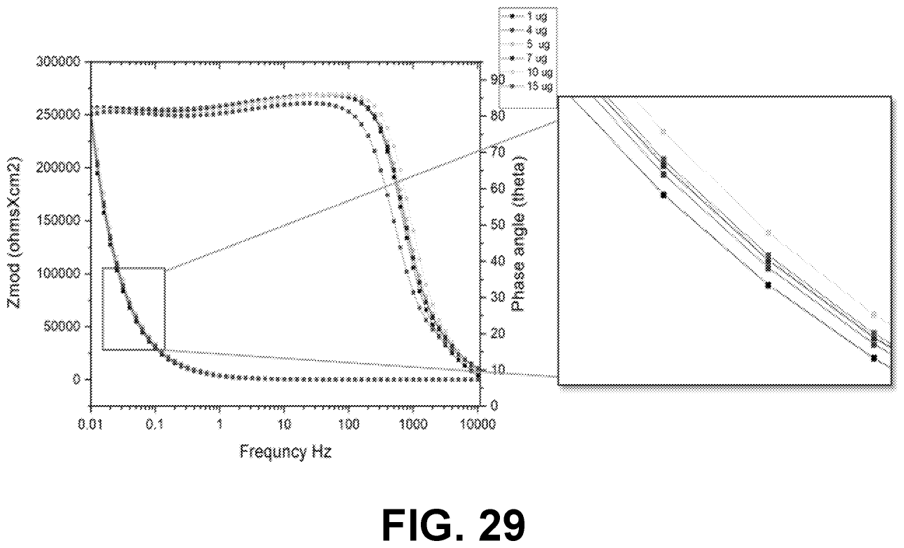

The result shows that the order of the impedance is as follows 10 μg/ml>15 μg/ml>7 μg/ml>5 μg/ml>4 μg/ml>1 μg/ml hECM1 antibody (FIG. 29). The impedance of the antibody concentrations increases from 1 μg/ml to 10 μg/ml, then when the concentration reached 15 μg/ml the impedance gets a decrease. That means the antibody get saturated on the sensor when it reached to 10 μg/ml. Hence, 10 μg/ml is the optimal concentration of the antibody on the sensor.

### (B) Change in Impedance

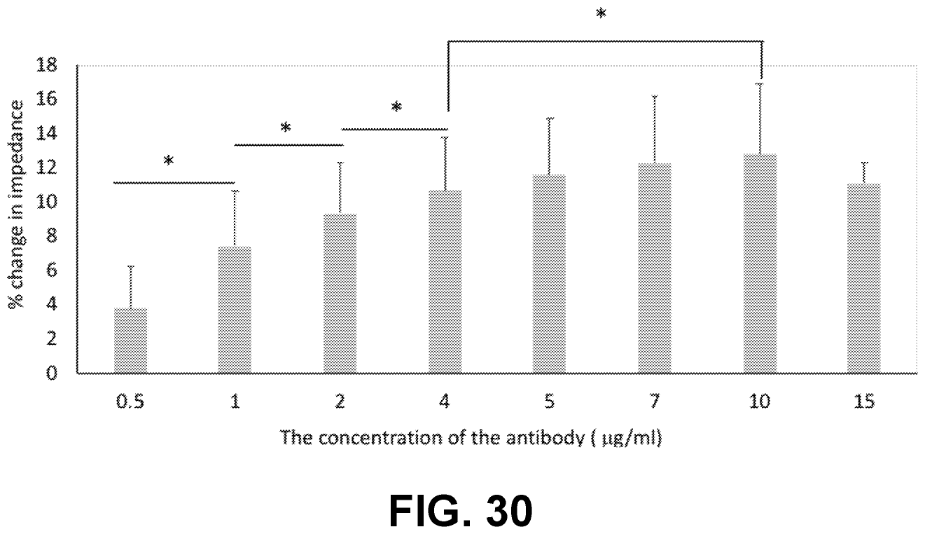

From bode plot, % change in impedance was tested at 0.0125 Hz. The result shows that (FIG. 30) The % change in impedance of the antibody concentrations increases from 0.5 μg/ml to 10 μg/ml, then when the concentration reached 15 μg/ml the % change in impedance decreases. From this result, it was observed that after 7 μg/ml concentration of antibody the sensor is saturated and there is a decline in change in impedance at 15 μg/ml.

### (C) Change in Capacitance

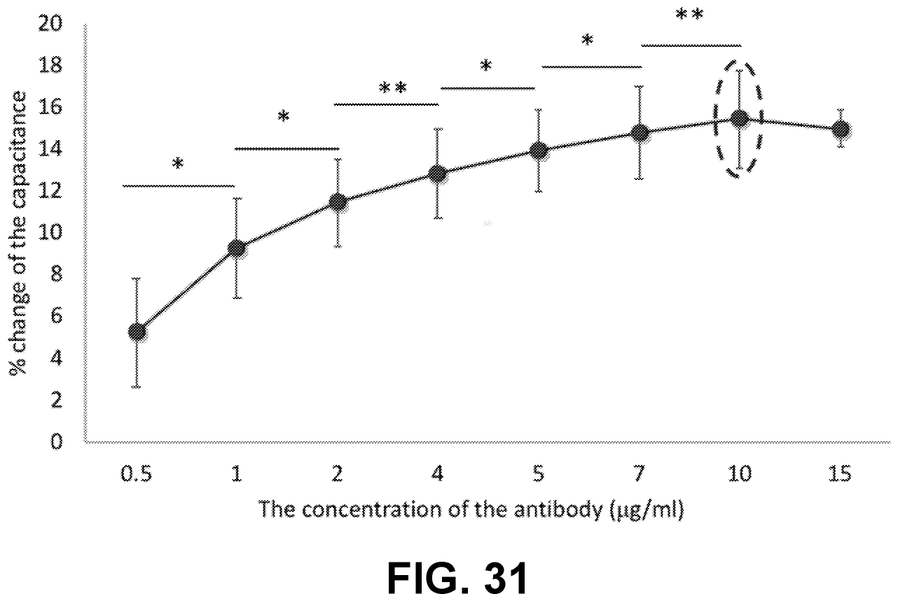

From the CPE circuit, the change in capacitance is tested at 0.0125 Hz. Cnsistent with change in impedance data, the % change in the capacitance increases from 0.5 μg/ml to 10 μg/ml, then it decreases for 15 μg/ml (FIG. 31). As a result, 10 μg/ml is the optimal concentration of the antibody concentration because the antibody is saturated. There is significant increase in change in capacitance between each concentration studied.

### 3.3.4 Stage 4

### (A) Bode Plot

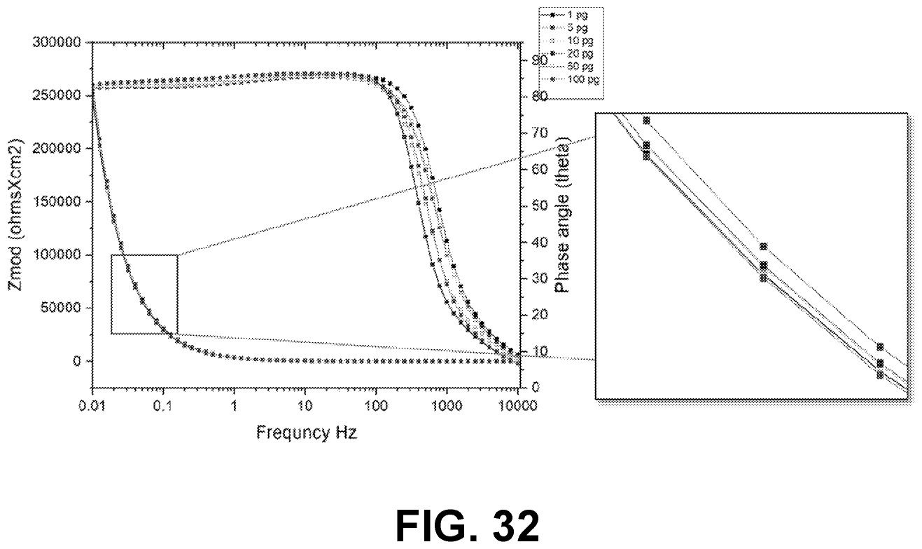

As can be seen from the result, the order of the impedance is as follows 5 pg/ml>20 pg/ml>10 pg/ml>50 pg/ml>1 pg/ml>100 pg/ml ECM1 (FIG. 32). The results of impedance for ECM1 concentrations are not consistent at all.

### (B) Change in Impedance

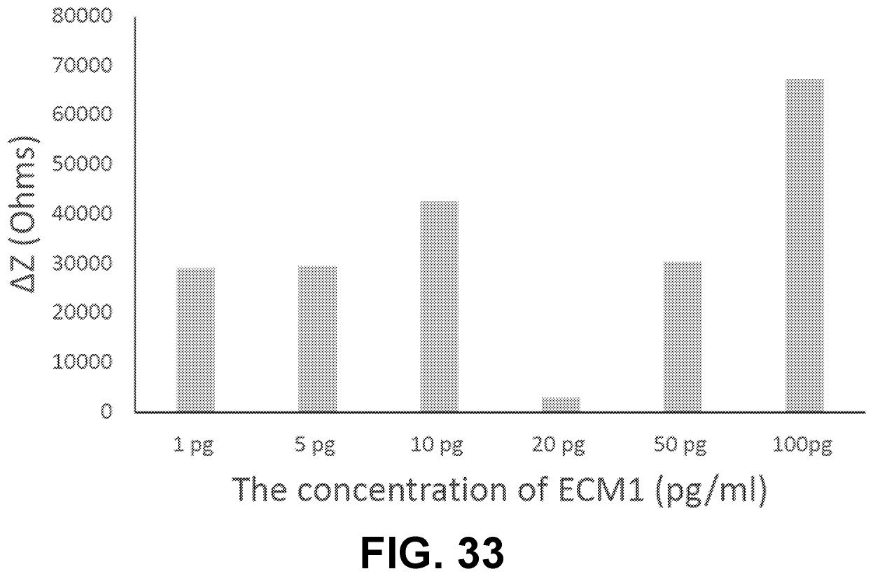

From bode plot, the change in impedance was tested at 0.0125 Hz. The result shows that (FIG. 33) the change in impedance of the ECM1 concentrations increases from 1 pg/ml to 10 pg/ml, then it decreases for 20 pg/ml. At 50 pg/ml the impedance increases slightly again. Finally, the impedance increases at 100 pg/ml. Thus, the date is not consistent.

### (C) Change in Capacitance

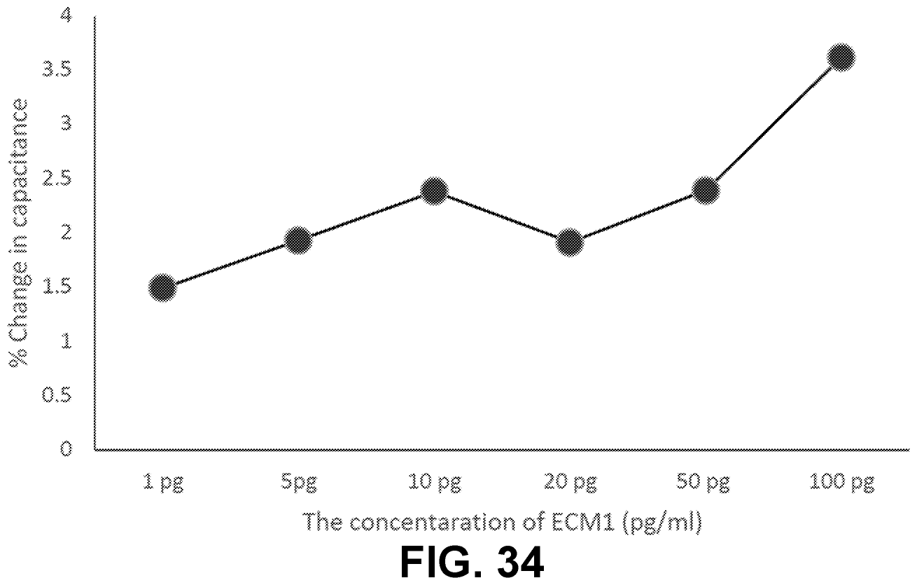

From the CPE circuit, the change in capacitance is tested at 0.0125 Hz. The result shows that (FIG. 34) the % change in the capacitance increases from 1 pg/ml to 10 pg/ml, then it decreases for 20 pg/ml. From 20 pg/ml to 50 pg/ml the % change in capacitance increases. The data are not in order.

### (D) The Characterization of the Sensor

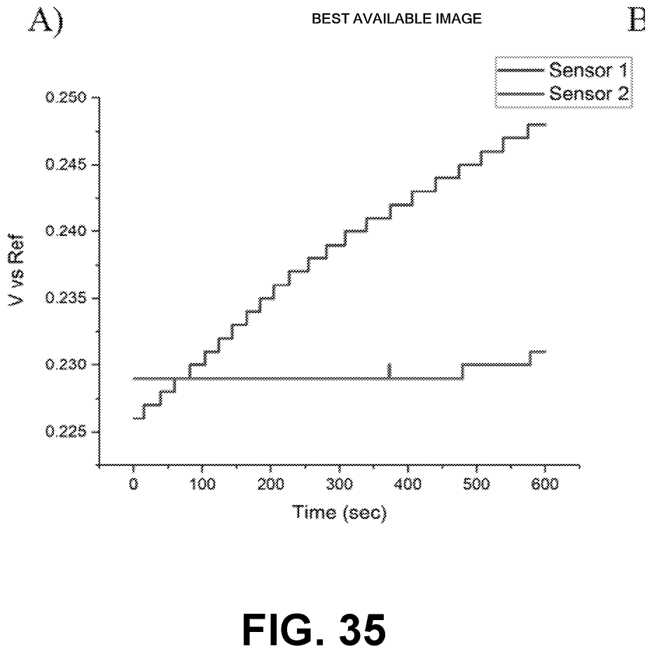

Open circuit potential is used to measure the open circuit voltage (Eoc) of an electrochemical cell. OCP graph indicates the stability of the experiment as well as the initial status of the sensor. OCP is the voltage at Eoc versus the time. The characterization of the sensor was tested by doing OCP and SEM. Two OCP for two different sensors were tested. The result shows that the OCPs varies between the two sensors tested from 230 mV to 250 mV. This difference because of the defect on the surface of the sensor. It can clearly be seen that the defect on the sensor by SEM (FIG. 35).

### 3.3.5 Stage 5

### (A) Bode Plot

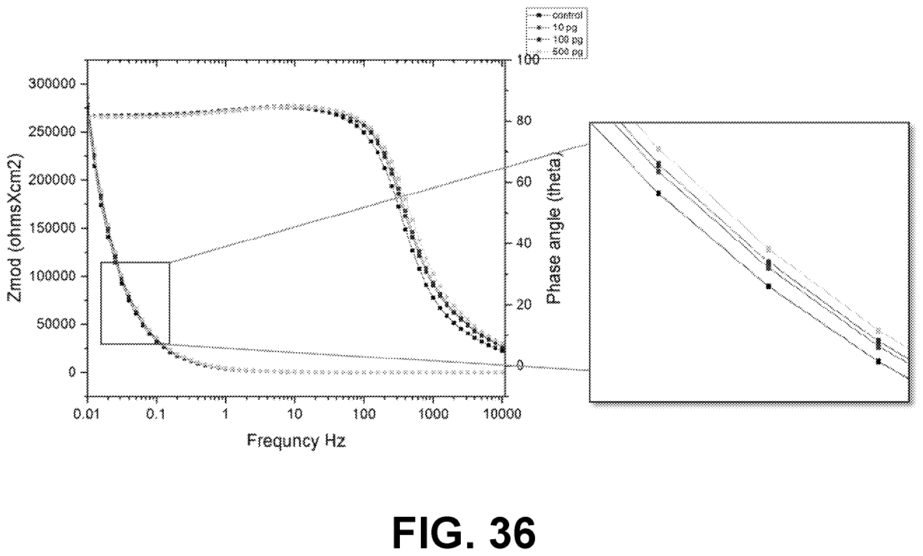

The result shows that the order of the impedance is as follows control >10 pg/ml>100 pg/ml>500 pg/ml ECM1 a (FIG. 36). The impedance of ECM1 concentrations are in the order of increasing. The bode plot shows that the data is very consistent.

### (B) Change in Impedance

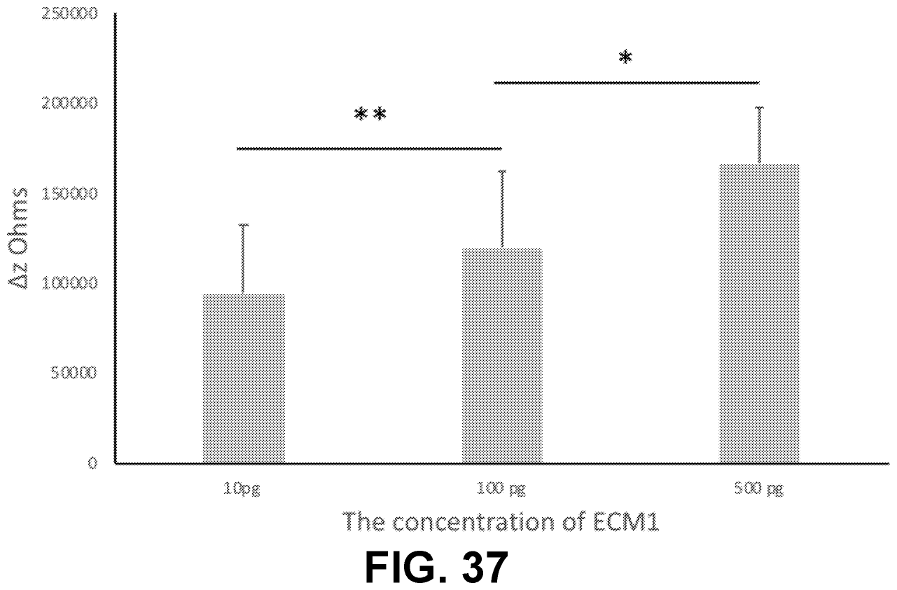

From the bode plot, the change in impedance is tested at 0.0125 Hz. The result shows that (FIG. 37) the change in the impedance increases from 10 pg/ml to 500 pg/ml. Az for 10 pg/ml is around 100000 ohms, Δz for 100 pg/ml is around 130000 ohms, and Δz for 500 pg/ml is around 180000 ohms. It can be seen that there is a difference between Δz's for the ECM1 concentrations, and the data is very consistent.

### (C) Change in Capacitance

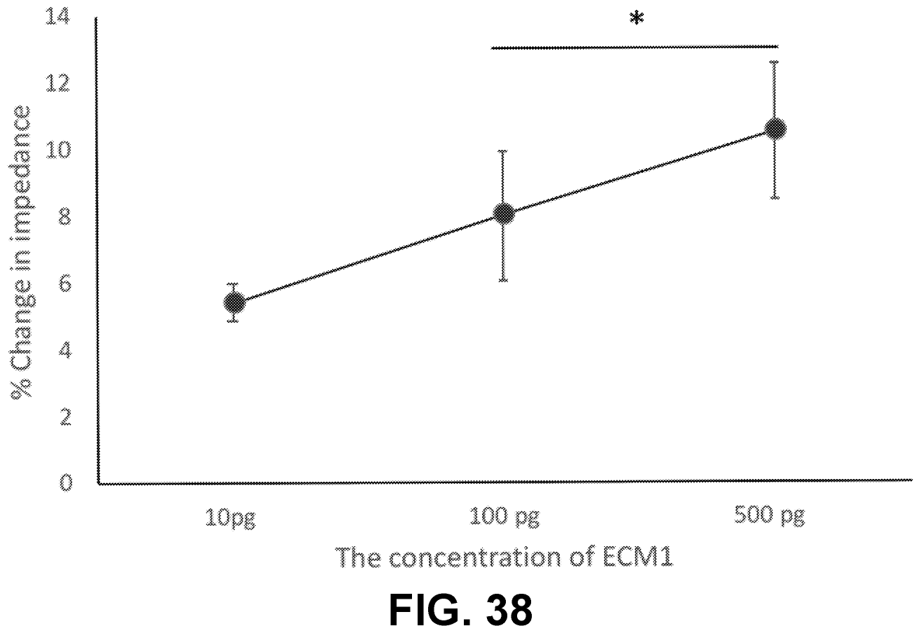

Using the CPE equivalent circuit the capacitance was obtained and the change in capacitance from the control was evaluated at 0.0125 Hz. The result shows that (FIG. 38) the % change in the capacitance increases from 10 pg/ml to 500 pg/ml, from 5% to 11%. As a result, the data is consistent because % change in the capacitance is increased from the lower to the higher concentration of ECM1.

### 3.3.6 Confocal

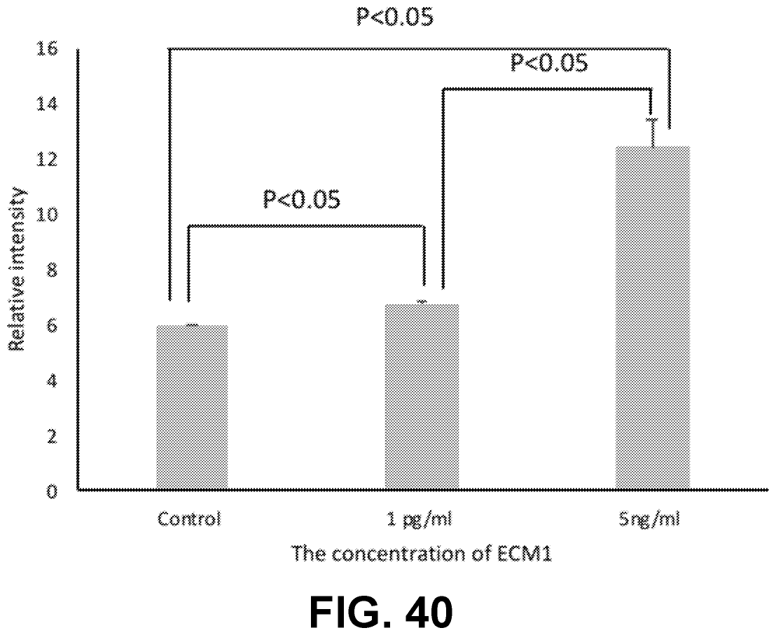

The confocal was performed to confirm the immobilization of the antibody on the sensor. The result shows that the antibody was immobilized on the sensor successfully. Moreover, it can be seen that the amount of the protein is very less in the 1 pg/ml ECM1 on the sensor compared with 5 ng/ml ECM1on the sensor. Image J software was used to quantify the intensity of green fluorescence. The results are shown in FIG. 40. It was observed that there is a significant increase in the fluorescent intensity in the case of 5 ng/ml compared to 1 pg/ml concentration of ECM-1. Results clearly demonstrated the affinity of the antigen-antibody on the sensor.

### 3.4 Discussion

According to the WHO breast cancer is the second cause of death among all kinds of cancer, accounting for 571,000 death annually. Early diagnosis is very important for breast cancer because that leads to successful treatment and decrease the rate of death. For this aim, there are many current methods to detect breast cancer in the early stages. However, most of them are expensive, painful, time-consuming and less sensitive. Because of this limitation, developing a new tool which is sensitive, rapid, economic is desired. The biosensor is a bioanalytical device that is used to detect analytes by converting them into an electrical signal to be analyzed in an electronic form. The biosensor includes two components which are Receptor recognition elements and traducers. Receptor recognition elements such as nucleic acid, protein, enzyme, antibody or antigen that attract the analytes and convert them into electrical energy. The transducer is the device which is used to transform the electrical energy to a signal such as electrochemical then the signal will be analyzed in electronic form. Moreover, Biomarkers are a very important aspect in the biosensor. Biomarkers are significant tools that assist in detecting cancer in the early stages. They are found in cancers cells, blood, urine, and other biofluids. These molecules could be DNA, RNA, proteins, and enzymes. Biomarkers also play a crucial role in monitoring the effect of the treatments in cancers such as chemotherapy or radiation.

Several studies have been done using different types of the biosensor to detect different kinds of breast cancer biomarkers. All these studies reported successful results, and the biosensors can detect the breast cancer with small amounts of biomarkers. However, there are many limitations in reported biosensors. Hence their wide range of applications in cancer detection is questionable because most of these biosensors used DNA, cellular and serum biomarkers for breast cancer detection. That makes the methods costly, reliant on technical skills, and not patient driven tools. To overcome these limitations, various aspects of the present disclosure develop a biosensor to detect breast cancer biomarker in urine. Urine biomarker was chosen because urine is non-invasive, and it makes the biosensor economic and easy to use. It has been found that ECM1 and MAST 4 are potential urine biomarkers for breast cancer. Accordingly, in this Example, the electrochemical biosensor is used to detect ECM1 in the urine. This type of biosensor was chosen because it is inexpensive, rapid, easy to use and simple. Also, it is classified as a point-of-care device. Electrochemical impedance spectroscopy (EIS) is used to obtain the readout. EIS is an electrochemical technique that is used to characterize the binding of the different entities on the electrode surface, which can be screened as the impedance changes. Electrical impedance is the measure of the resistance when AC voltage is applied.

After choosing the biomarker and biosensor, the optimized protocol was developed for the electrochemical biosensor to immobilize the antibody which binds to the antigen on the biosensor, which is aim 1. Then, the data was analyzed by using a bode plot which includes frequency on the X-axis and 2 Y axes, one for impedance and one for phase angle. Bode blot is very important and useful tool to identify the optimum frequency to detect the ECM1 biomarker. After that, the change in impedance (ΔZ), between the control and test samples was calculated by this equation. ΔZ=impedance of the test impedance of control. Then, the frequency with the highest ΔZ will be the optimum frequency that can detect the ECM1 biomarker. An equivalent circuit was developed using a modified Randle's circuit which indicates the interaction between ECM1 and the sensor platform. In this Example, the capacitance was used as a measure of this electrochemical interaction using the following equation:

$$\% Change in Capacitance = \frac{capacitance of the test - capacitance of control}{capacitance of control} \times 100$$

The protocol development for the biosensor went through 5 stages. In stage 1, as it was mentioned 1 μg/ml of ECM1 antibody was used to coat the sensor, also, when the sample was run in EIS, EOC set was used. After plot the bode plot, different frequencies were tested to know what is the best frequency that gives high change in impedance. However, the result was not consistent.

In stage 2, the protocol is modified to get the data that is expected. Increasing the concentration of ECM1 antibody to 2 μg/ml and two sets of impedance measurements were carried out; one at Eoc and the other at Eref. At Eoc, the impedance will measure at varying potential, but at the Eref, specific potential will be taken which is 0.2V for OCP. After plot the bode plot, different frequencies were tested to know what is the best frequency that gives high change in impedance.

In this stage, from the results, it can be seen that Eoc experiment gives unstable results and the impedance of the concentrations of the ECM1 are not in the order of increasing/decreasing. At Eref, A Z is high between the control and the tests, and the impedance of the concentrations of the ECM1 is in the order of increasing concentrations, and bode plot showed that the order of the impedance is followed as Control>200 pg/ml>50 pg/ml>10 pg/ml. However, Eref is still not sufficient because the impedance of the control was higher than the ECM1 concentrations that made Δz decreased from 10 pg/ml to 200 pg/ml, but in EOC data, Δz increased from 10 pg/ml to 100 pg/ml. from this stage, 0.0125 Hz is the best frequency because Δz was high compared with other frequency in the both EOC and Eref sets. Since consistent data wasn't produced, the process moved to stage 3.

In stage 3, from stage 1 and 2 the antibody concentration is not sufficient, so the attention was turned to the concentration of the antibody. The antibody saturation test was done, to know what the optimal concentration of the antibody on the sensor is when the antibody gets saturated on the sensor. The change in impedance at 0.0125 was tested because it is the optimal frequency. Also, an equivalent circuit was developed using a modified CPE circuit to measure the % change of the capacitance. From the result, 10 pg/ml is the optimal concentration of the antibody on the sensor because the antibody is got saturation in this concentration.

In stage 4, after deciding the sufficient antibody concentration for the protocol, different concentrations of the ECM1 were tested, and different sensors were used for each concentration. However, the result was not efficient, and the data was not consistent because the impedance of the ECM1 concentration is not in order for decreasing or increasing. Further, SEM was carried out to characterize the sensor. From OCP and SEM results, the initial nature of the biosensors is not uniform because of the defect. Hence, each biosensor has different sensitivity and defect.

In stage 5, one sensor was used for all ECM1 concentrations. The results show that the data was very consistent. There was a clear increase in percentage changes in capacitance and in impedance from 10 pg/ml to 500 pg/ml ECM1. There is a linear increase in impedance from 1 pg/ml to 500 pg/ml ECM1 protein. After all these stages, confocal was performed. The Confocal results confirm the immobilization of the Ab on the sensor and the affinity of the antibody and antigen.

### 3.5 Summary

The optimal frequency to detect the ECM1 is 0.0125 Hz, because it has the maximum change in impedance (ΔZ). From antibody saturation test, 10 μg/ml is the optimal concentration of the antibody to completely saturate the working electrode (WE) of the sensor for the effective electrochemical response. In addition, each sensor has a different defect and sensitivity. The biosensor is able to detect ECM1 in the urine with a low amount. The confocal result confirms the immobilization of the antibody on the sensor and the affinity of the antibody and antigen.

### Calibration and Sensor Characterization

### 4.1 Materials

Gold screen-printed Electrodes were purchased from DropSens (USA); Extracellular matrix protein-1 (hECM1) (Conc 50 μg/500 μl) was purchased from R & D system; Anti-hECM-1 (affinity purified sheep IgG) (Conc 25 μg) was purchased from R & D system (USA); Dithiobis succinimidyl propionate (DSP) was purchased from Thermo Scientific (USA); Phosphate buffered saline (PBS, PH 7.4) was purchased from Gibco (USA); electrochemical impedance device was from Gamry (USA), and distilled water was used.

### 4.2 Methods

### 4.2.1 Modification of the Electrochemical Biosensor

Cleaned the sensor with 70% alcohol followed by the distilled water. The working electrode of the sensor is coated with DSP (only working electrode) for 30 minutes in the room temperature, then unbounded DSP was aspirated. Incubate the sensor with 40 μl of hECM1 antibody 7 μg/ml (all electrodes) for 30 minutes at room temperature. Aspirate the AB and wash the sensor 1× with PBS. Incubate the sensor with 40 μl of 5% BSA for 30 minutes at room temperature. Aspirate the BSA and wash the sensor 1× with PBS.

### 4.2.2 Experiment Conditions

Followed by blocking, the sensor was incubated with urine (control) for 15 minutes, then EIS is obtained. Then, the urine including ECM1 protein is injected into the chip for 15 min. Then, the EIS is obtained. 1 pg/ml, 10 pg/ml, 100 pg/ml, 500 pg/ml, 1 ng/ml, 500 ng, ml, 1 pg/ml ECM1 were tested. Incubate each concentration for 15 minutes, then EIS is obtained. The same sensor was used for all concentrations, and all of them have one control. The same steps were repeated with PBS instead of urine to optimize the sensor and to test the efficiency of the antibody and antigen affinity in a different solution.

### 4.2.3 Electrochemical Measurement

The electrochemical tests include an open circuit potential (OCP) for 600 seconds, Electrochemical impedance spectroscopy (EIS) at Eoc with an amplitude of 10 mV and frequency of range of 10,000 Hz to 0.01 Hz, and an open circuit potential (OCP) for 100 seconds. From EIS data, the change in impedance (ΔZ) is analyzed at 0.0125 Hz, and CPE circuit is used to test the % change in capacitance.

### 4.2.4 ELISA

Coat 96 well plate with ECM1 protein (serial dilution), incubate the plate overnight at 4 degrees. Next day, prepare block buffer (5% BSA) and wash buffers (PBS with 0.05% tween). Wash wells with 280 μl PBST 3×. Add 280 μl of blocking buffer, then incubate the plate 2 hours at room temperature. Wash wells with 280 μl PBST 3×. Add 100 μl of the primary antibody (sheep anti-human 1:5000), incubate the plate 1 hour at room temperature. Wash wells with 280 μl PBST 3×. Add 100 μl of secondary antibody (rabbit anti-sheep IgG-HRP 1:10,000), incubate the plate 1 hour at room temperature. Add 100 μl substrate solution (TMB). Incubate the plate until color change. Add 50 μl stop solution (sulfuric acid). Read the plate at 450 nm.

### 4.2.5 Statistical Analysis

Statistical analysis using student's t-test was performed. The data are represented as mean±standard deviation (SD) of three independent experiments in triplicate. P<0.05, is considered as significant. Student's t-test was performed to compare the change in capacitance and impedance obtained between different concentrations of ECM-1protein.

### 4.3 Result

### 4.3.1 ECM1 Protein Calibration in PBS

### (A) Bode Plot

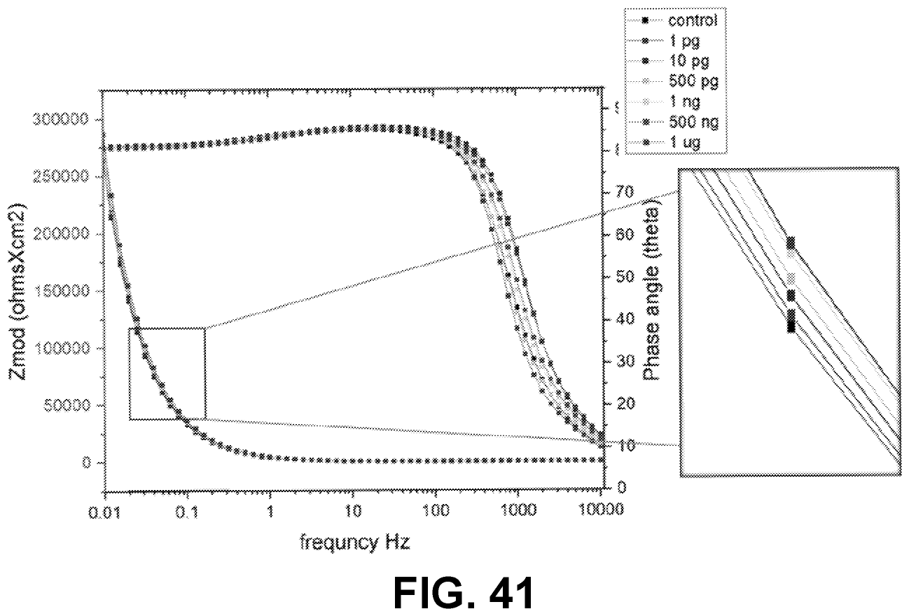

The result shows that the order of the impedance is as follows control <1 pg/ml<10 pg/ml<500 pg/ml<1 ng/ml<500 ng/ml<1 pg/ml ECM1 (FIG. 41). The impedance of ECM1 concentrations are in the order of increasing. The bode plot shows that the data is very consistent.

### (B) Change in Impedance

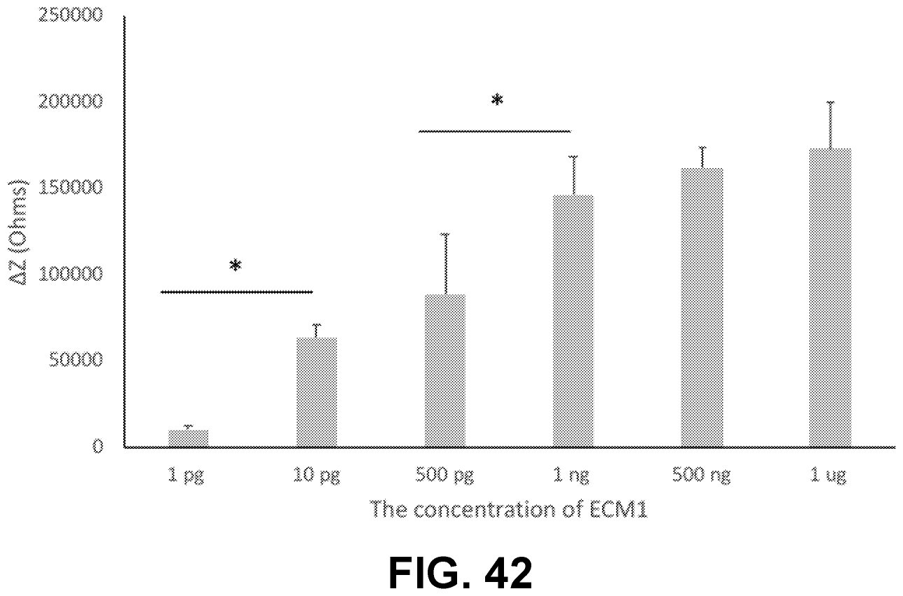

From the bode plot, the change in impedance is tested at 0.0125 Hz. The result shows that (FIG. 42) the change in the impedance Δz increases from 1 pg/ml to 1 μg/ml. There is significant difference in Δz's between the ECM1 concentrations studied.

### (C) Change in Capacitance

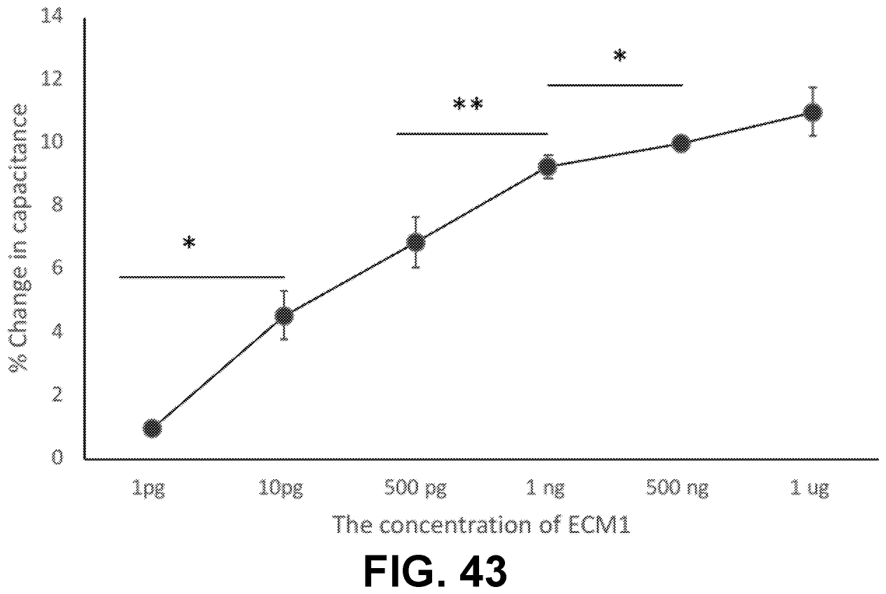

From the CPE circuit, the change in capacitance is tested at 0.0125 Hz. The result shows that (FIG. 43) there is a linear increase in the % change in the capacitance from 10 pg/ml to 1 μg/ml ECM1. The data is consistent because the graphs show a clear increase in percentage changes in capacitance. There is significant difference change in capacitance between the ECM1 concentrations studied except between 10 pg/ml and 500 pg/ml.

### 4.3.2 ECM1 Protein Calibration in Urine

### (A) Bode Plot

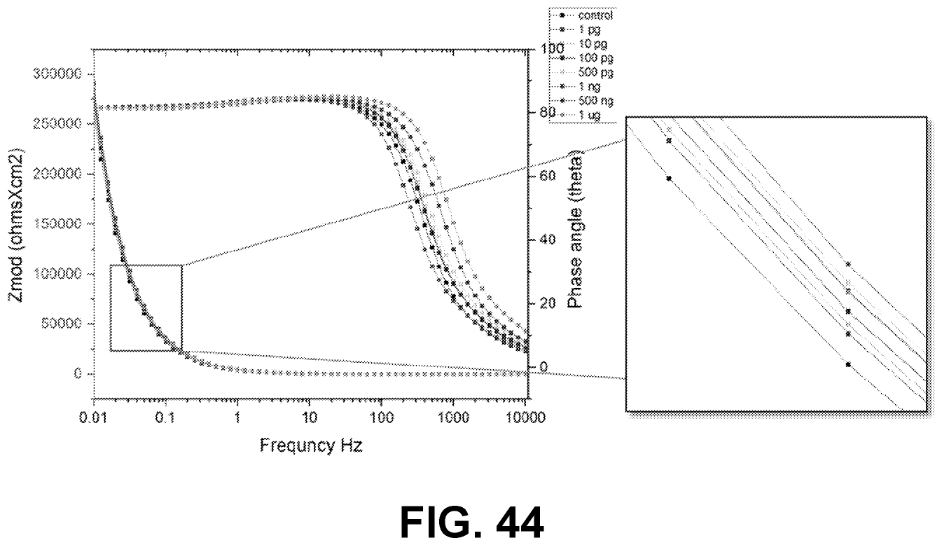

The result shows that the order of the impedance is as follows control <1 pg/ml<10 pg/ml<100 pg/ml<1 ug/ml<500 ng/ml<500 pg/ml<1 ng/ml ECM1 (FIG. 44). The impedance of ECM1 concentrations are in the order of increasing from 1 pg/ml to 1 ng/ml.

### (B) Change in Impedance

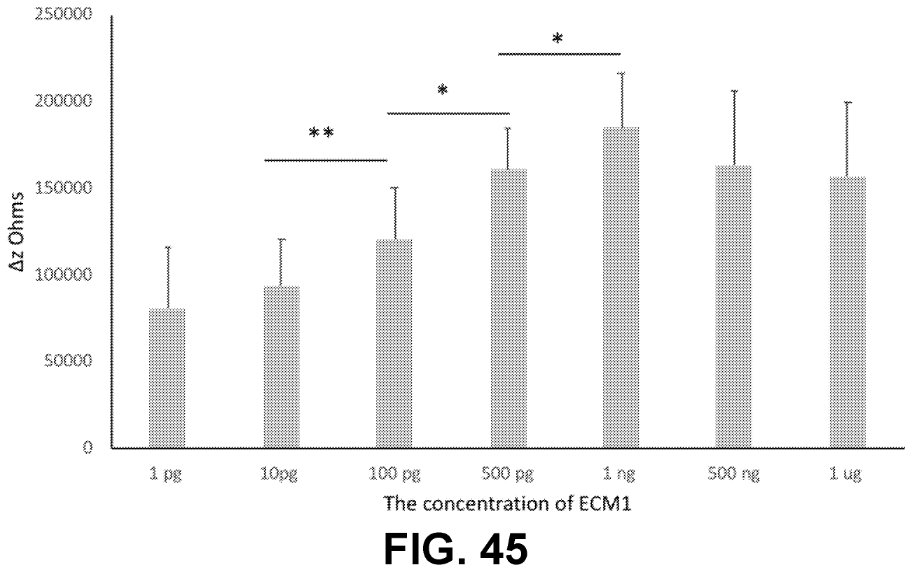

From the bode plot, the change in impedance is tested at 0.0125 Hz. The result shows that (FIG. 45) the change in the impedance increases from 1 pg/ml to 1 ng/ml, then it decreases from 500 ng/ml to 1 μg/ml. A clear difference between Δz's for the ECM1 concentrations from 1 pg/ml to 1 ng/ml. It can be concluded that the detection range of the biosensor for the ECM1 in the urine 1 pg/ml-1 ng/ml.

### (C) Change in Capacitance

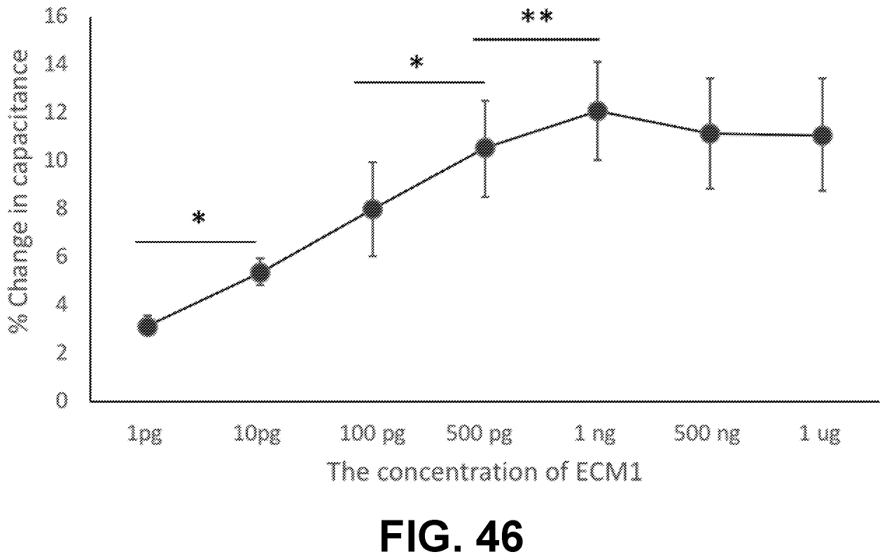

From the CPE circuit, the change in capacitance is tested at 0.0125 Hz. The result shows that (FIG. 46) there is a linear increase in the % change in the capacitance from 10 pg/ml to 1 ng/ml ECM1, and the linear decrease in 500 ng/ml. Thus, the detection range of the biosensor for the ECM1 in the urine 1 pg/ml-1 ng/ml.

### 4.3.3 ELISA

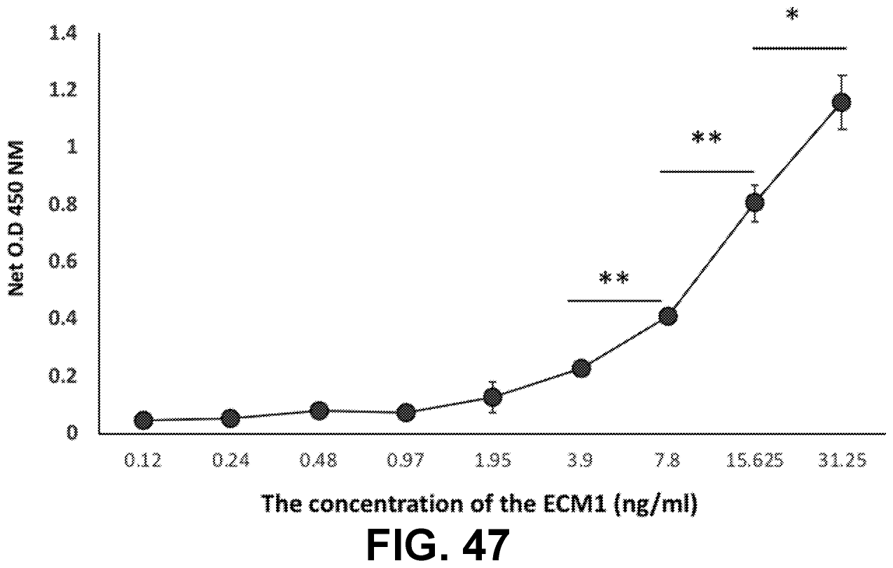

ELISA was performed to compare the detection level of ECM1 between the biosensor tool and ELISA method. Indirect ELISA was performed to detect the ECM1 protein. A serial dilution of the ECM1 protein was performed to know the detection level of ELISA for ECM1 protein. The result shows that the protein increases at 4 ng/ml and above. There is no significant difference in This means that the detection range of ELISA is above 4 ng/ml (FIG. 47).

### 4.4 Discussion

The calibration dose-response study experiment is important for the development of biosensor to calibrate the antigen on the sensor and to detect the detection range of the biosensor for a specific antigen. Many studies have been done for the development of the biosensors in different aspects and application. Most of these studies performed calibration dose-response study experiment.

Previously, portable nanoporous electrical biosensor has been reported for ultrasensitive detection of Troponin-T. The calibration curve was built for detection of Troponin-T. Serial dilutions of purified cTnT in concentrations of 10,000 pg/ml, 1000 g/ml, 100 pg/ml, 10 pg/ml, 1 pg/ml, 0.1 pg/ml, 0.01 pg/ml and 0.001 pg/ml were prepared by diluting stock antigen in 7% BSA. The measured value of impedance (Zmod) was used for estimating the sensor analytical performance. The result showed that the biosensor had a linear range between 0.001 pg/ml and 10,000 pg/ml with a limit of detection at 0.01 pg/ml in 7% BSA.

Another study was done for flexible molybdenum electrodes towards designing affinity based protein biosensors. The calibration dose-response study experiments were performed for a frequency range of 100 mHz to 1 mHz with 10 mV voltage. The cTnl antigen concentration from 100 fg/mL to 10 pg/mL in PBS medium and from 100 pg/mL to 10 pg/mL in the HS medium were tested. The measured value of % change in impedance at 1 Hz frequency was used for estimating the sensor analytical performance. The result showed that the biosensor had a linear response over a broad detection range from pg/mL to u g/mL in PBS and the HS medium.

From these two studies and many other studies, the calibration curve is very important for this study, so aim 2 was to calibrate the ECM1 protein detection using the electrochemical biosensor. The calibration curve indicates the detection range and limit of the sensor for ECM1 in the urine and PBS. In addition, a calibration curve is like a system to determine the concentration of the unknown concentration of the ECM1, so it can be estimated the unknown concentration of the ECM1 in a blind test or the patients' urine samples in the future by the calibration curve.

The calibration dose-response study experiment was performed by tested different concentrations of the ECM1 protein by the electrochemical test to calibrate standard curve for ECM1. For EIS, the frequency range was 10 KHz to 0.01 Hz with 10 mV voltage. ECM1 concentration from 1 pg/mL to 1 μg/mL in PBS medium and artificial urine were tested. PBS solution was used to optimize the biosensor. The measured value of % change of capacitance and change in impedance at 0.01259 Hz frequency was used for estimating the sensor analytical performance.

The results show that there is a linear increase in capacitance and impedance both in PBS solution and synthetic urine as a function of the ECM1 protein. However, the higher degree of variations in the capacitance between the adjacent concentrations of ECM1 protein were observed in synthetics urine compared to PBS samples. This difference can be attributed to the variation in the conductive property of PBS and synthetic urine and subsequent electrochemical responses. Moreover, the detection range of ECM1 in the PBS is from 1 pg/ml to 1 ug/ml. However, the detection range of ECM1 in the urine solution from 1 pg/ml to 500 ng/ml. the detection limit of the sensor for ECM1 is 1 pg/ml in both the PBS solution and synthetic urine.

When calibration curve was done, a serial dilution wasn't performed for ECM1 protein. The concentrations that were tested including 1 pg/m, 10 pg/ml, 100 pg/ml, 500 pg/ml, 1 ng/ml, 500 ng/ml and 1 pg/ml. Then, the calibration curve was converted to log scale, so that will be like a serial dilution from 1 pg/ml to 1000 pg/ml. This calibration curve of the change in impedance will give the accurate value of the unknown concentration of the ECM1 in a blind test or patients' sample urine.

In fact, there is no literature mentioned the level of the ECM1 in the urine. However, there are several literatures studied the ECM1 in the serum and measured the normal level of the ECM1 and the level of ECM1 in the breast cancer in the serum. It has been reported that in normal controls, levels of ECM1 were 5.26-1.85 ng/ml assessed by luciferase-based sandwich ELISA and 5.19-2.43 ng/ml by HRP-based sandwich ELISA. On the other hand, in patients with breast cancer, levels of ECM1 were 11.60-3.77 ng/ml assessed by luciferase-based sandwich ELISA and 10.60-5.19 ng/ml by HRP-based sandwich ELISA. Based on this study, it is expected that the level of ECM1 in the urine will be within the detection limit of the study because the ECM1 goes to the urine, it will be filtered by the kidney, and the amount of the ECM1 in the urine will be in pg/ml. As a result, this indicates that the sensor has a potential to identify the breast cancer at the early stage of development or progressive recurrence with variation in the level of ECM1 protein from the patient's urine.

After that, biosensor sensitivity was compared with another traditional method like ELISA. Most of the studies of the biosensors are used ELISA method to compare or to confirm the results of the biosensor with ELISA. It has been reported that an electronic biosensor can be used for glycan biomarker detection. They used a conventional laboratory technique which is ELISA to compare the performance of the NanoMonitor. The result indicates that the NanoMonitor results is correlated with ELISA results very well. Thus, they found that the detection sensitivity of NanoMonitor is more sensitive than ELISA. Based on this study, the ELISA method was performed to compare between biosensor and ELISA for the detection limit of the ECM1 and to compare the sensitivity between the two methods.

Serial dilution of the ECM1 in the concentration of 1000 ng/ml to 0.12 ng/ml was performed. From the result, there was no increases from 0.12 to 3.9 ng·ml ECM1, then there was a linear increase above 4 ng/ml. From the result, ELISA is not sensitive for the concentrations under 4 ng/ml, and it only can detect the protein with 4 ng/ml concentration and above. Thus, the sensitivity and the detection range of ECM1 by using ELISA is above 4 ng/ml.

In conclusion, the detection sensitivity of an electrochemical biosensor is better than ELISA. It can then be indicated that from these results that the electrochemical biosensor can detect ECM1 until 1 pg/ml, but ELISA is just able to detect the ECM1 with 4 ng/ml or above.

### 4.5 Summary

The results revealed that the detection range of ECM1 in the PBS is from 1 pg/ml to 1 μg/ml, and the detection range of ECM1 in the urine solution from 1 pg/ml to 500 ng/ml. In contrast, the detection range of ELISA is above 4 ng/ml. Hence, it can be concluded that electrochemical biosensor might be a better tool to detect ECM1 from urine.

### Validation of the Biosensor

### 5.1 Materials

Gold screen-printed Electrodes were purchased from DropSens (USA); Extracellular matrix protein-1 (hECM1) (Conc 50 μg/500 μl) was purchased from R & D system; Anti-hECM-1 (affinity purified sheep IgG) (Conc 25 μg) was purchased from R & D system (USA); Dithiobis succinimidyl propionate (DSP) was purchased from Thermo Scientific (USA); Phosphate buffered saline (PBS, PH 7.4) was purchased from Gibco (USA); electrochemical impedance device was from Gamry (USA), and distilled water was used.

### 5.2 Methods

### 5.2.1 Blind Test

A blind test is unknown concentration of ECM1. The concentration of the ECM1 in the urine is tested by the electrochemical test, then the data is analyzed by using the standard curve of ECM1 from the urine.

### Modification of the Electrochemical Biosensor

Clean the sensor with 70% alcohol followed by DI water. Coat the sensor with 10 μl DSP (only working electrode) for 30 minutes in the room temperature, then aspirate the DSP. Incubate the sensor with 40 μl of hECM1 antibody 7 μg/ml (all electrodes) for 30 minutes at room temperature. Aspirate the AB and wash the sensor 1× with PBS. Incubate the sensor with 40 μl of 5% BSA for 30 minutes at room temperature. Aspirate the BSA and wash the sensor 1× with PBS.

### Experiment Conditions

After BSA step, the sensor was incubated with urine (control) for 15 minutes, then EIS is obtained. Then, the urine including ECM1 protein is injected into the chip for 15 min. Then, the EIS is obtained.

### Electrochemical Measurement

The electrochemical tests consist of an open circuit potential (OCP) for 600 seconds, Electrochemical impedance spectroscopy (EIS) at Eoc with an amplitude of 10 mV and frequency of range of 10,000 Hz to 0.01 Hz, and an open circuit potential (OCP) for 100 seconds. From EIS data, the change in impedance (ΔZ) is analyzed at 0.0125 Hz, and CPE circuit is used to test the % change in capacitance.

### 5.3 Result

### 5.3.1 Standard Curve for the Blind Test

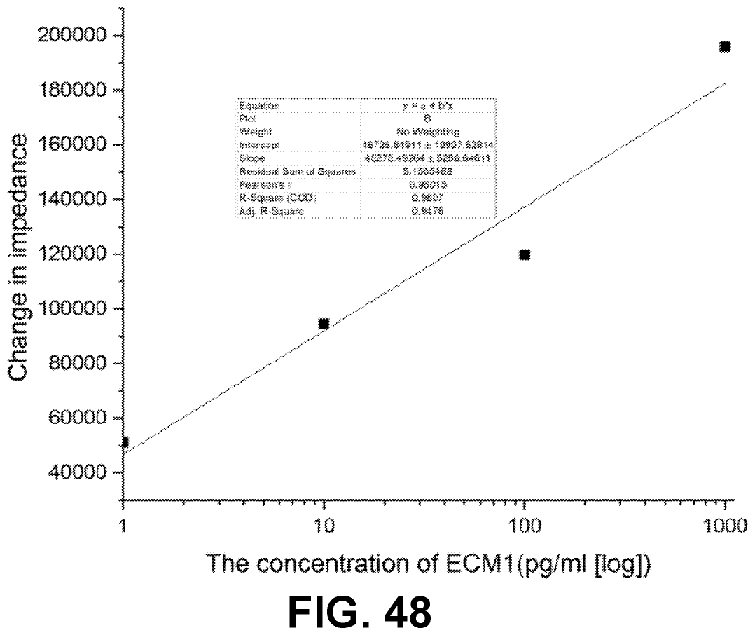

From the standard curve (FIG. 48), the concentration of the blind test or patients' samples in the future can be figured out by estimating the concentration of the ECM1 from the change in impedance standard curve of ECM1 in the urine. X-axis is the concentration of the ECM1 pg/ml by the log. The Y axis is the change in impedance ΔZ. By using Origen software, the concentration of the blind tests can be determined.

### 5.3.2 Blind Tests

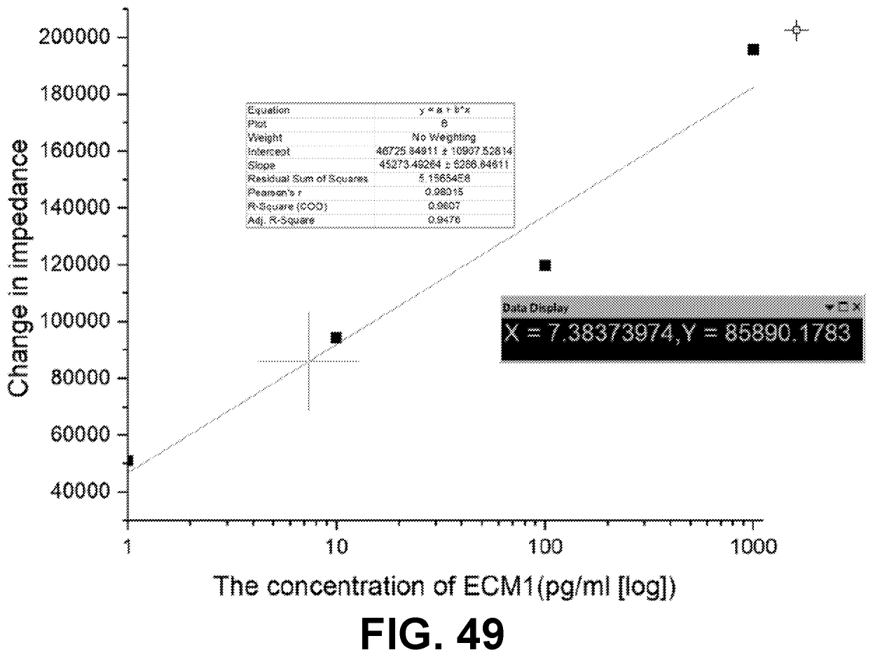

Three blind tests of the ECM1 in the urine were performed. The first test (FIG. 49), the change in impedance was 85956 ohms. Then, from the standard curve, the estimation of ECM1 concentration of blind test 1 was 7.4 pg/ml. The real concentration was 10 pg/ml Ecm1.

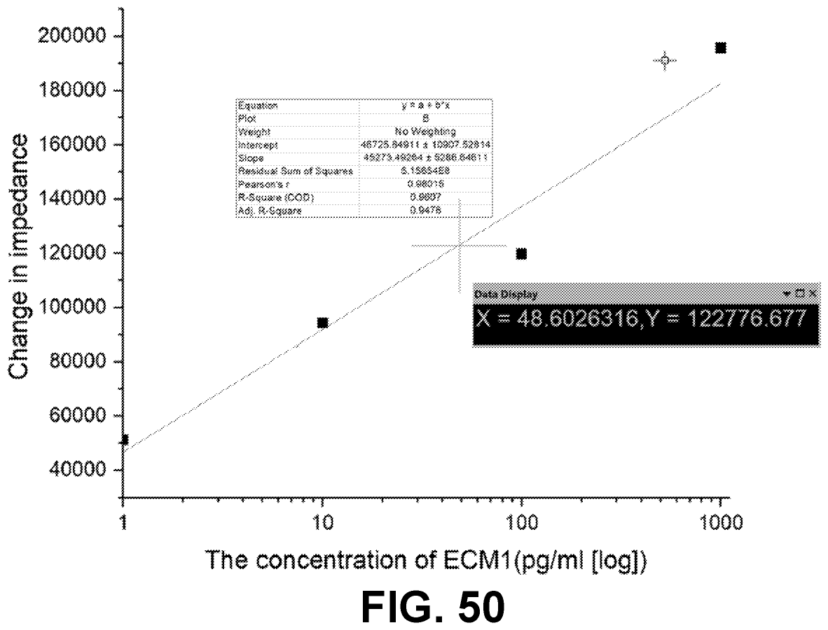

The second test (FIG. 50), the change in impedance was 122550 ohms. Then, from the standard curve, the estimation of ECM1 concentration of blind test 2 was 48.6 pg/ml. The real concentration was 50 pg/ml Ecm1.

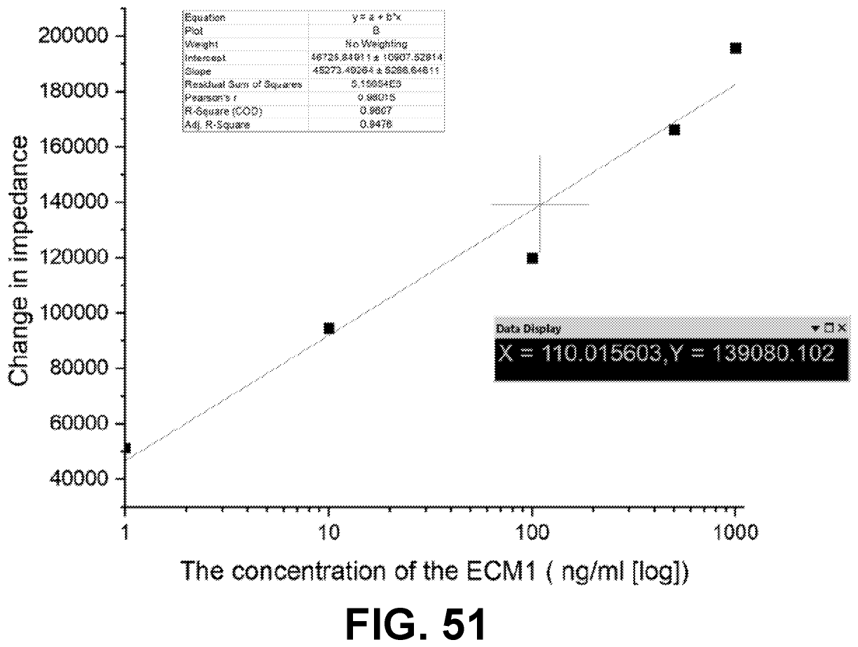

The third test (FIG. 51), the change in impedance was 138870 ohms. Then, from the standard curve, the estimation of ECM1 concentration of blind test 3 was 110 pg/ml. The real concentration was 200 pg/ml Ecm1.

### 5.4 Discussion

After developing the protocol for modifying the biosensor, the data was very consistent in the aim 1. Then, calibrate the biosensor by doing calibration curve for different concentrations of ECM1 in the artificial urine. As mentioned in the previous section, serial dilution of ECM1 was carried out. The calibration curve was plotted in log scale, and the R square was 0.96. After converting the curve to log scale, the calibration curve can be used to estimate the unknown concentration of the ECM1 in blind tests or patients' samples. In addition, in the future, when handle hardware designs, it will be programed to have the software that gives the change in impedance of ECM1 from the standard curve.

Three blind tests were performed, unknown concentrations of the ECM1 were given. First, modify the biosensor by following the same protocol of the aim 1. Then, incubate the sensor with plain urine for 20 minutes as a control, then run the EIS. After that, incubate the sensor with the unknown sample for 20 minutes and run EIS. When EIS is over, data is analyzed and the change in impedance is tested to get ΔZ. The calibration curve is used to estimate the concentrations of the samples, origin software is used to help for estimating the ECM1 concentration. In the calibration curve, Y-axis is the change in impedance, and the x-axis is the concentration of the ECM1 (unknown), so by knowing the y-axis, I will figure out the x-axis.

The results showed that, in the first test, the change in impedance was 85956 ohms. Then, from the standard curve, the estimation of ECM1 concentration of blind test 1 was 7.4 pg/ml. The real concentration was 10 pg/ml Ecm1. In the second test, the change in impedance was 122550 ohms. Then, from the standard curve, the estimation of ECM1 concentration of blind test 2 was 48.6 pg/ml. The real concentration was 50 pg/ml Ecm1. In the third test, the change in impedance was 138870 ohms. Then, from the standard curve, the estimation of ECM1 concentration of blind test 2 was 110 pg/ml. The real concentration was 200 pg/ml Ecm1. From table 3, the error constant was calculated. The average of the error constant of the three blind tests was 1.3. This error may be because different biosensors were used for each test, and as mentioned previously, the initial nature of the biosensors is not uniform, so that could affect the results. Then, serial dilution was performed of ECM1 from the standard curve and the change in impedance for each concentration of the ECM1 was estimated. From table 4, predict concentration was multiplied by the average of the error constant to get the actual concentration.

TABLE 3
Blind Tests

| | ECM 1 | | ECM 1 | | |
|---|---|---|---|---|---|
| Z-change from | concentration | | concentration | | |
| standard curve | (predicted) | | (actual) | | EC |
| 85956 Ohms | 7.4 | pg/mL | 10 | pg/mL | 1.3 |
| 122550 Ohms | 48.6 | pg/mL | 50 | pg/mL | 1 |
| 138870 Ohms | 110 | pg/mL | 200 | pg/mL | 1.8 |

| | |
|---|---|
| Average (EC) | 1.3 |

TABLE 4
Serial Dilution of ECM1 for Estimating the Actual Concentrations

| Z-change from a | ECM 1 concentration | | ECM 1 concentration | |
|---|---|---|---|---|
| standard curve | (predicted-graph) X | | (actual) X*EC | |
| 46322.37 | 1 | pg/mL | 1.3 | pg/mL |
| 60517.9 | 2 | pg/mL | 2.6 | pg/mL |
| 74426.8 | 4 | pg/mL | 5.2 | pg/mL |
| 87673.36 | 8 | pg/mL | 10.4 | pg/mL |
| 101072.7 | 16 | pg/mL | 20.8 | pg/mL |
| 115032.55 | 32 | pg/mL | 41.6 | pg/mL |
| 128482.87 | 64 | pg/mL | 83.2 | pg/mL |
| 141423.7 | 128 | pg/mL | 166 | pg/mL |
| 155536.37 | 256 | pg/mL | 332.8 | pg/mL |
| 169547.127 | 512 | pg/mL | 665.6 | pg/mL |

The result indicated that the electrochemical biosensor is very accurate and sensitive tool to detect the breast cancer biomarker ECM1 from the urine. Even though in the third test it was estimated that the concentration of the ECM1 was 110 but the real concentration was 200 because of the error constant, this is an accurate and closer data. Because in the end, when handle hardware is develop, the range of the ECM1 will be taken, not the exact value. For example, 10 pg/ml-200 pg/ml is the low level, 250 pg/ml-400 pg/ml is pre-high level and 500 pg/ml-1 ng/ml is the high level.

### 5.5 Summary

The blind tests results revealed that biosensor could be an effective detectable tool for breast cancer biomarker ECM1 from the urine because it is a sensitive and accurate method.

### CONCLUSION

### 6.1 Conclusions

Biosensor promises to detect the breast cancer biomarkers in the early stages and monitor the cancer biomarkers after treatment of cancer. The serum level of ECM1 protein in the breast cancer patients is 10-12 ng/ml (normal level 0-5 ng/ml). Based on this, it is expected that the level of ECM1 in the urine will be within the detection limit. This indicates that the sensor has the potential to identify the breast cancer at the early stage of development or progressive recurrence with variation in the level of ECM1 protein from the patient's urine. The results revealed that electrochemical biosensor could be an effective tool for the detection of breast cancer biomarker (ECM1) in the early stages from the urine. The electrochemical biosensor can be an efficient alternative for clinical serological diagnosis of breast cancers.

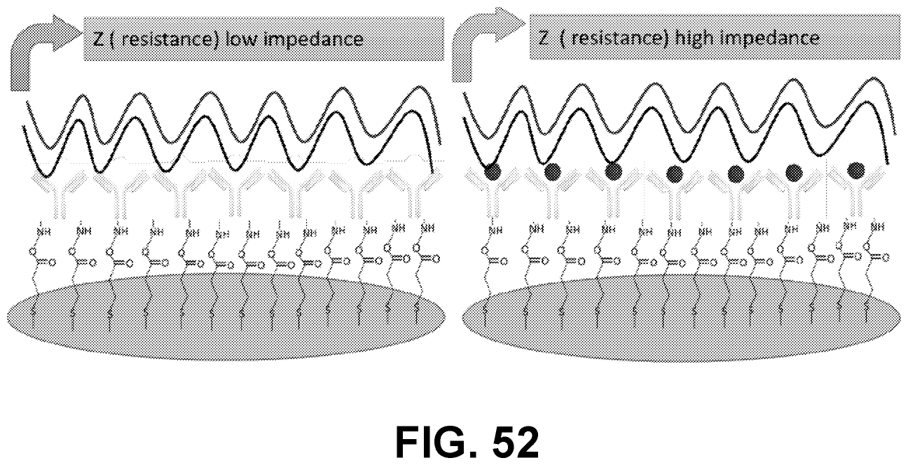

The overall mechanism of the Electrochemical biosensor is that first the biosensor is coated (the gold area) with DSP to immobilize the antibody on the sensor, then the capture antibody is coated on the sensor, Then EIS is run to get The Z1 for the control. The voltage is applied to the sensor to get the current which is impedance Z1. After that, the same sensor is coated with the ECM1, and EIS is run to get Z2 for the test. Also, the voltage is applied to the sensor to get the current which is impedance Z2. It was observed that always Z1 has low impedance, and Z 2 has a high impedance, because as the protein concentration increases the insulating property of the sensor will increase, hence the impedance will increase. Finally, the change of impedance between the control and test is tested. FIG. 52 illustrates this mechanism.

### 6.2 Clinical Implication

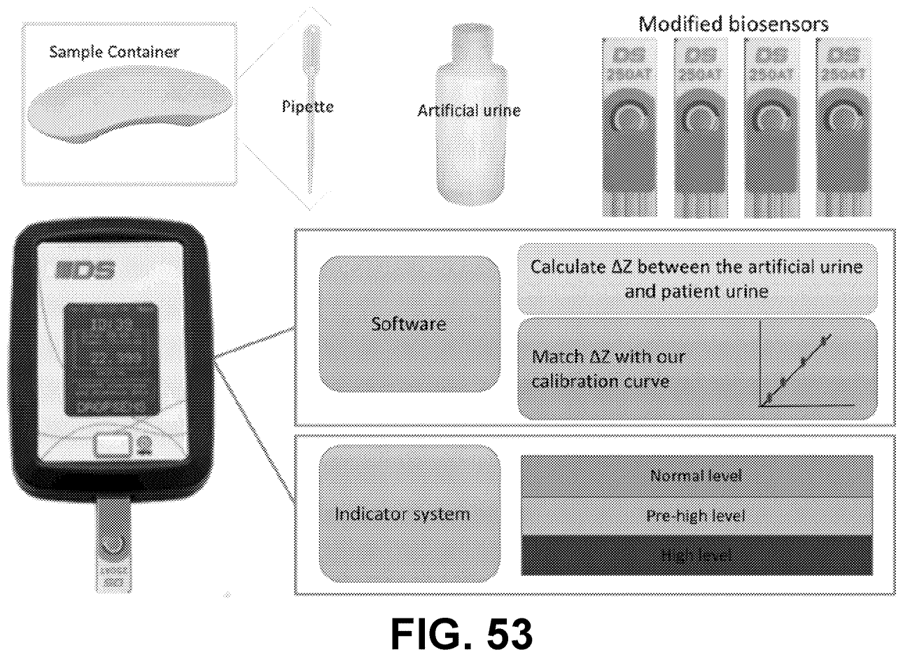

Handheld hardware will be developed to conduct the electrochemical biosensor at home by the patients. This handheld hardware can include 2 components which are software and indicator system. The software contains 2 systems, first system for calculating ΔZ between the artificial urine and patient's urine. The second system for matching ΔZ with the calibration curve of the ECM1 in the urine. In addition, the indicator system is very important for the handle hardware that will indicate the level of cancer. It will have three colors; the green color shows the normal level of the biomarker in the urine. The orange color shows the pre-high level of the biomarker in the urine. The red color shows the high level of the biomarker in the urine. After designing and programming the handheld hardware, it will be tested to make sure it works properly. In addition, besides the handheld hardware, artificial urine, modified biosensor, plate and pipette will be provided. The artificial urine will be used as a control, the plate will be used to collect the urine, and the pipette will be used to drop the urine in the sensor (FIG. 53).

The way for using this product, first, connect the sensor to the hardware and put a drop of the artificial urine in the sensor. Then, the hardware will calculate Z for the control. After that, remove the urine, and collect the patient's urine in the plate, then take a drop by the pipette and add it on the sensor. Hardware will calculate the ΔZ between the artificial urine and patient's urine, and match ΔZ with the calibration curve of the ECM1 in the urine. Finally, the indicator system in the hardware will give the level of the biomarker by the color.

### Example 5—Electrochemical Biosensor (OBSense)—to Detect Childhood Obesity

### Introduction

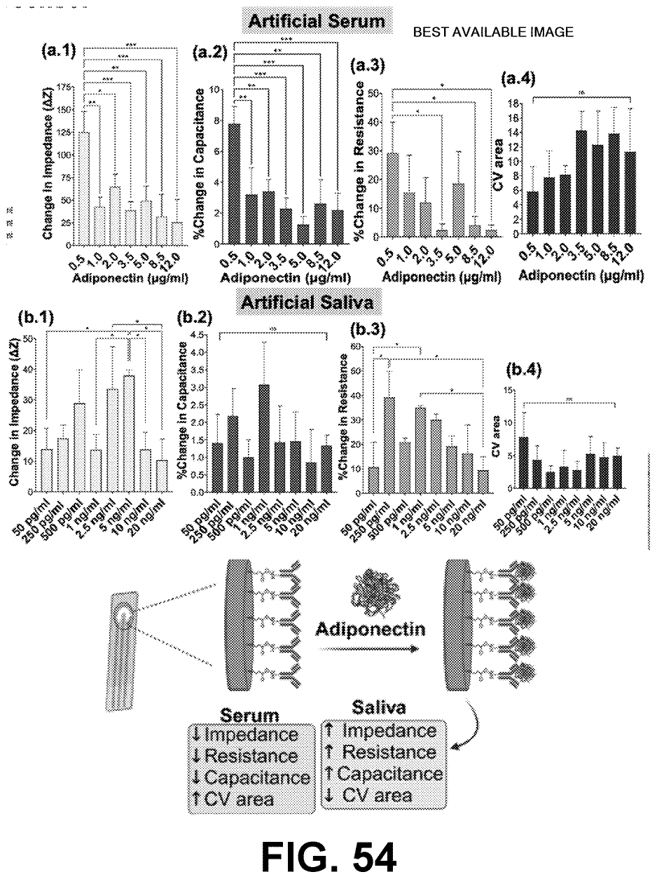

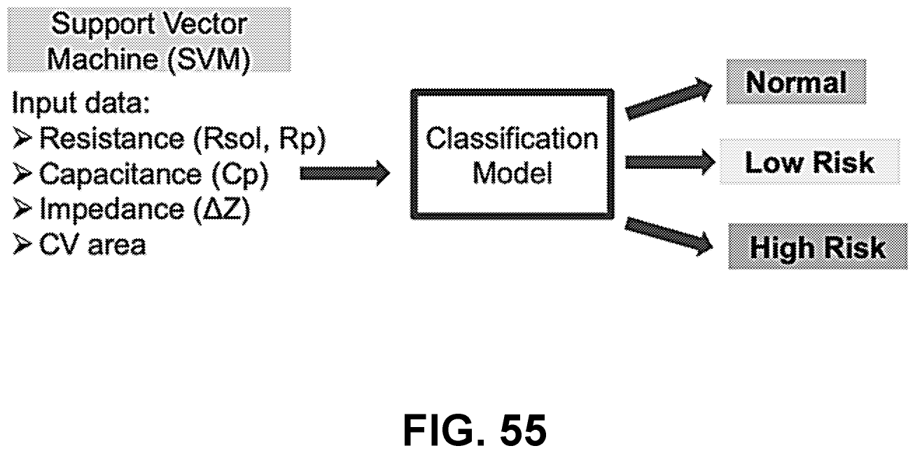

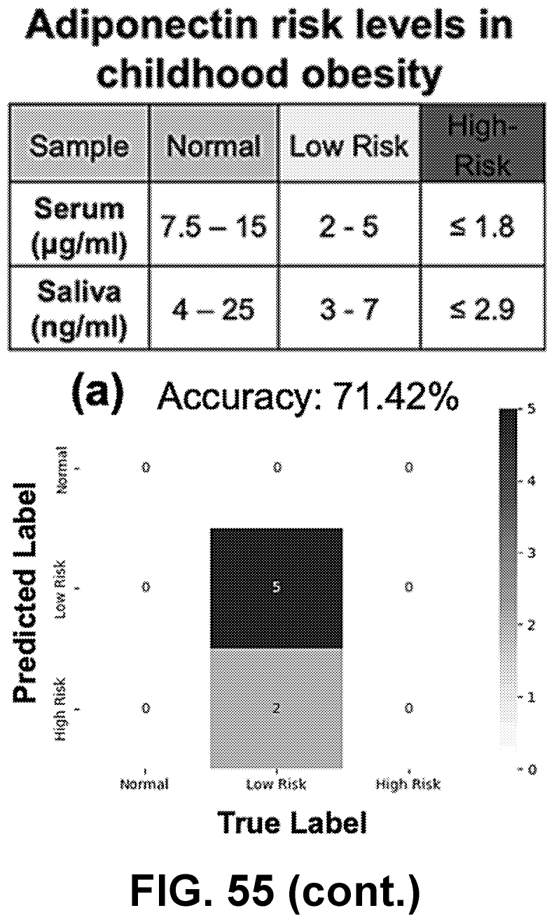

Childhood obesity is a major global public health issue, affecting over 160 million children, with a prevalence of 8.24% worldwide. Adiponectin is the main adipokine secreted by adipose tissue and is known to decrease during obesity. This protein can also be detected in saliva, providing a non-invasive method for application in children. Electrochemical biosensors can improve the detection of metabolic changes associated with childhood obesity, with high specificity, selectivity, and fast response times. Machine learning models can further enhance biosensing with more accurate prediction and classification of adiponectin levels in childhood obesity. Accordingly, an electrochemical biosensor was developed that was able to detect adiponectin in artificial saliva and serum, even at minimal concentrations. FIG. 54 illustrates data generated from protein calibration for artificial serum and saliva while FIG. 55 illustrates a machine learning model trained to predict a risk level in childhood obesity from electrochemical data generated from the serum or saliva.

### Methods

First, an electrochemical biosensor was prepared. The surface of the biosensor was first cleaned with EtOH 70% and treated with DSP for thirty minutes. Then, the biosensor was incubated with the capture antibody solution (anti-adiponectin) for thirty minutes. The biosensor was treated with superblock blocking buffer solution for thirty minutes before a saliva serum was added. Electrochemical analysis was then performed, including EIS and CV.

Next, confocal microscopy and SEM analysis were performed to confirm immobilization of the antibody on the biosensor. Then, the electrochemical data from the electrochemical analysis was input into a machine learning model trained to predict an adiponectin risk level in childhood obesity. For example, a risk could be normal, low, or high depending on the concentration of adiponectin in the fluid sample.

### Discussion

The highest impedance and capacitance values during antibody saturation indicated the full saturation of the electrode surface. The variation observed between artificial serum and saliva calibrations is due to differences in the composition of both fluids and the electrochemical characteristics of adiponectin. Mature adipocytes could secrete adiponectin, replicating normal concentrations to test the biosensor with a range between 5-12 pg/mL. The interaction between adiponectin and the monoclonal anti-adiponectin antibody was confirmed by confocal microscopy and SEM-EDS analysis. An ML model (SVM with RBF kernel) suggested a higher accuracy.

### Conclusion

The developed electrochemical biosensor was able to detect adiponectin in artificial serum, saliva and adipocyte supernatant, even at minimal concentrations. The machine learning models exhibited good accuracy in predicting adiponectin risk levels in childhood obesity.

### Example 6—A Low-Cost and Sensitive Diagnostic Tool to Detect the Earliest Signs of Pancreatic Cancer

Pancreatic cancer is one of the deadliest forms of tumors and it is the fourth most frequent cause of cancer death in the United States. According to the American Cancer Society, the survival rate of people suffering from pancreatic cancer is around 20%, and the five-year survival rate drops down further to 9%. The low survival rate is due to the dismal prognosis of the disease, which is usually exacerbated by the fact that the tumors are diagnosed once they have already progressed to the stage where surgical removal is rendered non-feasible. Moreover, in patients for whom treatment is impossible, the development of cancer can be accompanied by many undesirable side effects such as weakness, weight loss, and pain. Furthermore, for patients who are susceptible to surgical treatment, there is evidence of the recurrence of the tumor after two years of surgery in most cases. The current diagnosis methods for pancreatic cancer rely mostly on imaging techniques like computed tomography, endoscopic ultrasound, and magnetic resonance imaging. There are several limitations of these techniques. However, these clinical tests are expensive, require biopsy, and toxic radiation exposure, and are less available to common people. Therefore, a diagnostic platform for pancreatic cancer which can identify the disease at an early stage could permit timely and curative therapy and monitor recurrence after surgical treatment.

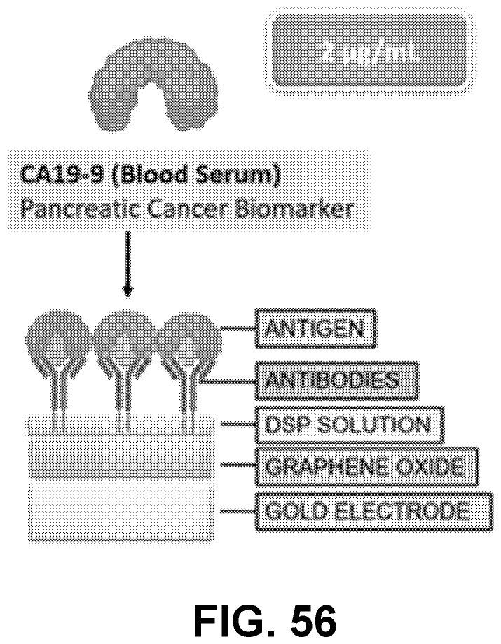

The primary objective of this Example focuses on the development of electrochemical-based biosensors for the early detection of pancreatic cancer (see FIG. 56). In literature, protein-based biomarkers are an attractive identifier for determining the occurrence of pancreatic cancer and have been recognized as precise and efficient diagnostic tools that anticipate the need to uncover the prognostic outcomes for pancreatic patients. Therefore, various aspects of the present disclosure focus on a serum-based electrochemical approach based on the protein biomarkers present in serum samples. In that regard, three major protein-based biomarkers associated with pancreatic cancer have been selected: CA19-9, CA 242, and CEA, as their prevalence and expression levels in serum samples offer information about the presence of pancreatic cancer.

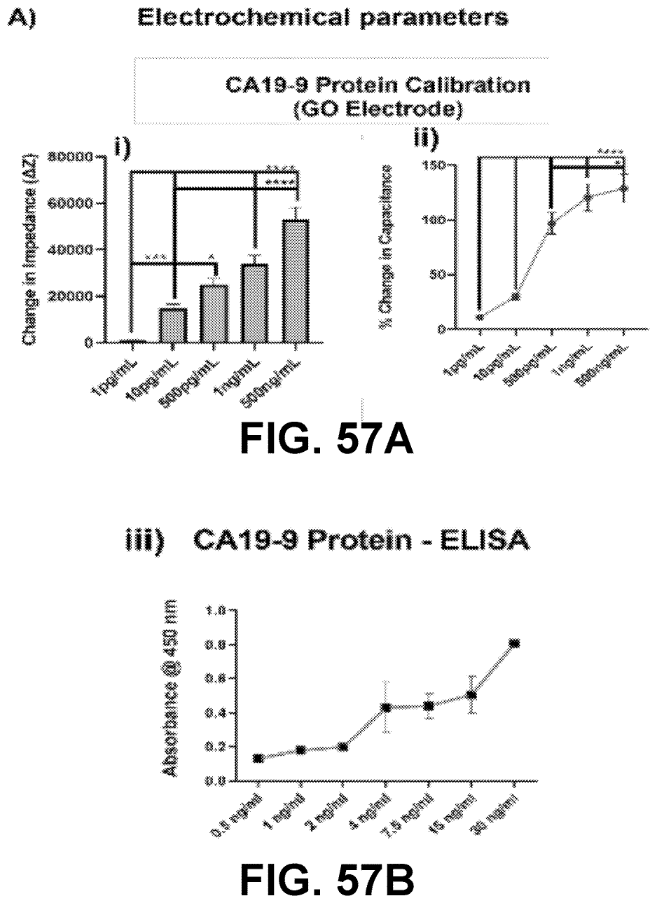

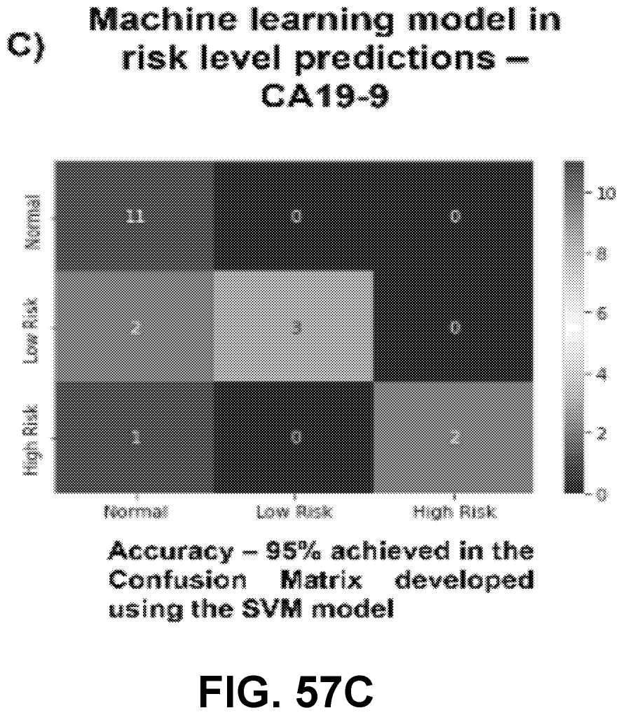

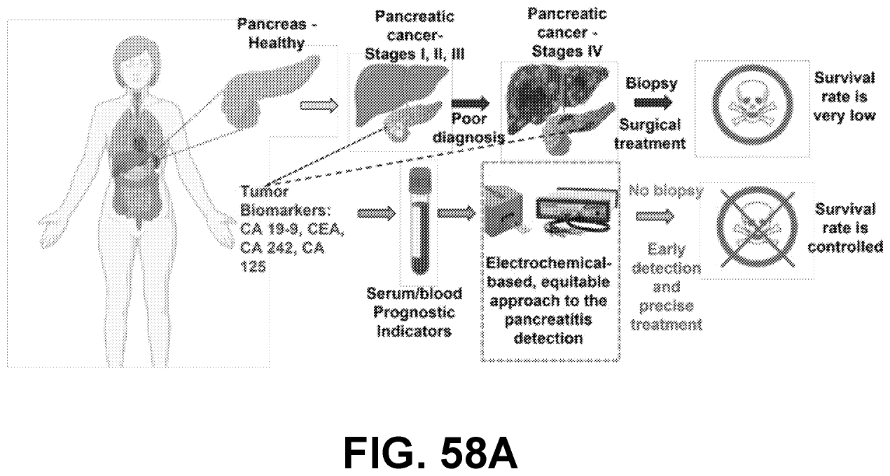

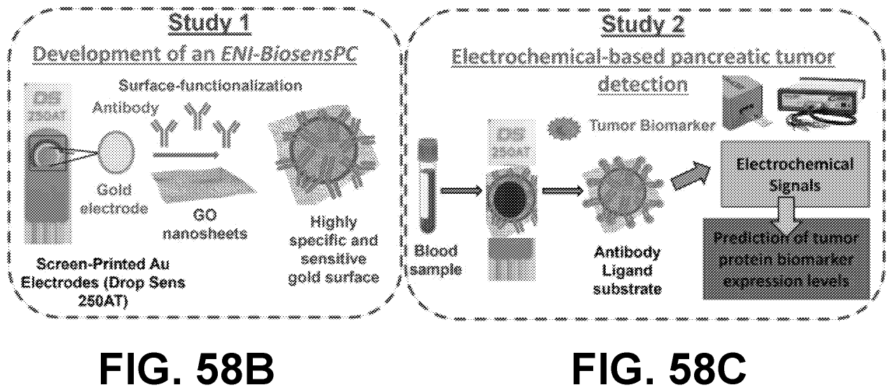

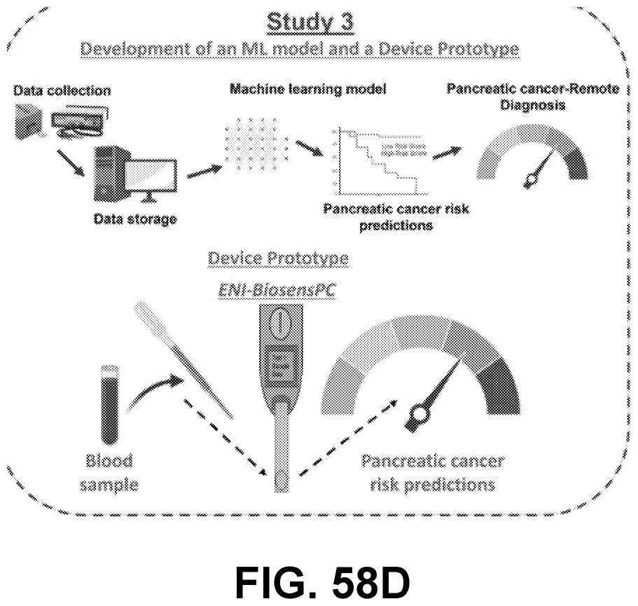

FIGS. 57A-57C and FIGS. 58A-58D illustrate various aspects of an electrochemical biosensor and methods for predicting a risk level of pancreatic cancer.

### Example 7—Electrochemical Biosensor for Detection of Various Diseases Pancreatic Cancer

The uncontrolled growth of aberrant cells within the pancreas, which results in tumor development, is an indicator of pancreatic cancer. The pancreas is located beneath the stomach, and this cancer type is severe and occasionally aggressive. In the United States, pancreatic cancer accounted for the third most common cause of cancer-related mortality for both sexes. According to the American Cancer Society's predictions, 64,050 people will likely be diagnosed with pancreatic cancer in 2023, and this disease will be accountable for 50,550 deaths. Its survival statistics frequently lag those of other malignancies, largely as a result of delayed diagnosis at difficult treatment stages. The illness stage and available treatments have an impact on the five-year survival rate, which ranges from 10% to 15%. Age, smoking, obesity, family history of pancreatic cancer, certain hereditary abnormalities (such as BRCA1 and BRCA2), chronic pancreatitis, and diabetes are all risk factors. Pancreatic cancer early detection or diagnosis is hampered by vague symptoms that may not show up until the disease has advanced. Back or stomach discomfort, unwanted weight loss, jaundice, decreased appetite, digestive issues, and weariness are typical warning signs. Depending on the illness stage, the patient's condition, and preferences, treatment options may include surgery, radiation, chemotherapy, targeted therapy, or immunotherapy. However, current approaches have drawbacks, such as unclear symptoms, ineffective treatments, intrusiveness, false-negative results, and genetic/molecular diversity. Improving patient well-being can depend on the development of an early detection technique.

Electrochemical biosensors (EBs) show promise in the diagnosis of cancer by using electrochemistry to convert biological interactions into quantifiable electrical signals. It also makes it easier to identify pancreatic cancer-specific biomarkers like carbohydrate antigen 19-9 (CA19-9), cancer antigen 242 (CA-242), and carcinoembryonic antigen (CEA), which are specifically useful for identifying cancer biomarkers quickly and accurately.

Real-time, accurate readings from EBs are provided, assisting in the monitoring and early identification of illness. EBs have remarkable sensitivity for detecting minute biomarker concentrations. Using specific recognition elements like antibodies, aptamers, or DNA probes, these biosensors demonstrate exceptional selectivity and may successfully separate cancer biomarkers from interfering substances. Their small size enables point-of-care applications, doing away with the requirement for pricey laboratory machinery. There are difficulties with biosensor stability, repeatability, addressing the complexity of clinical samples, and demonstrating clinical value through electrochemical biosensor integration in cancer detection.

To increase performance, nano-engineered electrochemical biosensors have been developed that use nanotechnology to analyze biological molecules or processes combine the advantages of electrochemical sensitivity with the special features of nanomaterials. The improved sensitivity, selectivity, and stability of graphene oxide (GO) biosensors are made possible using graphene oxide as a sensing substrate. Health care, environmental monitoring, and food safety are among the areas where graphene oxide biosensors are used. They can be used for identifying disease-related biomarkers and enabling sensitive detection for diagnostics, monitoring, and bioanalysis.

### Stroke

Oral health as an indicator of systemic health is becoming increasingly relevant with many diseases like periodontitis having links to systemic diseases. One such systemic condition is stroke. According to CDC every 40 seconds, someone in the United States has a stroke, and it is a leading cause of death for Americans. This study considers periodontitis biomarker MMP9 and oxidative stress biomarker GSH to be used for the early risk prediction of stroke. Electrochemical analysis of artificial Saliva was done using Gamry with increasing concentrations of MMP9 and GSH protein independently. Electrochemical Impedance Spectroscopy (EIS) and Cyclic Voltammetry (CV) were conducted. From the EIS data, the change in capacitance and charge transfer (CV area) was calculated for each test condition. The electrochemical properties obtained could be used together with the resistance of solution (Rsol), polarization resistance (Rp), and constant phase element (CPE) as machine learning data input. Scanning Electron Microscopy (SEM) with Energy Dispersive Spectroscopy (EDS) was employed to observe the change in the electrode surface and elemental composition data present on the sensor surface also showed the antibody is attaching to the biosensor enabling protein detection. ELISA and confocal Imaging enabled biological characterization of MMP9 and GSH antibody-antigen interaction. Machine learning models using MMP9 and GSH electrochemical data were applied for the prediction of risk levels of stroke with periodontitis and salivary oxidative stress. The detection of MMP9 and GSH by electrochemical biosensors indicates the potential to use them as electrochemical biomarkers and use of the data for ML-driven prediction tool for stroke risk.

### Periodontitis

Periodontitis is marked by the inflammation and peeling away of the gum tissue, with bone and tooth loss in advanced stages. It affects nearly half of all Americans, including over 70 percent of the elderly. Periodontitis promotes the host immune response in the form of a complex network of molecular pathways involving proinflammatory mediators such as cytokines, growth factors, and MMPs. The enzyme MMP-9 (matrix metalloproteinase 9) is a core component of the causal pathway behind periodontitis and comprises the most important pathway in tissue destruction associated with periodontal disease. MMPs participate in morphogenesis, physiological tissue turnover, and pathological tissue destruction. However, the alterations in the regulation of MMP activity are implicated in the manifestation of oral diseases. Moreover, MMPs can be considered a risk factor for periodontal disease. To that end, the measurements of MMP levels may be useful markers for early detection of periodontitis and as a tool to assess prognostic follow-ups. Therefore, detection and inhibition of MMPs can be useful in periodontal disease prevention or part of periodontal disease therapy that can greatly improve oral health globally.

Electrochemical biosensors, combined with machine learning, can be viable for the early detection of periodontitis risk, using the biomarker MMP-9. Electrochemical-based biosensors have been developed for the early detection of periodontitis. In the literature, protein-based biomarkers are an attractive identifier for determining the occurrence of oral diseases with precise and efficient diagnoses that anticipate the need to uncover the prognostic outcomes for periodontic patients. The method employs a saliva-based electrochemical approach to predict periodontitis risk using MMP-9 as the prognostic biomarker.

### Breast Cancer

Breast cancer is a significant health issue in the world today. As per 2020 breast cancer statistics, 2.26 million new cases and 685,000 deaths were reported worldwide. Procedures such as magnetic resonance imaging (MRI) and biopsies are commonly used to diagnose breast cancer. However, these techniques are very expensive, time-consuming, and painful for patients. Electrochemical biosensors are new devices capable of providing early and reliable diagnostic results. Early detection of cancer is essential for a patient's longevity: when breast cancer is diagnosed early, a patient's five-year survival rate is 99%. Intelligent biosensors for cancer (iBiosensC) that use machine learning (ML) can further address limitations in biosensor research, such as big data analytics. Therefore, there is a need for a novel breast cancer diagnostic system that renders early and accurate results. Electrochemical biosensors can be used to detect matrix metallopeptidase 9 (MMP-9), a breast cancer protein biomarker, in urine and an ML model can be utilized in early breast cancer diagnosis.

A Python module used to automate electrochemical impedance spectroscopy (EIS) raw data modeling and an ML algorithm for iBiosensC cancer risk prediction were developed. In the experimental study, the electrodes of a sensor were cleaned with 70% isopropyl alcohol and DI water. Next, the entire sensor was coated with a DSP solution (4.041 mg DSP/1 mL DMSO), an antibody solution (5 μg antiMMP-9/1 mL PBS), and TBS Superblock. An MMP-9 solution was then added to the sensor, which was analyzed through EIS and cyclic voltammetry (CV). Lastly, (1) a Python script to model the sensor's equivalent circuit and (2) a support vector machine (SVM) that classifies a patient's risk level into three categories were developed. Using the Bode plot generated for each trial, the change in impedance (ΔZ) at a frequency of 0.0126 Hz was calculated. In artificial urine, an increase in ΔZ as MMP-9 concentration increased was observed. Additionally, higher oxidation peaks for larger concentrations of MMP-9 were found for PBS.

When given the EIS and CV data for the MMP-9 and extracellular matrix protein 1 (ECM-1) biomarkers, the SVM was observed to be about 84% accurate in the prediction of breast cancer risk. A linear increase in impedance is associated with an increase in ECM-1 concentration. As polymers of amino acids, both MMP-9 and ECM-1 insulate electricity, contributing to a decrease in current and increase in impedance. The efficacy of SVMs in the analysis of EIS data was found in a previous study, in which a model accuracy of 98% was observed. In summary, MMP-9 was successfully detected in artificial urine using biosensors, and ML was used to provide accurate risk predictions.

### Childhood Obesity

Childhood obesity has dramatically increased in recent decades and has swiftly become a public health problem in the United States and around the world. The electrochemical sensor of various aspects of the present disclosure detects the presence of the biomarker adiponectin specific to obesity. In combination with machine learning as described herein will quantify the risk level of detected adiponectin and give an early warning with respect to obesity.

### Materials and Methods

Materials: Gold screen-printed Electrodes (DRP-250AT) and graphene oxide screen-printed Electrodes (DRP-110AT) were purchased from DropSens, USA; Graphene oxide nano colloids solution (2 mg/ml, dispersed in water) was purchased from Sigma Aldrich; CA19-9 protein was purchased from R & D systems; CA19-9 monoclonal antibody (1 mg/ml of 100 μg of mouse IgG) was purchased from R & D systems, USA; Superblock (TBS) blocking buffer, Dimethyl Sulfoxide (DMSO) and Dithiobis succinimidyl propionate (DSP) was purchased from Thermo Fischer Scientific, USA; Phosphate buffered saline (PBS, pH 7.4) was acquired from Gibco, USA;). Electrochemical measurements were performed with potentiostat Advanced Electrochemical System Gamry Analyst with licensed Gamry software, USA, and all the reagents used for this study were analytical grade.

Preparation of graphene oxide nanosheet coated biosensor: The gold surface of the gold screen printed electrodes was coated with graphene oxide noncolloidal solution and was exposed to ultraviolet radiation overnight. The UV rays crosslink the coated graphene oxide nanosheet with the electrode.

Electrochemical measurements: Electrochemical measurements were performed with potentiostat Advanced Electrochemical System Gamry Analyst. All experiments were carried out with a three-electrode system that is comprised of a platinum reference electrode, a counter carbon electrode, and a gold working electrode (4 mm) in case of gold screen printed biosensor, a graphene oxide working electrode and graphene oxide nanosheet working electrode in case of graphene oxide screen printed and graphene oxide nanosheet coated biosensors respectively. Electrochemical impedance spectroscopy was used to characterize the affinity between the antibody (anti-CA19-9) and the biomarker (CA19-9) on the electrode surface and cyclic voltammetry was used to measure the redox changes. The electrochemical tests include an open circuit potential (OCP), Electrochemical impedance spectroscopy (EIS) at Eoc with an amplitude of 10 mV and frequency range of 10,000 Hz to 0.01 Hz, and cyclic voltammetry (CV). From the EIS data, the change in impedance (ΔZ) was analyzed at different frequencies to obtain the optimum frequency.

$$Δ Z = impedance of the test - impedance of the control$$

Modified Randle's circuit model was used to fit the impedance spectra and identify the electrochemical nature of the bond, leading to the classification of specific versus nonspecific interactions on the microelectrodes and testing the % change in capacitance and resistance.

$$\% Change in Capacitance = \frac{Capacitance of the test - Capacitance of the control}{Capacitance of the control} * 100$$

$$\% Change in Resistance = \frac{Resistance of the test - Resistance of the control}{Resistance of the control} * 100$$

Based on cyclic voltammetry data, the area under the curve was calculated

$$\% change in area = \frac{area of the test - area of the control}{area of the control} * 100$$

Antibody saturation: To determine the optimal antibody concentration required to saturate the biosensor for the detection of biomarkers CA19-9: The gold, graphene and graphene oxide coated electrochemical biosensors were cleaned using ethanol and incubated for 30 minutes with 10 μl of 10 mM DSP dissolved in 1 ml of DMSO. DSP is a NHS ester-based homo-bifunctional crosslinker which helps in binding the analytes to the surface of the biosensor The crosslinker treated biosensor was inserted into the Gamry 1000 potentiated and 40 μl of antibody against the proteins CA19-9 diluted in PBS was added. The different cumulative concentrations of CA19-9 antibody ranges from 0, 0.5, 1, 2, 3.75, 7.5 and 15 were tested. Post half an hour incubation electrochemical impedance spectroscopy and Cyclic voltammetry were performed and the collected data was analyzed using the Simplex method of the Gamry software. Based on this the percentage change in Capacitance, resistance, and change in impedance was calculated.

Calibration and characterization of sensor: The procedure was followed until coating the biosensor with antibody. Post 30 minutes incubation the biosensor was washed twice with 40 μl of PBS to remove unbound antibody and then the sensors was incubated for half an hour with 40 μl of TBS blocking buffer to block nonspecific bindings. After blocking the sensor, it was treated with PBS (control) and PBS containing CA19-9 protein, and the electrochemical analysis (EIS and CV) was performed. Different concentrations such as 1 pg/ml, 10 pg/ml, 100 pg/ml, 500 pg/ml, 1 ng/ml, 500 ng/ml, and 1 μg/ml of PBS containing CA19-9 were tested.

Biological Characterization (ELISA): The 96 well plates were coated with 100 μl of biomarker CA19-9 in varying concentrations for overnight at 4° C. Following washing twice with TBST and once with TBS, 100 μl of blocking agent was added to block the non-specific binding for 2 hours. Post washing 100 μl of CA19-9 antibody was added and incubated for 1 hour. Similarly secondary antibody conjugated with HRP was added and incubated. Finally, after washing, the TMB substrate solution was added, and the color developed. A stop solution (sulfuric acid) was added to restrict the reaction from proceeding. The absorbance was measured as 450 nm.

Python module to automate the steps for analysing the EIS and CV data: A python code was developed based on the simplified Randles circuit model, which performs similar to gamry software in order to calculate the EIS data like capacitance and resistance. In addition to that the area under the trapezoid function was used to calculate the area under the curve in CV plot.

AI model to predict pancreatic cancer: Support vector machine method was developed to predict the risk levels of pancreatic cancer based on the ranges on the biomarker. The values of capacitance, resistance and impedance calculated based on EIS and area under the curve of CV plots obtained manually and from automation code were fed as input data into the model. 80 percent of the data was used for training while the remaining was used for testing. The output was obtained as a heat map with the parameters being risk levels (Normal, Low risk and High risk).

Blind Test: A blind test was performed by using the unknown concentration of CA19-9 protein in PBS. The electrochemical test tested the concentration of CA19-9 in PBS; then, the data was analyzed using the standard curve of CA19-9 diluted in PBS.

Statistical analysis: The data distribution will be initially assessed using a histogram and P-P plot for visual inspection. Subsequently, the normality of the data will be determined using the Shapiro-Wilk test. If the Shapiro-Wilk test is significant, skewness and kurtosis will be used to assess if the data were approximately normally distributed. After confirming that the data is normally or approximately normally distributed, a one-way ANOVA will be conducted to compare differences; post hoc tests will be performed to further analyze the differences between specific groups. Specifically, Dunnett's test will be used to compare the means of the groups of interest with the mean of the reference group, while Tukey's test will be applied for all possible pairwise comparisons. Dose-response relationships will be further examined using linear regression models. The statistical significance level will be set at p<0.05 and SAS 9.4 (SAS Institute Inc, Cary, NC) will be used for statistical analysis.

### Results

### Electrochemical Experiments for Antibody Optimization and Protein Calibration Using Different Electrodes—Gold, Graphene-Coated, and Graphite Electrodes:

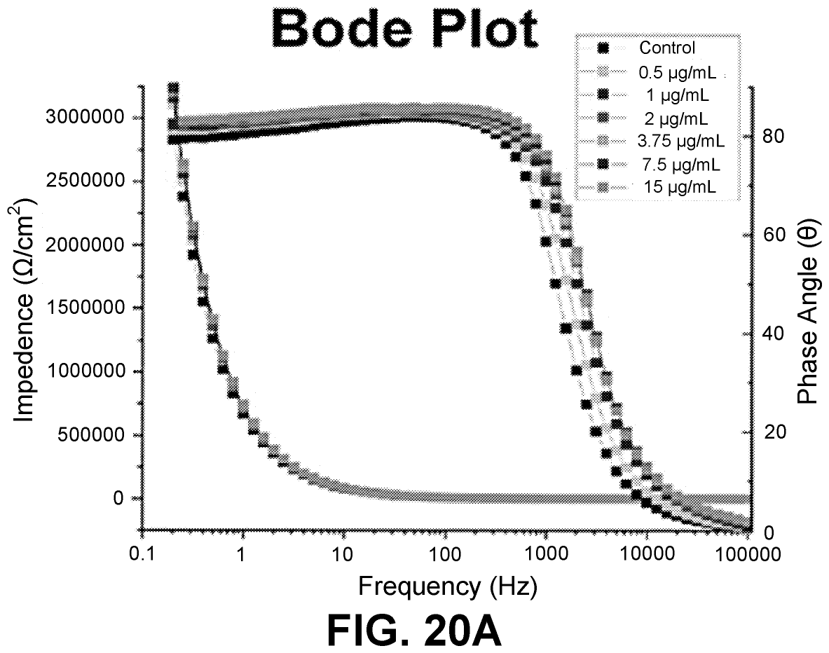

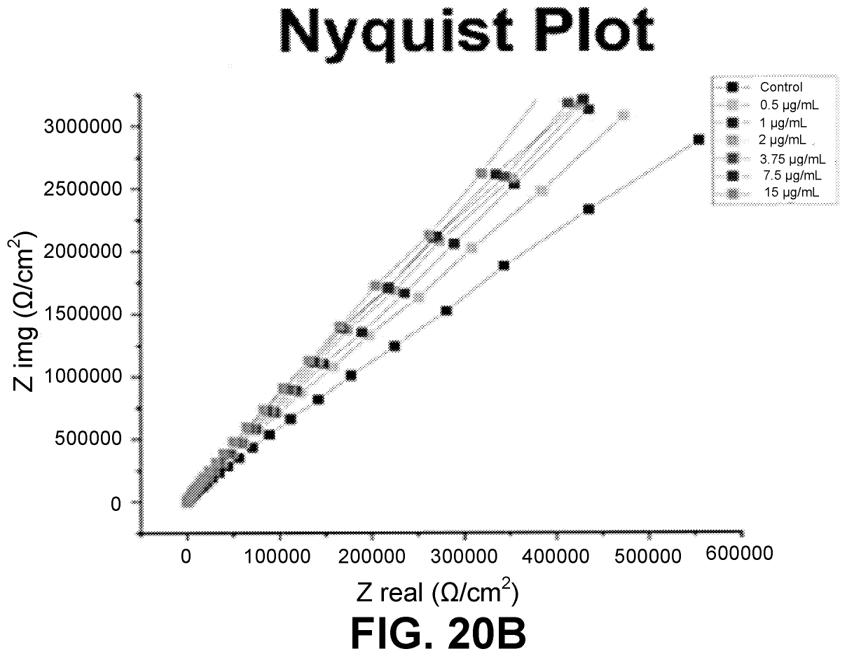

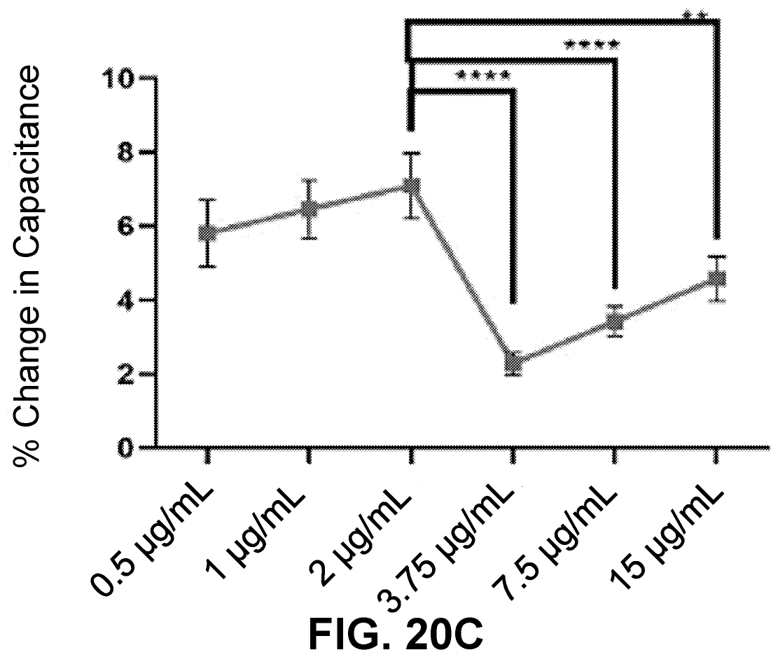

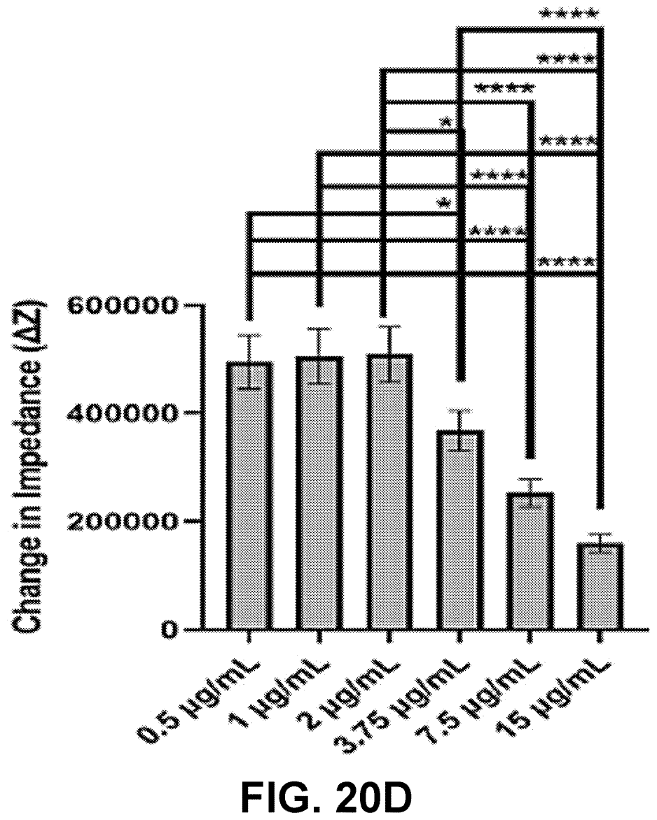

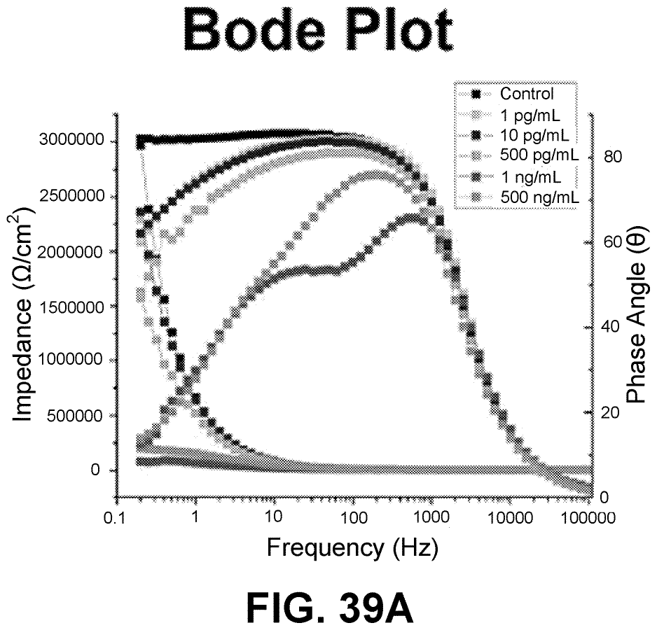

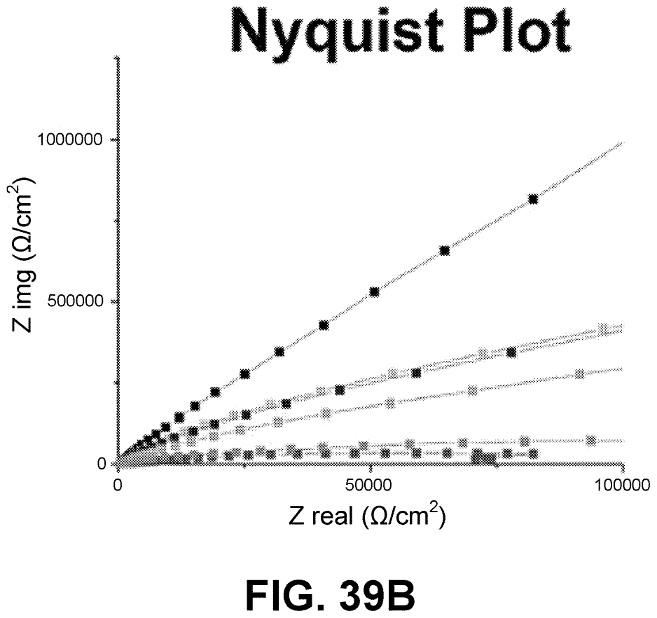

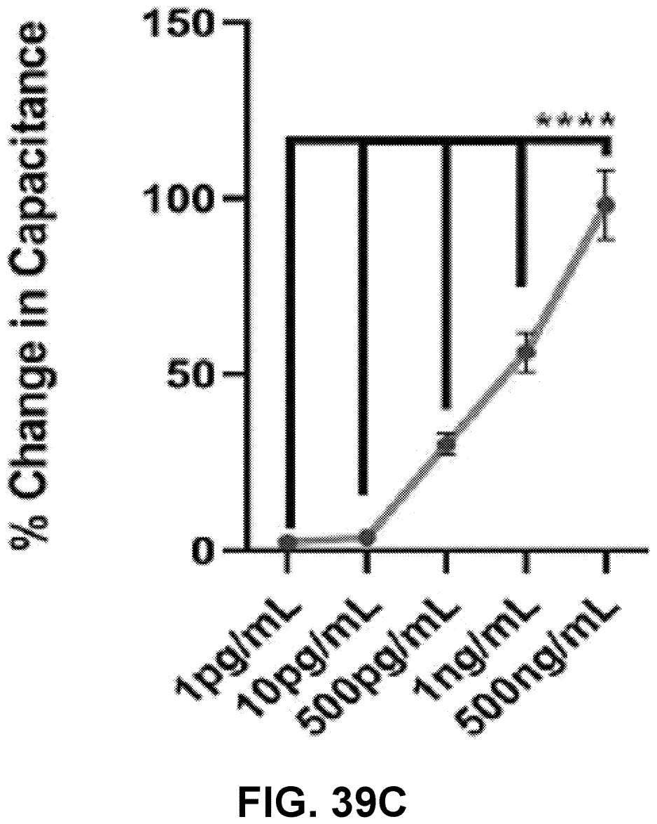

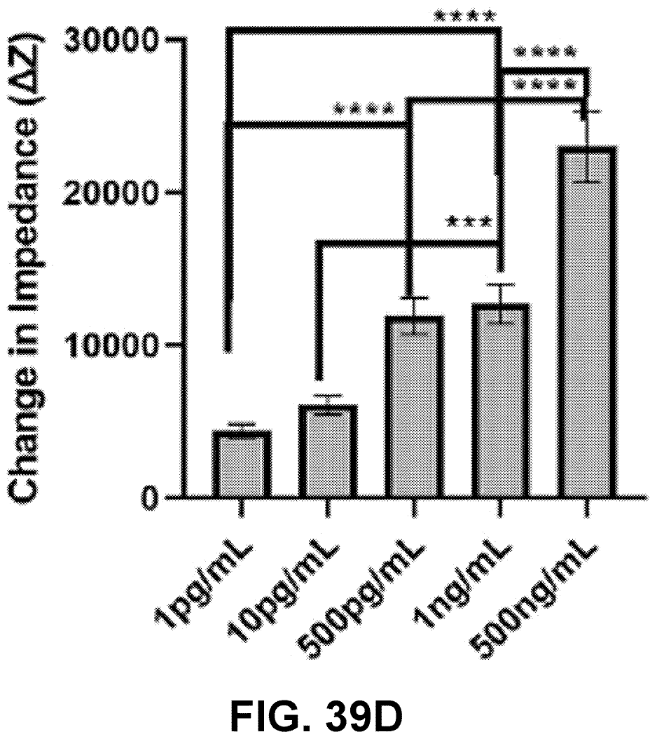

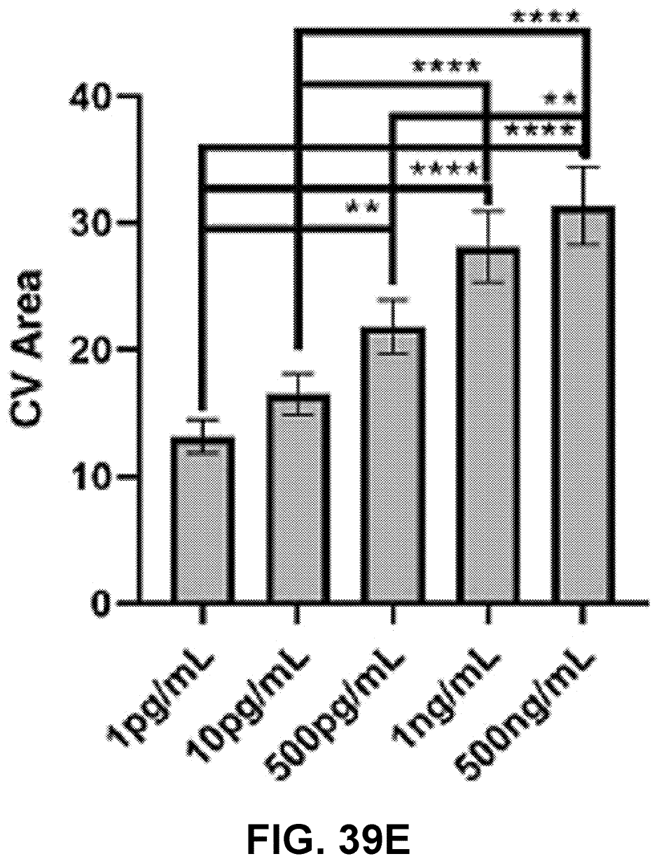

Through the electrochemical antibody saturation and protein calibration experiments, each repeated with the gold, graphene-nanoparticle-coated, and graphene electrode, there was an observable difference in each electrode's sensitivity to varying CA19-9 protein concentrations. The gold electrode provided baseline electrochemical values that were used for comparison to test the effects of graphene oxide on the electrochemical biosensor sensitivity. Initially conducting the antibody saturation experiments with the gold electrode to identify the optimal concentration of antibody to use for protein detection, both impedance and capacitance graphs (FIGS. 20A-20D and 39A-39E) indicated that 2 μg/mL was the optimal concentration of antibody to utilize in the protein calibration experiments. FIGS. 20A-20D depict the electrochemical data from the antibody saturation experiments with the gold electrode. After identifying an optimal concentration of CA 19-9 antibody, protein saturation experiments were conducted using the gold electrodes, and the corresponding electrochemical data are presented in FIGS. 39A-39E. The accurate distinction of different CA19-9 protein concentrations was confirmed with statistically significant differences in CV area, change in impedance, and percent change in capacitance values between different protein concentrations. The resulting Nyquist and Bode plots confirmed the distinct detection of different CA 19-9 protein concentrations.

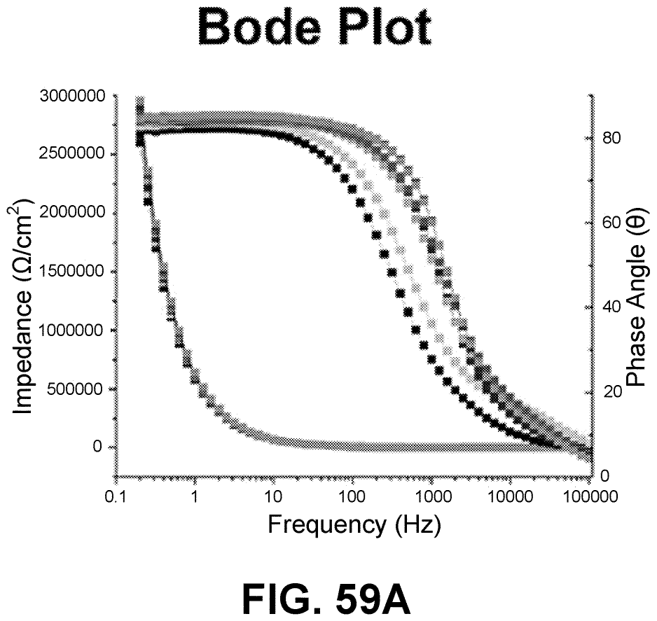

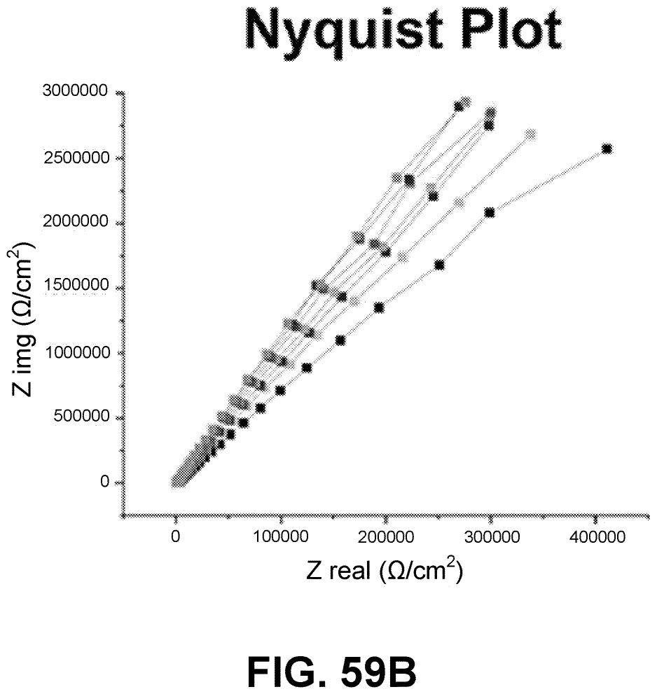

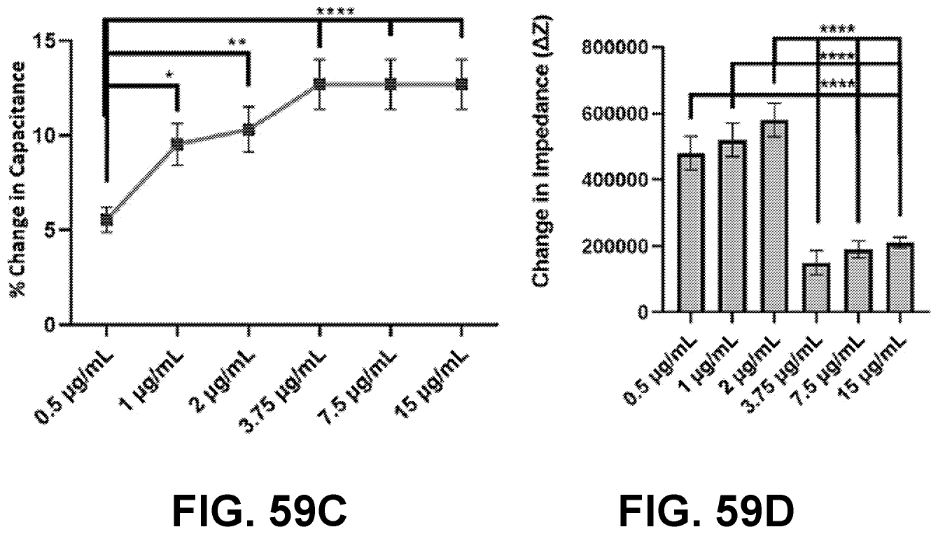

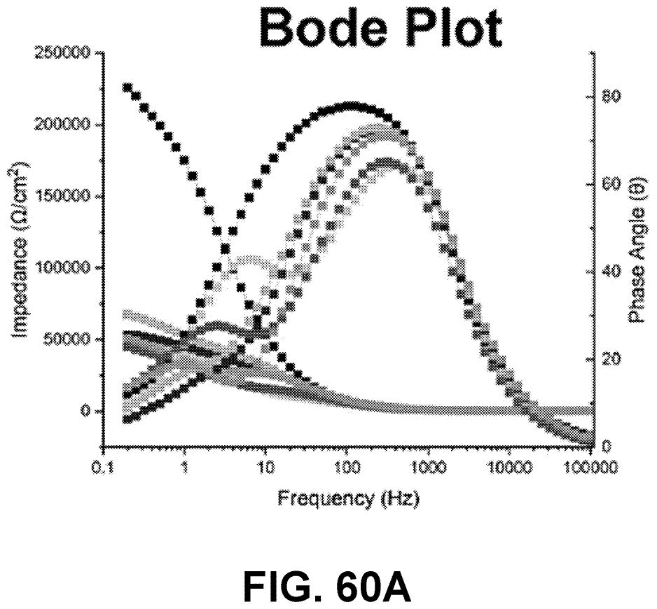

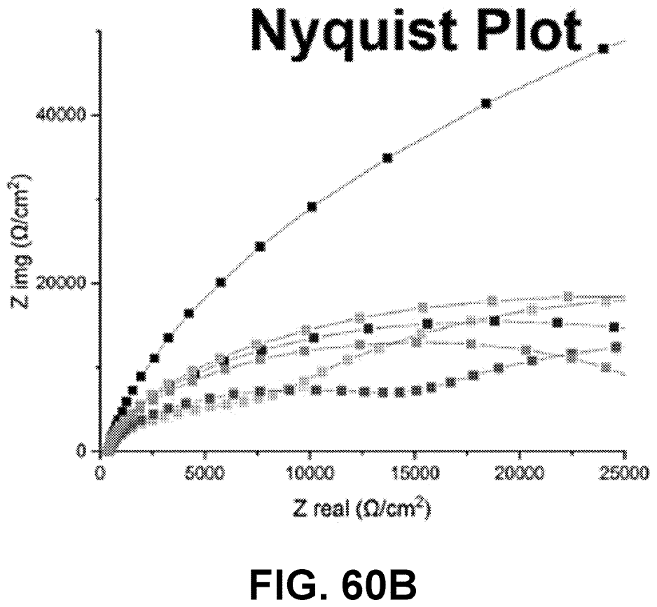

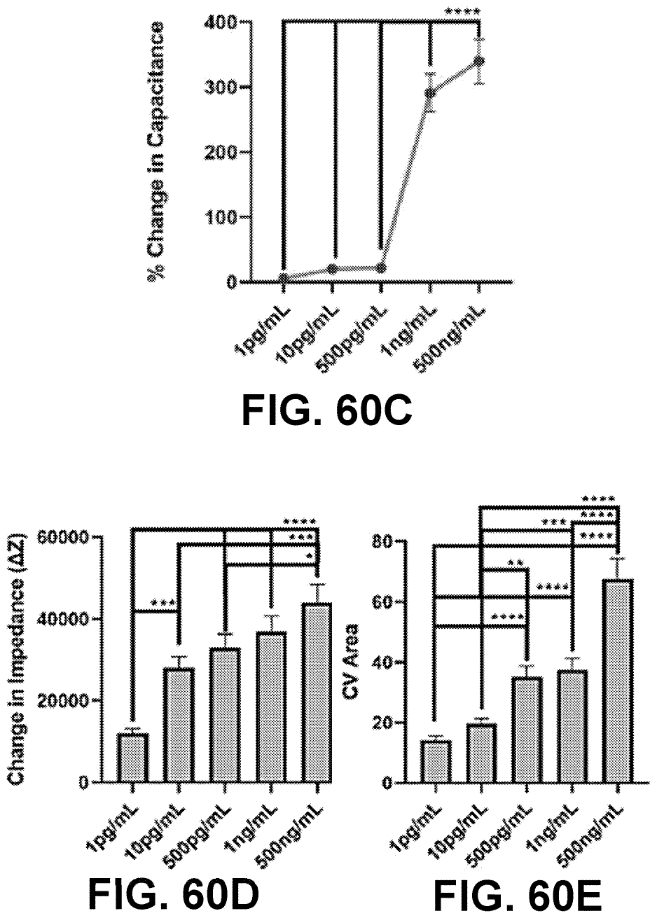

After conducting the antibody optimization and protein saturation experiments, the sensitivity of the different electrodes was evaluated by repeating the antibody saturation and protein calibration experiments with each electrode. The electrochemical results from the antibody calibration and protein saturation experiments are presented in FIGS. 59A-59D and 60A-60E respectively. Similar to the gold electrode, the graphene-nanoparticle-coated electrode had an optimal antibody concentration of 2 μg/mL as determined by a maximum change in impedance value (FIG. 59D). The approximately 2-fold increase in impedance values from the protein calibration experiments with the graphene-nanoparticle-coated electrode in comparison to the gold electrode suggests that the graphene-nanoparticle coating increases the sensitivity of the electrochemical biosensor to the protein CA19-9 (FIG. 60A). Additionally, the increase in sensitivity was further confirmed by a higher percentage change in capacitance values for each concentration of CA19-9 protein on the graphene-nanoparticle-coated electrode (FIG. 60C). Collectively, these higher values suggest that the graphene coating allows the biosensor to detect the concentration of CA19-9 more accurately and thereby conclusively in the solution.

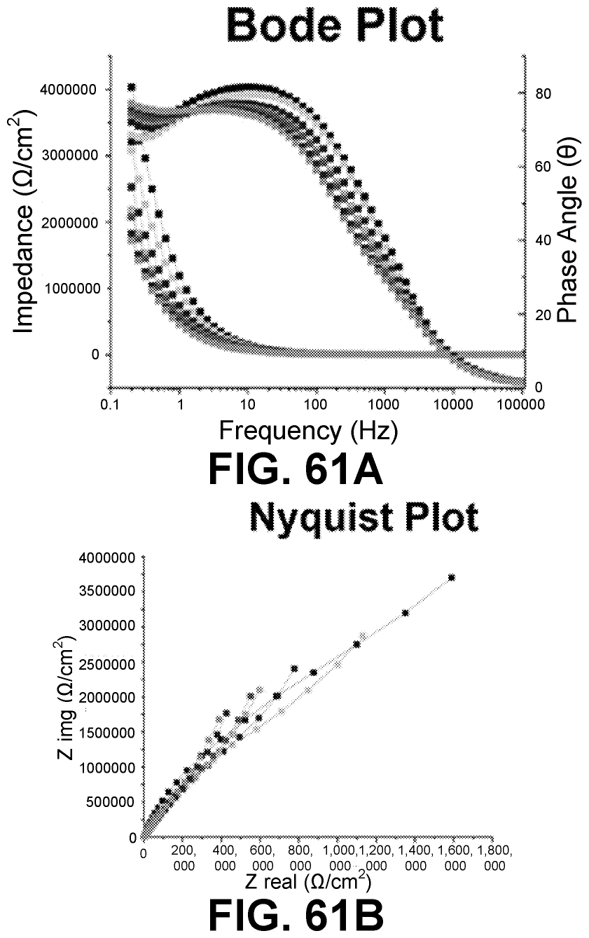

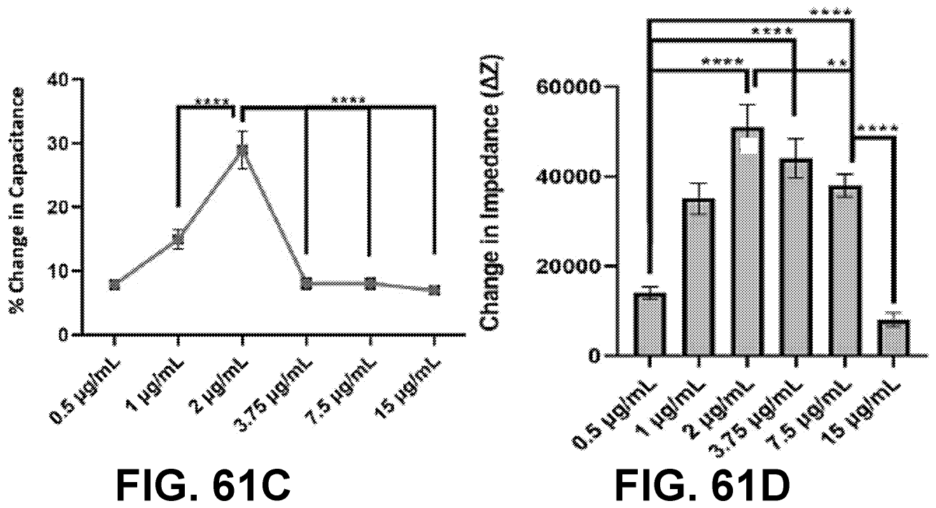

The antibody saturation and protein calibration experiments were repeated with a graphene electrode. Utilizing an optimal concentration of 2 μg/mL, CA19-9 antibody concentration was confirmed by the change in capacitance and impedance values of the antibody saturation experiments (FIGS. 61C and 61D). Additionally, the graphene oxide electrode provided the highest and the most distinct impedance values, allowing for the clear and accurate distinction between the different concentrations of CA 19-9 antibody (FIG. 61A). This distinction and accuracy were important to integrate a machine-learning model that provides targeted risk level analysis for patient samples with varying concentrations of CA19-9 antibody.

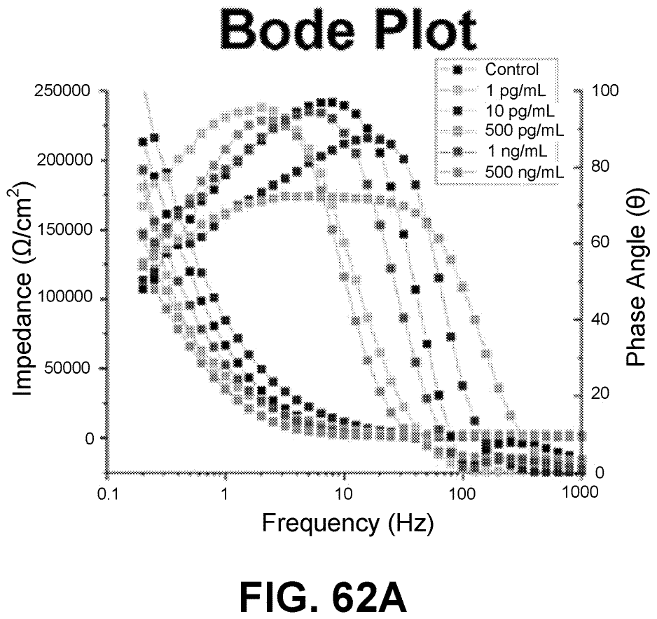

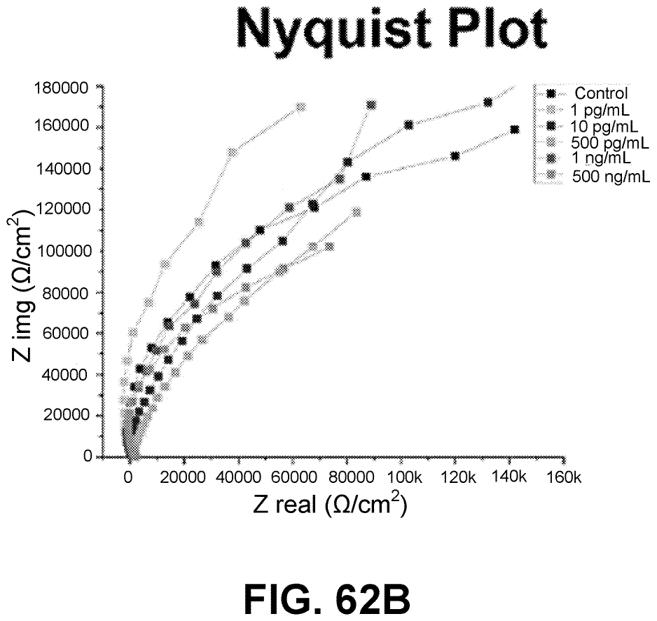

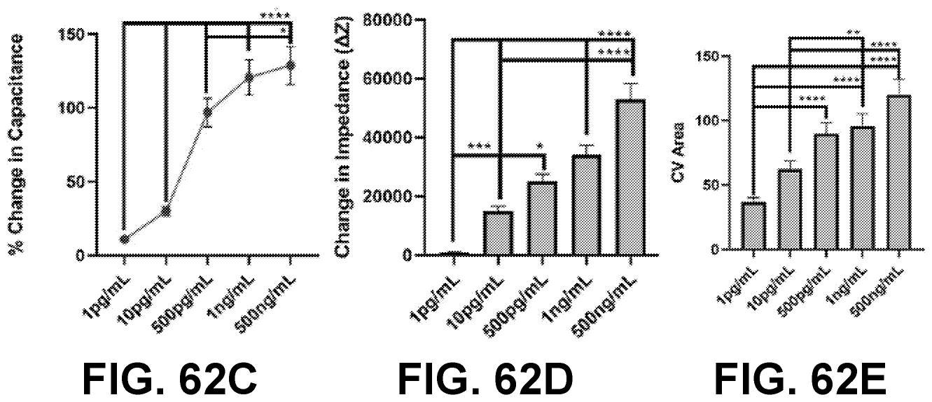

The CA19-9 protein calibration experiments demonstrated similar results as the presence of graphene caused a greater sensitivity to the varying protein concentrations. The pure graphene electrode depicted the most distinct impedance values for each concentration of CA19-9 protein compared to the gold and graphene-coated gold electrodes. These results suggest that the graphene electrode is more significant in increasing the sensitivity of the biosensor compared to the graphene-nanoparticle-coated electrode. Despite the graphene-coated gold electrode having the greatest change in capacitance values for each concentration of CA19-9 protein, the change in impedance values indicated that the graphene electrode had the greatest sensitivity, as it had the greater change in impedance values compared to the other two electrodes (FIG. 62D). Additionally, the graphene electrode protein saturation experiments produced higher CV Area values, further suggesting heightened sensitivity to CA19-9 protein (FIG. 62E). Despite a lack of consensus regarding a consistent indication of the highest sensitivity amongst the three electrodes by electrochemical values, both the graphene-nanoparticle-coated electrode and graphene electrode indicate the ability of graphene oxide to increase the sensitivity of the biosensor to the pancreatic cancer biomarker CA19-9 compared to a gold electrode with no graphene oxide.

In Vitro Biological Analysis: ELISA Test—to Compare the Sensitivity of the Developed Electrochemical Biosensor with the Conventional Biological Assay:

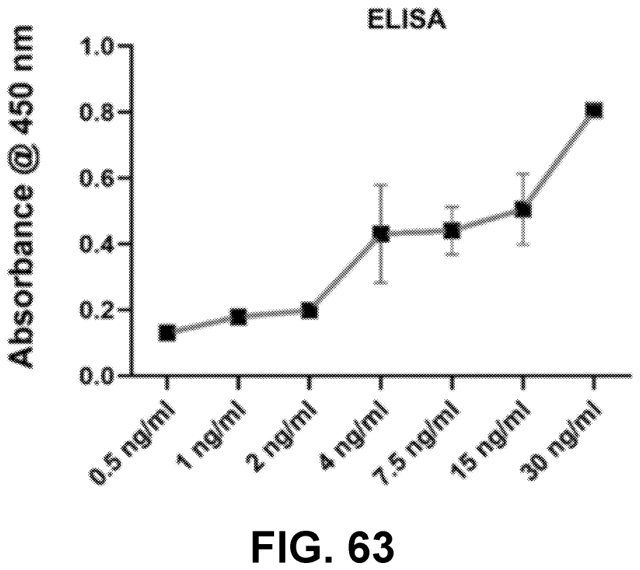

To biologically characterize and confirm the presence of the differing concentrations of CA19-9 protein on the surface of the biosensor, an antibody-linked ELISA assay was performed. Results from the ELISA test confirm the detection of the CA19-9 protein at varying concentrations (FIG. 63). However, there are low absorbance values, and thus sensitivity at 0.5 ng/ml, indicating that the sensor may not be capable of detecting protein concentrations below 0.5 ng/ml.

### Development of Automation Model and Interpretation of the Electrochemical Results Using a Machine Learning Model

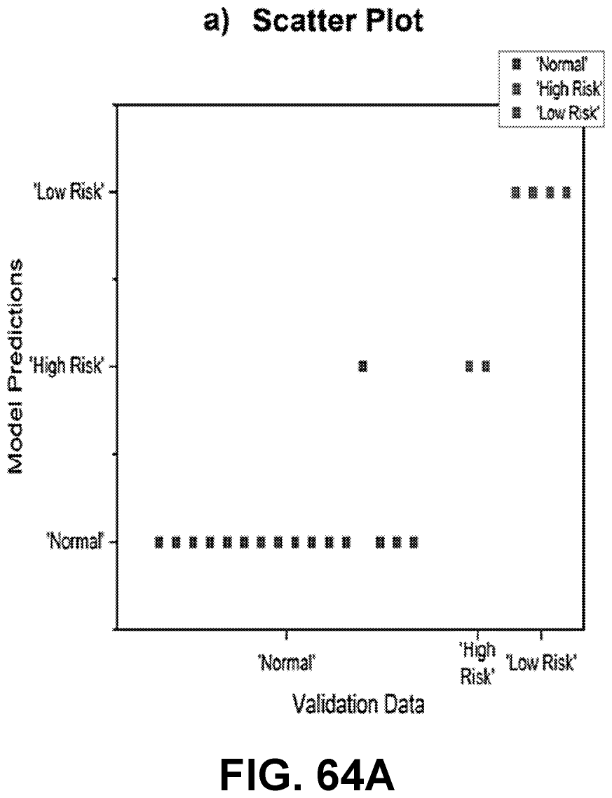

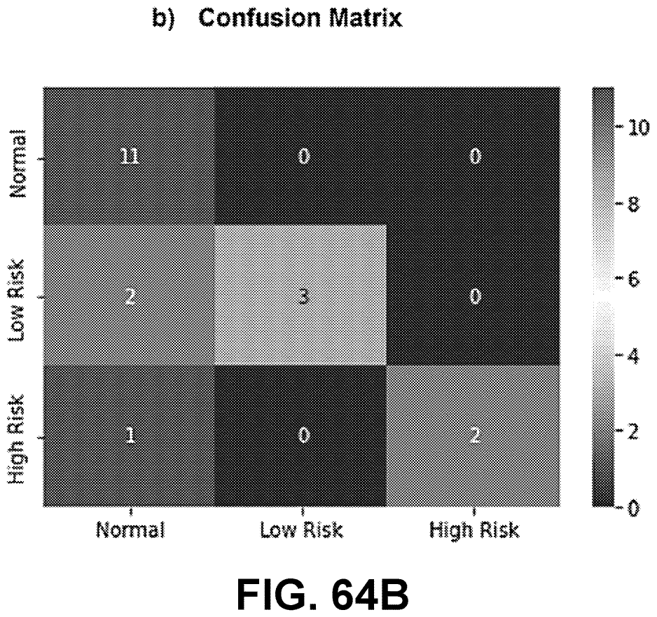

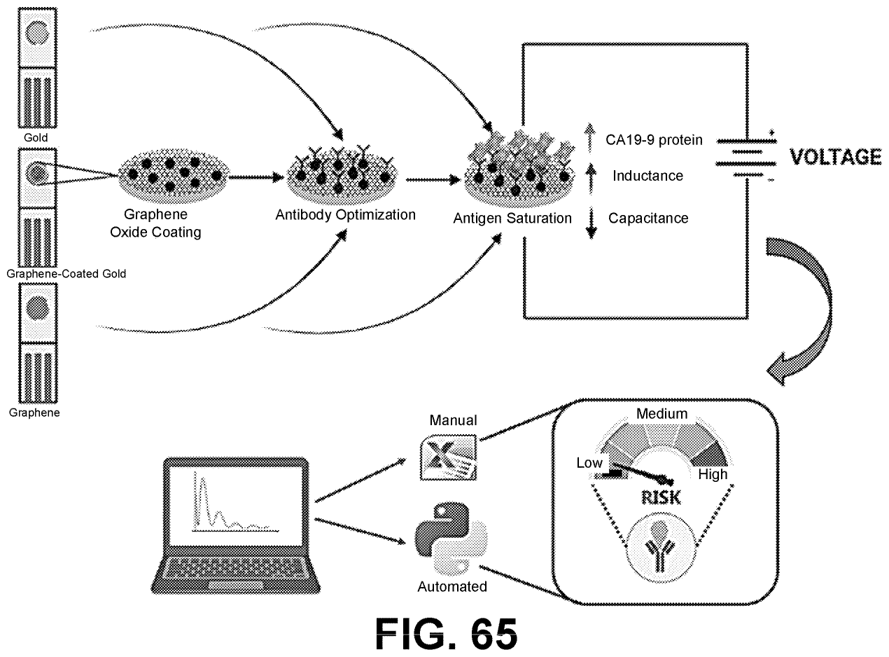

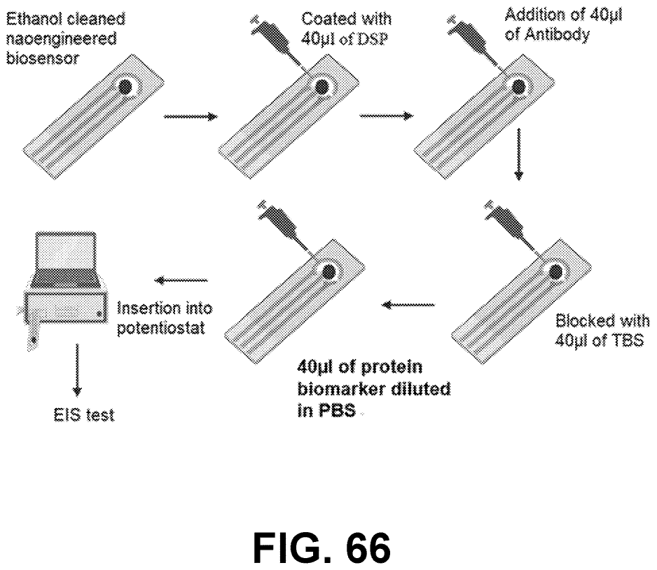

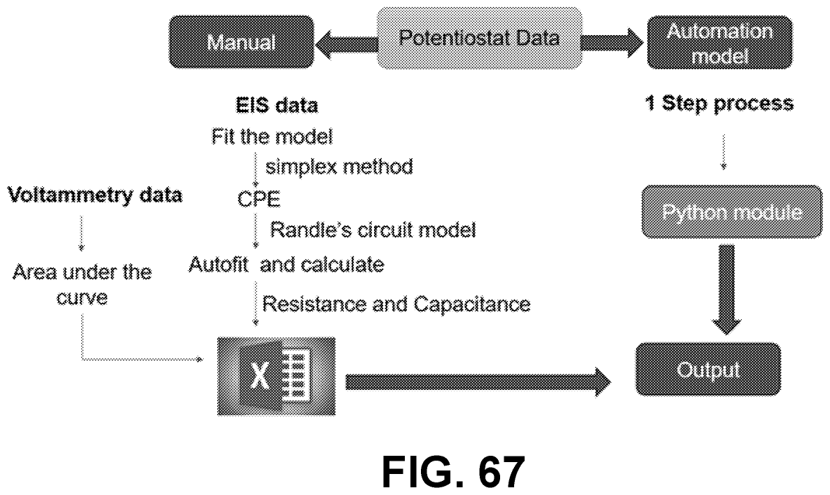

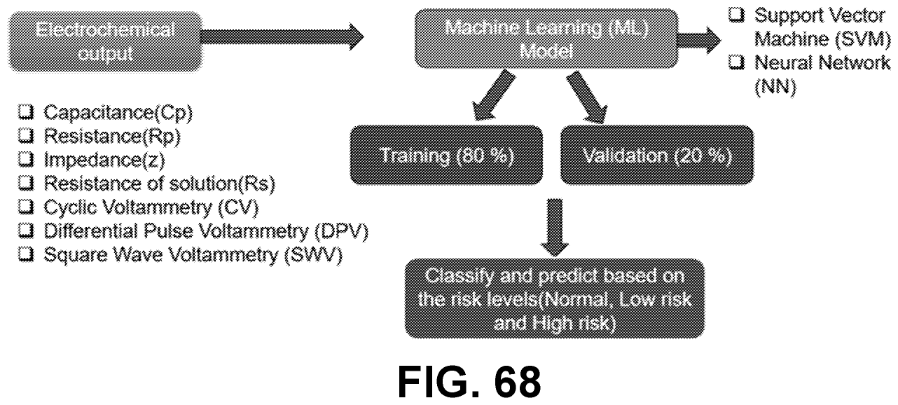

The confusion matrix obtained from machine learning procedures demonstrated 95% percent accuracy between predicted risk levels for varying samples of CA19-9 protein and true risk levels for the samples (FIGS. 64A and 64B). Thus, the high level of accuracy confirms the ability of biosensors and machine learning systems to match detected protein concentrations with the corresponding pancreatic cancer risk levels.

### Discussion

The results obtained from the electrochemical antibody saturation and protein calibration studies provide a thorough assessment of the efficacy of different electrodes in detecting varied concentrations of CA19-9 protein. The study utilized gold, graphene-nanoparticle-coated, and graphene electrodes, each exhibiting distinct sensitivity by detecting variations in impedance, capacitance, and other electrochemical characteristics. This comprehensive analysis will explore the importance of every experimental element and its consequences on the advancement of sophisticated biosensors used in the detection of pancreatic cancer biomarkers.

Gold Electrode as Control: The initial tests using the gold electrode served as a control to establish a baseline for comparison. These investigations determined that an appropriate antibody concentration of 2 μg/mL should be used for future experiments. The discernible differentiation among various CA19-9 protein concentrations in the protein saturation experiments confirmed the sensitivity of the electrochemical biosensor. These findings were not only used as a control for future studies but also emphasized the significance of having a uniform process for antibody optimization.

Electrode coated with graphene oxide nano colloids solution: The electrode coated with graphene oxide nanoparticles showed significant improvements in sensitivity. The recommended antibody concentration alignment with the gold electrode indicates a standardized methodology applicable to various electrode materials. The impedance values during protein calibration trials showed an approximately two-fold increase, indicating that the graphene-nanoparticle coating enhanced the biosensor's sensitivity to CA19-9 protein. The greater increase in capacitance values provided additional evidence that the graphene-nanoparticle coating enabled more precise and sophisticated detection of protein concentrations. The graphene oxide-coated gold electrode demonstrated remarkable sensitivity compared to other electrodes. The electrodes that exhibited the highest and most pronounced impedance values during antibody optimization and protein saturation trials indicated improved performance compared to the other electrodes. The graphene electrode's capacity to offer precise and unique differentiations among various concentrations of CA19-9 antibody is of utmost importance, particularly when considering its possible integration with machine learning models for risk level monitoring.

Impact of Graphene Oxide on Sensitivity: Although there were differences in electrochemical values among the three electrodes, it was generally agreed that both the graphene-nanoparticle-coated and graphene electrodes showed increased sensitivity to the CA19-9 protein compared to the gold electrode. The findings highlight the notable influence of graphene oxide in enhancing the sensitivity of the biosensor, indicating promising prospects for improving the ability to detect pancreatic cancer at an early stage.

Biological Characterization using ELISA: The verification of varying levels of CA 19-9 protein on the biosensor's surface by an ELISA experiment, which involved the use of an antibody, offered biological validation. The findings, meanwhile, suggested possible constraints in identifying protein quantities below 0.5 ng/ml, prompting inquiries over the biosensor's minimum detectable limit.

Integration of machine learning into the biosensor system yielded a remarkable 95% accuracy in predicting risk levels for different CA19-9 protein samples. The biosensor's high degree of accuracy not only confirmed its reliability but also demonstrated the promise of integrating sophisticated sensing technologies with artificial intelligence for accurate risk assessment in diagnosing pancreatic cancer.

The study's findings have significant ramifications for the advancement of biosensors in cancer diagnostics. The heightened sensitivity provided by graphene oxide, as demonstrated by the electrodes covered with graphene and graphene nanoparticles, indicates a possible approach to boost the early detection of pancreatic cancer.

In summary, this research provides a comprehensive analysis of the potential of graphene oxide in biosensor applications by combining electrochemical studies, biological characterization, and machine learning integration. The findings provide useful insights for the ongoing efforts to advance diagnostic technology, highlighting the significance of interdisciplinary approaches in the field of medical research and personalized medicine.

### Conclusion

The research investigated the sensitivity of several electrodes (gold, graphene-nanoparticle-coated, and graphene) in detecting CA19-9 protein concentrations, finding differential electrochemical responses.

Graphene electrodes displayed higher sensitivity when compared to both gold and graphene-nanoparticle-coated electrodes, demonstrating its potential for accurate detection of fluctuating CA19-9 antibody concentrations.

It was concluded that graphene oxide, in both nanoparticle-coated and pure forms, boosted biosensor sensitivity to the CA19-9 protein when compared to a gold electrode that did not include graphene oxide.

The presence of CA19-9 protein was verified using an ELISA test, albeit lower absorbance values showed possible problems in detecting quantities below 0.5 ng/ml.

Machine learning integration yielded a stunning 95% accuracy in predicting risk levels for diverse CA19-9 protein samples, highlighting the promise of biosensors and machine learning in cancer risk assessment.

### Clinical Significance

Early Pancreatic Cancer Detection: The biosensor's increased sensitivity, particularly with graphene oxide incorporation, has the potential to allow for early detection of the CA19-9 protein, allowing for prompt diagnosis of pancreatic cancer.

Customized Treatment Strategies: The accurate and sensitive detection of variable CA19-9 concentrations enables doctors to develop individualized treatment regimens for individuals, allowing for a more detailed assessment of disease progression.

Improved Patient Outcomes: By commencing therapies at earlier, more controllable stages of pancreatic cancer, early identification, and individualized therapy options lead to improved patient outcomes, increasing the chance of successful treatment.

Reduced Invasive Procedures: Reliable biosensors have the potential to eliminate the need for invasive diagnostic procedures, making monitoring pancreatic cancer biomarkers less difficult and more patient friendly.

Integration with Machine Learning for Risk Assessment: As proven in the research, the effective integration of biosensors with machine learning models offers a strong tool for accurate risk level assessment, assisting clinicians in making informed choices for patient care.

### Example 8—A Point of Care Electrochemical Biosensor for Early Detection & Monitoring of Periprosthetic Joint Infection

### Introduction

According to the 2024 American Joint Replacement Registry, there are over 1.2 million combined total hip and knee replacements performed annually. Despite implant efficacy, more than 18% of implants require revision. Periprosthetic joint infection (PJI) is the primary cause of implant revisions, affecting 21.4% of hip implant revisions and 33.1% of knee implant revisions. Existing testing methods, such as arthrocentesis and imaging, are reactionary and fail to predict joint infection when preventable. Novel electrochemical biosensors have emerged as highly sensitive, affordable, and convenient ways to diagnose and detect disease biomarkers. Interleukin-6 (IL6) and C-reactive protein (CRP) are strongly recommended by the American Association for Orthopedic Surgeons as predictors of PJI. Various embodiments of the present disclosure provide a point-of-care electrochemical biosensor that quantitatively detects IL-6 and CRP protein biomarkers to enable rapid and accurate early diagnosis of periprosthetic joint infection.

### Methods

For biosensor modeling, an optimal antibody saturation was determined using dilutions of 0, 0.25, 0.5, 1, and 1.25 μg/mL IL6+CRP antibody. For protein calibration, the risk levels for IL6 include normal (0, 2 pg/mL), low risk (5, 10 pg/mL), and high risk (20, 50 pg/mL). The risk levels for CRP include normal (<3 μg/mL), low risk (3-10 μg/mL), and high risk (10+μg/mL). Antibody-Antigen binding was performed by adding DSP, protein, TBS Superblock, Primary IL6 Antibody, and then Secondary FITC Antibody. Fluorescent imaging was then performed to visualize the protein, along with ELISA protein quantification, and BCA protein concentration. The treated supernatant was collected and EIS and CV were performed. An SVM machine learning model was trained to leverage EIS and CV data to classify serum as normal, low risk, and/or high risk.

### Results

For IL6, the optimal antibody saturation was found to be 1 μg/mL. For CRP, the optimal antibody saturation was found to be 0.5 μg/mL. For the protein calibration, there was a general increase in impedance change with increasing protein concentration in both IL-6 and CRP. Similar patterns were noted in capacitance and resistance graphs. The CV area decreased with concentrations.

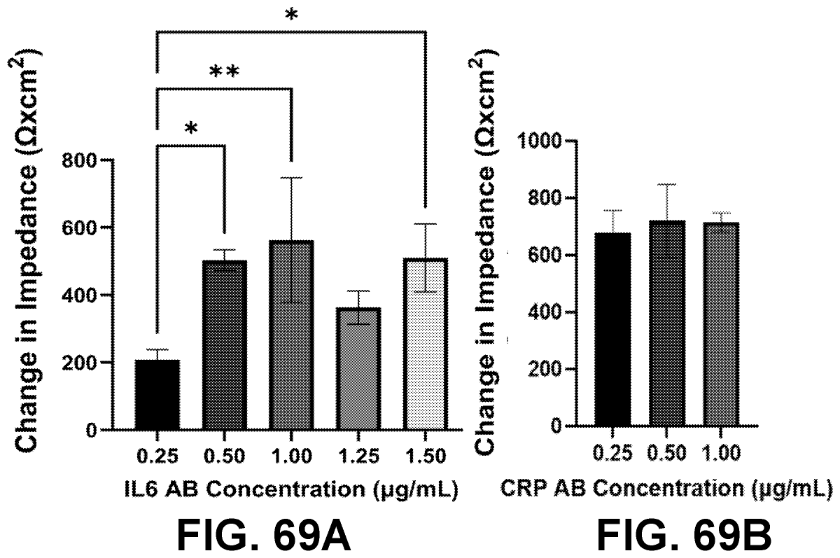

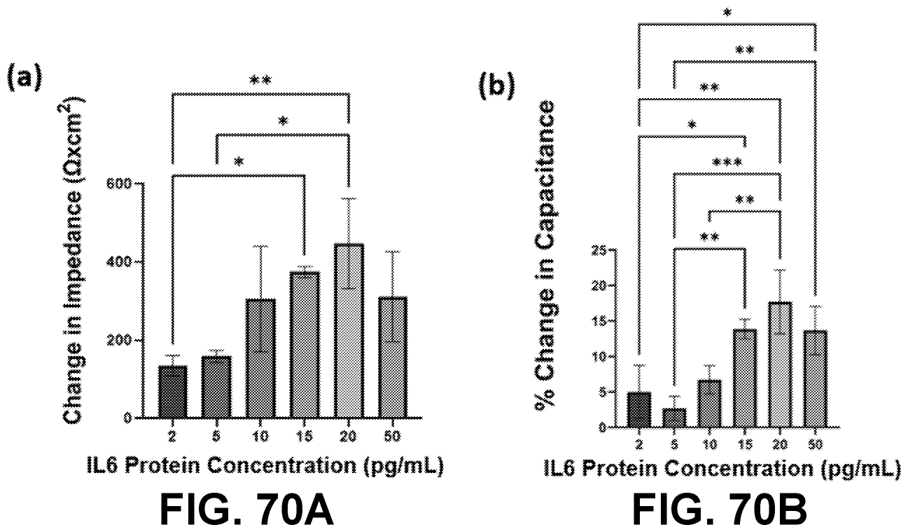

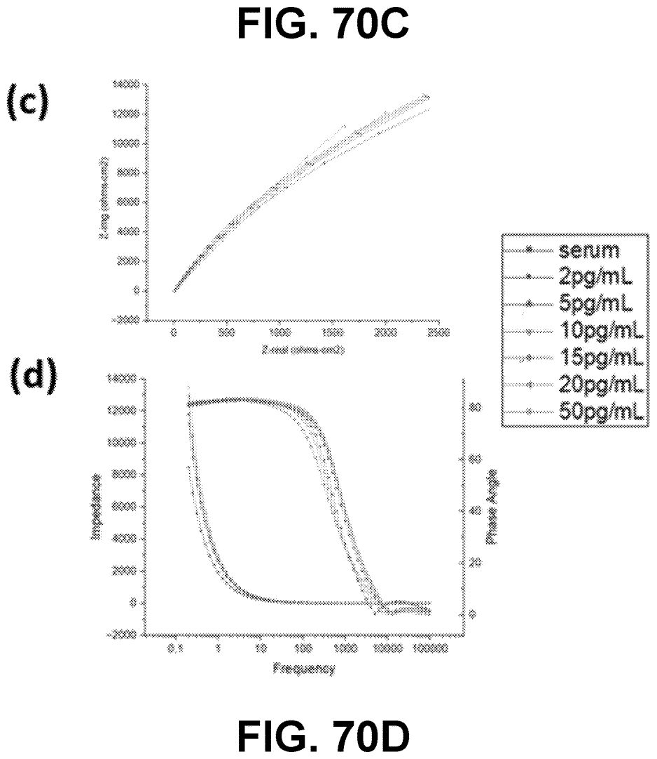

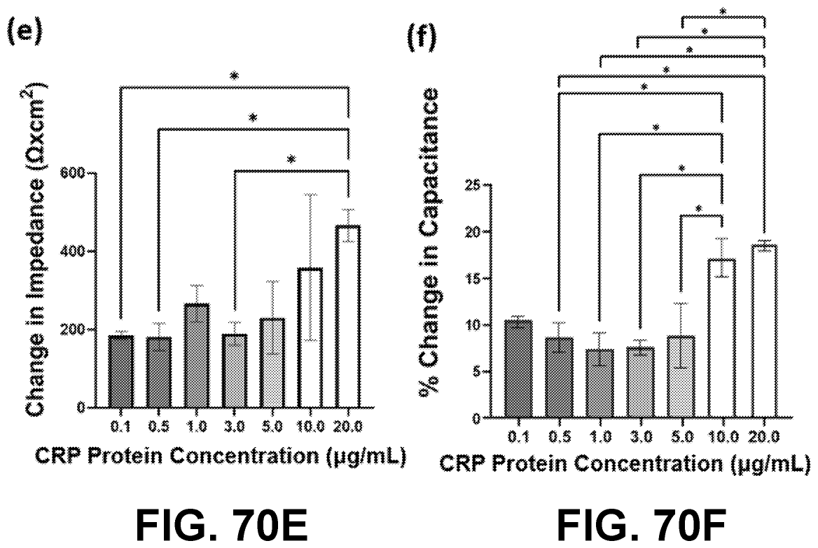

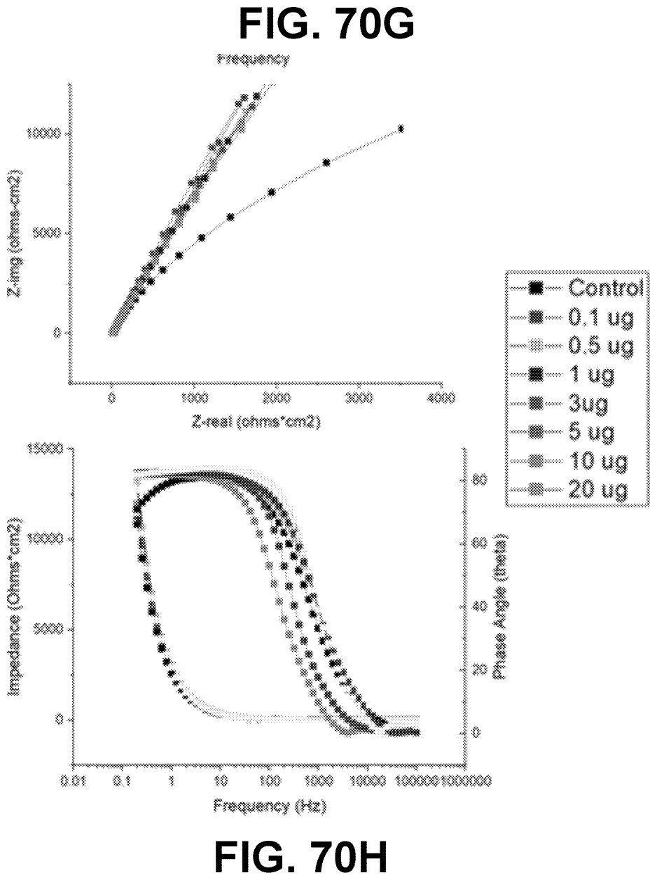

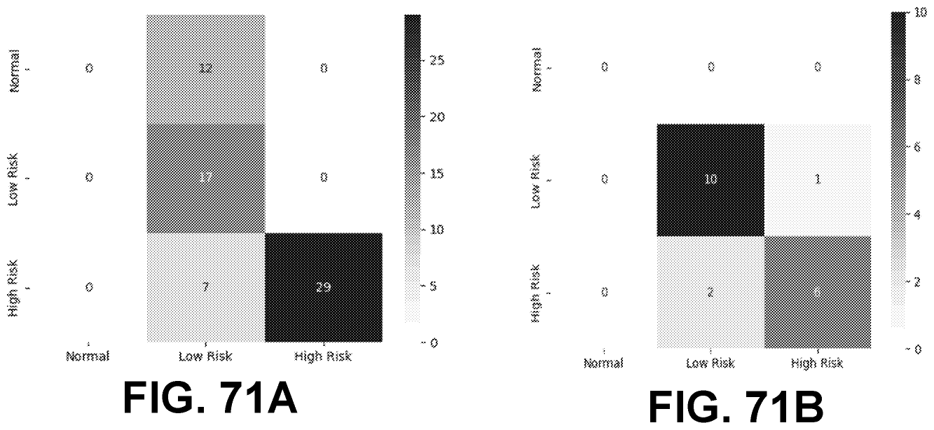

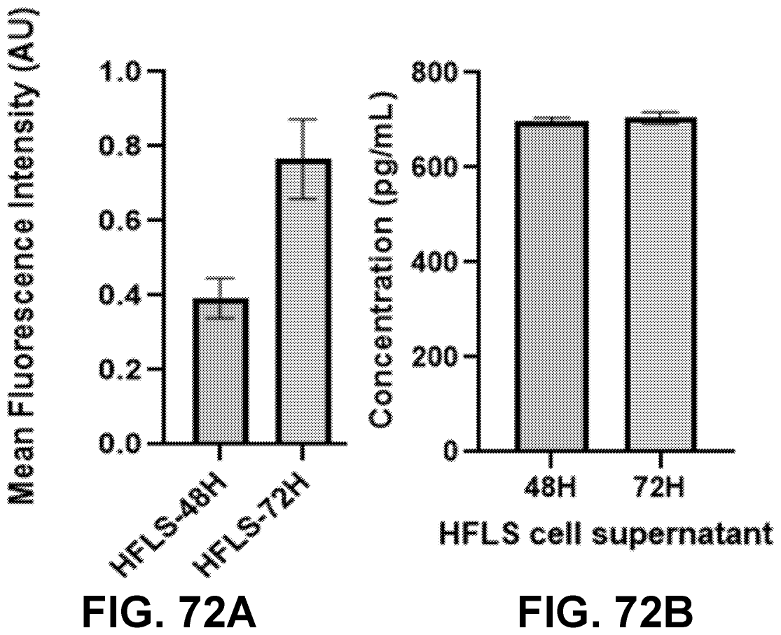

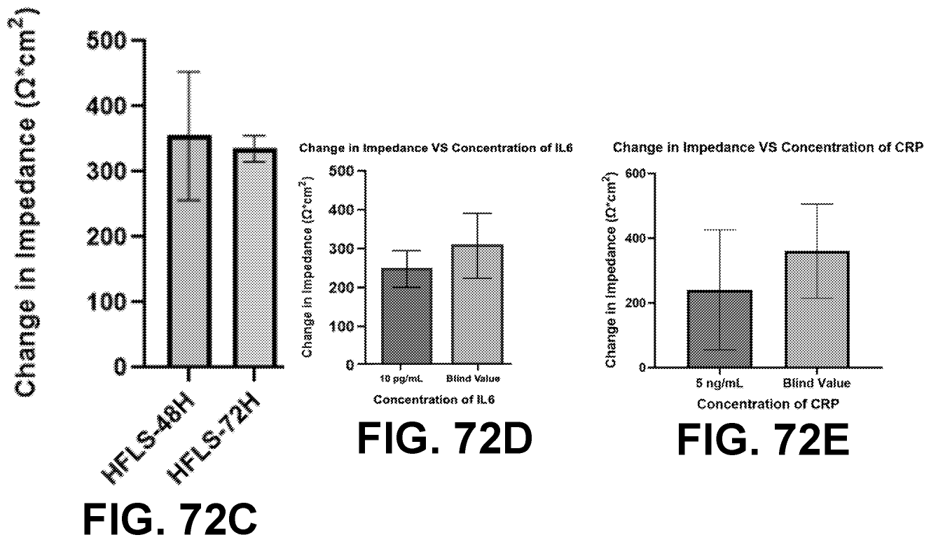

The trained SVM model yielded a 70.77% accuracy for IL6 and 72.41% accuracy for CRP. Additional results can be found in FIGS. 69A and 69B, FIGS. 70A-70H, FIGS. 71A and 71B, and FIGS. 72A-72E.

### Discussion

An at-home biosensor can be used to detect IL6/CRP and predict PJI. Early treatment and antibiotics can avoid revision surgery. To aid in early detection, an SVM model was successfully trained to predict a risk level associated with PJI based on detection and quantification of IL6 and CRP protein biomarkers.

### Example 9—Proof-of-Concept for Salivary COMP Detection for Early Osteoarthritis Using an AI-Driven Nano-Engineered Biosensor

### Introduction

Osteoarthritis (OA) is a leading cause of pain and disability worldwide and disproportionately affects rural and underserved populations with limited access to specialty care. Current diagnostics such as x-rays detect OA only after irreversible cartilage damage has occurred. Cartilage Oligomeric Matrix Protein (COMP) is a promising biomarker associated with cartilage turnover and OA progression. Saliva is an inexpensive, non-invasive diagnostic fluid but remains largely unexplored for OA biomarker detection.

### Methods

For biosensor development, a gold electrode was modified with graphene oxide to increase sensitivity. COMP antibodies were then immobilized at varying concentrations (0.25-1.5 μg/mL) by antibody saturation. Different COMP protein concentrations in serum and saliva were tested by protein calibration. Electrochemical impedance spectroscopy (EIS) and cyclic voltammetry (CV) data were used for signal measurement. The risk levels for COMP concentration in serum were categorized as normal (<7 μg/mL), low risk (7-15 μg/mL), and high risk (>20 μg/mL). The risk levels for COMP concentration in saliva were categorized as normal (<7 ng/ml), low risk (7-15 ng/ml), and high risk (>20 ng/ml).

For biological validation, human fibroblast-like synoviocytes (HFLS) were stimulated with LPS and LTA to mimic inflammatory OA environment. Confocal microscopy imaging was used to confirm antigen-antibody binding. COMP concentrations were validated using ELISA.

For the machine learning model, electrochemical parameters from EIS were used to train a Support Vector Machine (SVM) model. The model classified OA risk based on COMP concentration levels.

### Discussion

The optimal antibody saturation was found to be 1 μg/mL. A nano-engineered biosensor successfully detected COMP in saliva and serum. Confocal imaging showed increased fluorescence with the increase in concentration. LPS and LTA treated HFLS cells showed about 13.77 μg/mL and 13.45 μg/mL respectively. While the ELISA results showed 18.55 μg/mL and 15.25 μg/mL for the same. The SVM ML model showed an accuracy of 68.18% and 72.73% in serum and saliva concentrations of COMP.

### Results

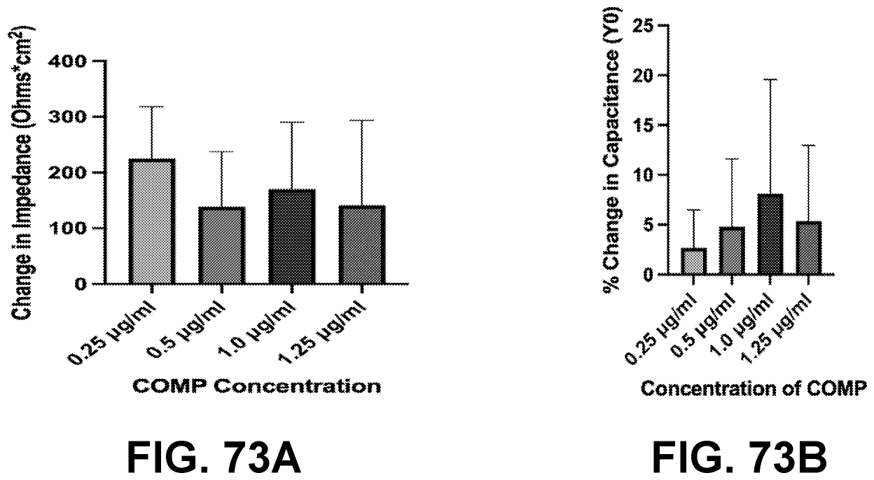

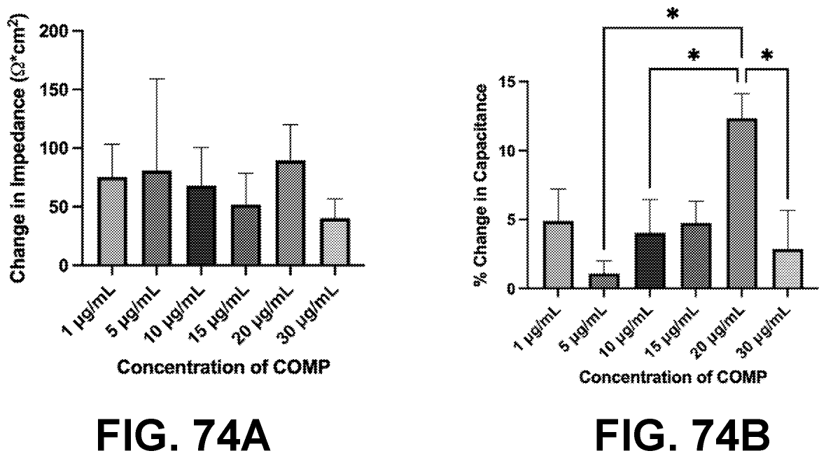

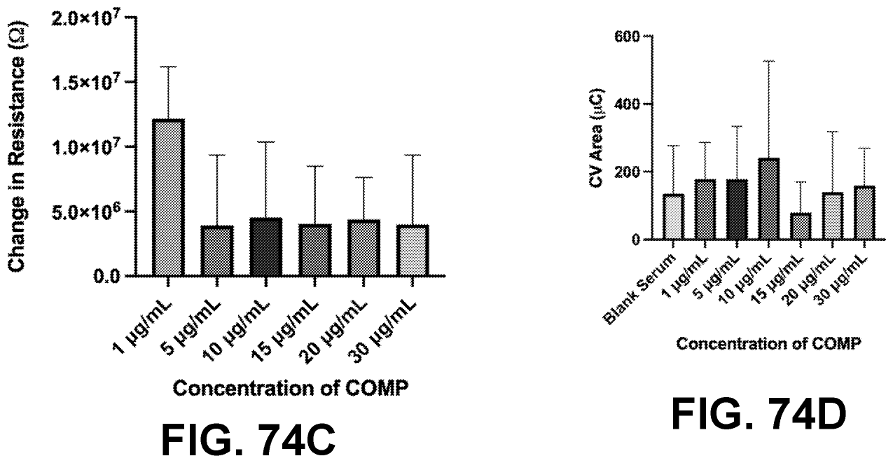

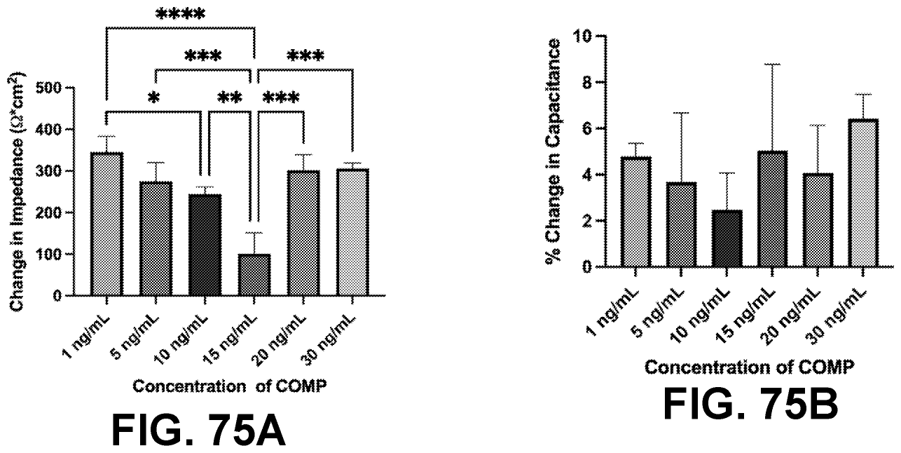

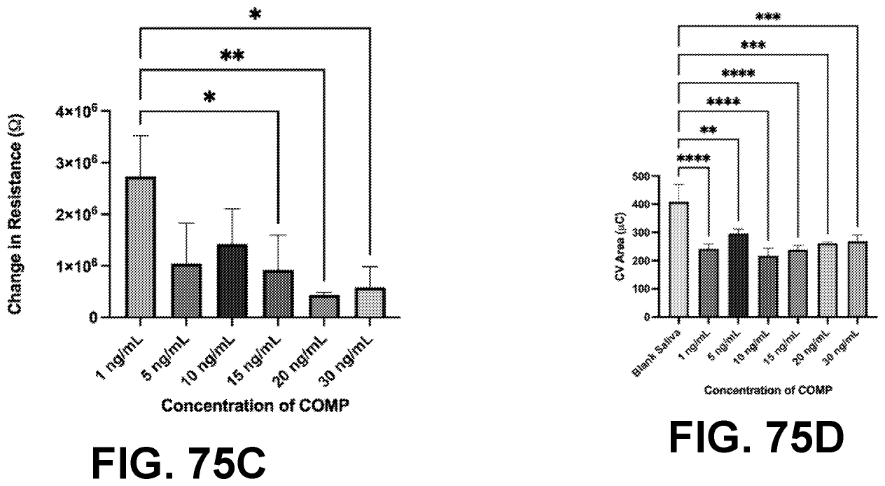

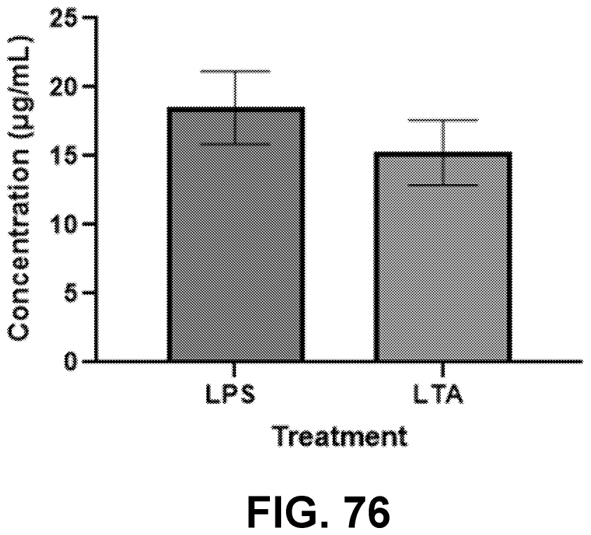

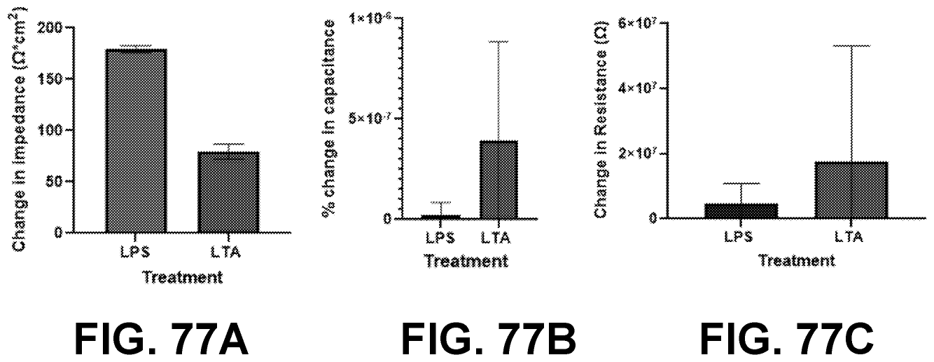

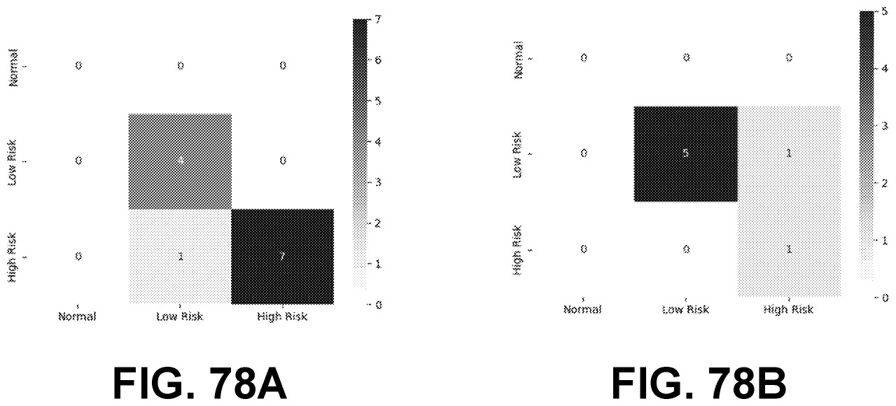

Results can be seen in FIGS. 73A and 73B, FIGS. 74A-74D, FIGS. 75A-75D, FIG. 76, FIGS. 77A-77C, and FIGS. 78A and 78B.

### Conclusion

The feasibility of detecting COMP in saliva using a nano-engineered electrochemical biosensor was demonstrated. Machine learning classification demonstrated accuracy for OA risk prediction. A non-invasive salivary diagnostic tool for early osteoarthritis detection was established. Saliva can be used as a non-invasive diagnostic fluid that can reflect systemic disease biomarkers. A portable point-of-care test could enable earlier OA diagnosis, particularly in rural and underserved populations. Integration of machine learning can further improve biomarker detection and diagnostic accuracy.

A phrase, such as “at least one of X, Y, or Z,” unless specifically stated otherwise, is to be understood with the context as used in general to present that an item, term, etc., can be either X, Y, or Z, or any combination thereof (e.g., X, Y, and/or Z). Similarly, “at least one of X, Y, and Z,” unless specifically stated otherwise, is to be understood to present that an item, term, etc., can be either X, Y, and Z, or any combination thereof (e.g., X, Y, and/or Z). Thus, as used herein, such phrases are not generally intended to, and should not, imply that certain embodiments require at least one of either X, Y, or Z to be present, but not, for example, one X and one Y. Further, such phrases should not imply that certain embodiments require each of at least one of X, at least one of Y, and at least one of Z to be present.

Although embodiments have been described herein in detail, the descriptions are by way of example. The features of the embodiments described herein are representative and, in alternative embodiments, certain features and elements may be added or omitted. Additionally, modifications to aspects of the embodiments described herein may be made by those skilled in the art without departing from the spirit and scope of the present disclosure described herein, the scope of which are to be accorded the broadest interpretation so as to encompass modifications and equivalent structures.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

Therefore, at least the following is claimed:

1. An electrochemical biosensor for detecting a biomarker in a biological fluid, comprising:
a working electrode, wherein the working electrode comprises a gold electrode coated with graphene oxide nanoparticles;
a capture agent conjugated to the graphene oxide nanoparticles, wherein the capture agent is configured to selectively bind to a target biomarker in the biological fluid;
a reference electrode; and
a counter electrode, wherein the electrochemical biosensor is configured to detect one or more changes in an electronic property of the working electrode resulting from binding of the target biomarker to the capture agent.

2. The electrochemical biosensor of claim 1, wherein the capture agent comprises an antibody or an antibody fragment.

3. The electrochemical biosensor of claim 2, wherein the antibody is selected from the group consisting of anti-MMP-9, anti-CA19-9, anti-CEA, anti-CA242, anti-adiponectin, anti-ECM1, anti-ALP, anti-BCA-225, anti-Haptoglobin, IL6 antibody, CYFRA21-1 antibody, S100A7 antibody, CA-125 antibody, anti-BNP, anti-C Reactive Protein, anti-cardiac troponin T, anti-amyloid-β 1-42, p-TAU antibody, anti-Lactoferrin, acetylcholinesterase antibody, anti-cartilage oligomeric matrix protein, anti-prosurfactant protein B, anti-serpinB3, squamous cell carcinoma antigen antibody, MMP-12 antibody, anti-NT-proBNP, anti-myeloperoxidase, anti-Ox-LDL, anti-lactoferrin, SURVIVIN antibody, BDNF monoclonal antibody, IL-1β antibody, anti-osteocalcin, anti-CTX, anti-TNF-α, anti-MMP-8, and anti-GSH.

4. The electrochemical biosensor of claim 1, wherein the biological fluid is selected from the group consisting of saliva, urine, serum, and blood.

5. The electrochemical biosensor of claim 1, wherein the target biomarker is associated with a disease selected from the group consisting of cancer, stroke, periodontitis, diabetes, cardiovascular disease, Alzheimer's Disease, osteoarthritis, cardiovascular atrial fibrillation, atherosclerosis, mental disorders, periprosthetic joint infection, autism, osteoporosis, peri implantitis, and obesity.

6. The electrochemical biosensor of claim 1, further comprising an organic linker molecule conjugated to the graphene oxide nanoparticles, wherein the organic linker molecule is conjugated to the capture agent.

7. The electrochemical biosensor of claim 6, wherein the organic linker molecule comprises dithiobis succinimidyl propionate.

8. A method of detecting a biomarker associated with a disease in a biological fluid sample, comprising:
contacting the biological fluid sample with a working electrode of an electrochemical biosensor, wherein the working electrode comprises a surface coated with graphene oxide nanoparticles and a capture agent conjugated to the graphene oxide nanoparticles;
detecting, using the electrochemical biosensor, one or more changes in an electronic property of the working electrode resulting from binding of the biomarker to the capture agent;
determining, using a computing device, electrochemical data based at least in part on the one or more changes in the electronic property of the working electrode detected by the electrochemical biosensor;
inputting, by the computing device, the electrochemical data into a machine-learning (ML) model trained to analyze the electrochemical data to determine a risk level associated with the disease; and
determining, by the computing device, the risk level associated with the disease based at least in part on the ML model.

9. The method of claim 8, wherein the capture agent comprises an antibody selected from the group consisting of anti-MMP-9, anti-CA19-9, anti-CEA, anti-CA242, anti-adiponectin, anti-ECM1, anti-ALP, anti-BCA-225, anti-Haptoglobin, IL6 antibody, CYFRA21-1 antibody, S100A7 antibody, CA-125 antibody, anti-BNP, anti-C Reactive Protein, anti-cardiac troponin T, anti-amyloid-β 1-42, p-TAU antibody, anti-Lactoferrin, acetylcholinesterase antibody, anti-cartilage oligomeric matrix protein, anti-prosurfactant protein B, anti-serpinB3, squamous cell carcinoma antigen antibody, MMP-12 antibody, anti-NT-proBNP, anti-myeloperoxidase, anti-Ox-LDL, anti-lactoferrin, SURVIVIN antibody, BDNF monoclonal antibody, IL-1β antibody, anti-osteocalcin, anti-CTX, anti-TNF-α, anti-MMP-8, and anti-GSH.

10. The method of claim 9, wherein the biological fluid sample is selected from the group consisting of saliva, urine, serum, and blood.

11. The method of claim 9, wherein the disease is selected from the group consisting of cancer, stroke, periodontitis, diabetes, cardiovascular disease, Alzheimer's Disease, osteoarthritis, cardiovascular atrial fibrillation, atherosclerosis, mental disorders, periprosthetic joint infection, autism, osteoporosis, peri implantitis, and obesity.

12. The method of claim 8, wherein the machine learning model is trained using one or more electrochemical parameters comprising impedance, capacitance, resistance, cyclic voltammetry area, or a combination of any thereof.

13. The method of claim 8, wherein the electrochemical data comprises a change in impedance, a change in capacitance, a change in resistance, or a combination of any thereof.

14. A disease risk prediction system, comprising:
an electrochemical biosensor comprising at least a working electrode, wherein a capture agent is disposed on a surface of the working electrode and is configured to selectively bind to a target biomarker associated with a disease, and wherein the electrochemical biosensor is configured to detect one or more changes in an electronic property of the working electrode based at least in part on the target biomarker binding to the capture agent;
a computing device comprising a processor and a memory; and
machine-readable instructions that, when executed by the processor, cause the computing device to at least:
determine electrochemical data based at least in part on the one or more changes in the electronic property of the working electrode detected by the electrochemical biosensor;
input the electrochemical data into a machine-learning (ML) model trained to analyze the electrochemical data to determine a risk level associated with the disease; and
determine the risk level associated with the disease based at least in part on the ML model.

15. The disease risk prediction system of claim 14, wherein the machine learning model comprises a support vector machine.

16. The disease risk prediction system of claim 14, wherein the risk level is normal, low risk, or high risk.

17. The disease risk prediction system of claim 14, wherein the machine learning model is trained using one or more electrochemical parameters comprising impedance, capacitance, resistance, cyclic voltammetry area, or a combination of any thereof.

18. The disease risk prediction system of claim 14, wherein the electrochemical data comprises a change in impedance, a change in capacitance, a change in resistance, or a combination of any thereof.

19. The disease risk prediction system of claim 14, wherein the capture agent comprises an antibody selected from the group consisting of anti-MMP-9, anti-CA19-9, anti-CEA, anti-CA242, anti-adiponectin, anti-ECM1, anti-ALP, anti-BCA-225, anti-Haptoglobin, IL6 antibody, CYFRA21-1 antibody, S100A7 antibody, CA-125 antibody, anti-BNP, anti-C Reactive Protein, anti-cardiac troponin T, anti-amyloid-β 1-42, p-TAU antibody, anti-Lactoferrin, acetylcholinesterase antibody, anti-cartilage oligomeric matrix protein, anti-prosurfactant protein B, anti-serpinB3, squamous cell carcinoma antigen antibody, MMP-12 antibody, anti-NT-proBNP, anti-myeloperoxidase, anti-Ox-LDL, anti-lactoferrin, SURVIVIN antibody, BDNF monoclonal antibody, IL-1β antibody, anti-osteocalcin, anti-CTX, anti-TNF-α, anti-MMP-8, and anti-GSH.

20. The disease risk prediction system of claim 14, wherein the disease is selected from the group consisting of cancer, stroke, periodontitis, diabetes, cardiovascular disease, Alzheimer's Disease, osteoarthritis, cardiovascular atrial fibrillation, atherosclerosis, mental disorders, periprosthetic joint infection, autism, osteoporosis, peri implantitis, and obesity.
